(19) United States
(12) Patent Application Publication
(10) Pub. No.: US 2026/0231945 A1
(43) Pub. Date: Aug. 13, 2026

(54) NEMATICIDAL COMPOUNDS, THEIR COMPOSITION AND USE THEREOF

(71) Applicants: Komal BHUKHANWALA, Mumbai (IN); Venkata Siva Prasada Rao LINGAM, Navi Mumbai (IN)

(72) Inventors: Komal BHUKHANWALA, Mumbai (IN); Venkata Siva Prasada Rao LINGAM, Navi Mumbai (IN)

(21) Appl. No.: 19/154,606

(22) PCT Filed: Feb. 19, 2024
(86) PCT No.: PCT/IN2024/050178

§ 371 Date: Aug. 7, 2025

(30) Foreign Application Priority Data
Feb. 17, 2023 (IN) 202321011330

(51) Int. Cl. A01N 47/44; A01P 5/00; C07C 279/14; C07D 209/08; C07D 209/44; C07D 213/53; C07D 233/64; C07D 239/26; C07D 263/32; C07D 263/56; C07D 277/64; C07D 307/52; C07D 333/22

(52) U.S. Cl. CPC A01N 47/44; A01P 5/00; C07C 279/14; C07D 209/08; C07D 209/44; C07D 213/53; C07D 233/64; C07D 239/26; C07D 263/32; C07D 263/56; C07D 277/64; C07D 307/52; C07D 333/22

(57) ABSTRACT

The present invention provides a compound of formula (I)

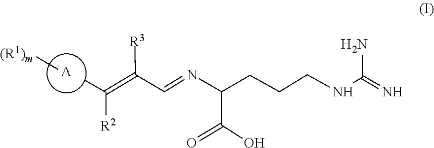

or salt thereof or N-oxide thereof, and their agriculture composition and their use for treating or controlling the parasitic nematode or the nematode infection in agriculture crops.

### FIELD OF THE INVENTION

The invention relates to compounds having nematocidal activity, their agricultural composition and use of such compounds for treating or controlling the parasitic nematode or the nematode infection in agriculture crops.

### BACKGROUND OF THE INVENTION

Nematodes are active, flexible, elongate, organisms that live on moist surfaces or in liquid environments, including films of water within soil and moist tissues within other organisms. Many species of nematodes have evolved to be very successful parasites of plants and animals and are responsible for significant economic losses in agriculture. Nematodes are known to affect the yield, growth, and health of crops and plants. The physiological changes in the host plant's roots caused by larvae and/or adult nematodes can lead to the formation of galls, which causes a disruption of the vascular system of the plant's roots. Root elongation can stop completely and inadequate supply of water and nutrients provided by the reduced root system can result, causing foliage chlorosis and/or wilt, as well as stunting of growth, any of which can result in low yield or death. In addition, nematodes can cause physiological effects leading to an increase in the susceptibility of plant roots to bacteria and/or fungi attack, including bacteria and/or fungi the plant would otherwise resist. Such attack can lead to extensive secondary decay and rotting. Hence, the control of plant parasitic nematodes is extremely important in achieving high crop efficiency.

Due to widespread development of resistance to anthelmintic agents in nematode parasites, nematodes continue to cause problems despite the available therapeutic agents. The need continues for new compounds which are more effective, less costly, less toxic, environmentally safer or have different modes of action.

### SUMMARY OF THE INVENTION

The present invention provides a compound of formula (I),

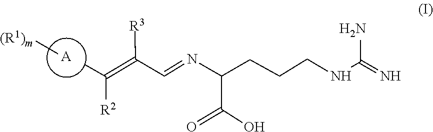

or agrochemically acceptable salt, stereoisomer, enantiomer, tautomer, or N-oxide thereof,
wherein, ring A, R¹, R², R³ and ‘m’ are as defined in detailed description. The present invention further relates to and the agriculture composition of compound of Formula (I) and their use for treating or controlling the parasitic nematode or the nematode infection in agriculture crops.

### DESCRIPTION OF THE INVENTION

### Definitions

In describing the embodiment of the invention, specific terminology is chosen for the sake of clarity. However, it is not intended that the invention be limited to the specific terms so selected and it is to be understood that such specific terms include all technical equivalents that operate in a similar manner to accomplish a similar purpose. It is understood that any numerical range recited herein is intended to include all subranges subsumed. Also, unless denoted otherwise percentage of components in a composition are presented as weight percent.

The terms “a” or “an”, as used herein, are defined as one or more than one. The terms “including” and/or “having”, as used herein, are defined as comprising (i.e., open language).

The terms “halogen” or “halo” means fluorine (fluoro), chlorine (chloro), bromine (bromo), or iodine (iodo).

The term “alkyl” refers to a straight or branched hydrocarbon chain radical that includes solely carbon and hydrogen atoms in the backbone, containing no unsaturation, having from one to six carbon atoms (i.e. C₁₋₆alkyl), and which is attached to the rest of the molecule by a single bond.

The term “C₁₋₆ alkyl” is an alkyl group that has from 1 to 6 carbon atoms. Non-limiting examples of alkyl groups include methyl, ethyl, n-propyl, 1-methylethyl (isopropyl), n-butyl, 2-methylpropyl (isobutyl), n-pentyl, 1,1-dimethylethyl (t-butyl), and 2,2-dimethylpropyl.

The term “C₂₋₆alkenyl” refers to a straight or branched hydrocarbon chain radical that includes solely carbon and hydrogen atoms in the backbone, containing at least one carbon-carbon double bond having from one to six carbon atoms, and which is attached to the rest of the molecule by a single bond.

The term “alkoxy” denotes an alkyl group attached via an oxygen linkage to the rest of the molecule (i.e. C₁₋₆alkoxy). Representative examples of such groups are —OCH₃ and —OC₂H₅.

The term “haloalkyl” refers to at least one halo group (selected from F, Cl, Br or I), linked to an alkyl group as defined above (i.e. haloC₁₋₆alkyl). Examples of such haloalkyl moiety include, but are not limited to, trifluoromethyl, trifluoroethyl, difluoromethyl and fluoromethyl groups.

The term “haloalkoxy” refers to an alkoxy group substituted with one or more halogen atoms (i.e. haloC₁₋₆alkoxy). Examples of “haloalkoxy” include but are not limited to fluoromethoxy, difluoromethoxy, trifluoromethoxy, 2,2,2-trifluoroethoxy, pentafluoroethoxy, pentachloroethoxy, chloromethoxy, dichlorormethoxy, trichloromethoxy and 1-bromoethoxy.

The term “hydroxyalkyl” refers to an alkyl group as defined above wherein one to three hydrogen atoms on different carbon atoms is/are replaced by hydroxyl groups (i.e. hydroxyC₁₋₆alkyl). Examples of hydroxyalkyl moiety include, but are not limited to —CH₂OH, —C₂H₄OH and —CH(OH)C₂H₄OH.

The term “aryl” refers to an aromatic radical having 6 to 14 carbon atoms (i.e. C₆₋₁₄aryl), including monocyclic, bicyclic and tricyclic aromatic systems, such as phenyl, naphthyl, tetrahydronaphthyl, indanyl, and biphenyl.

The term “heteroaryl” unless otherwise specified refers to substituted or unsubstituted 5- to 14-membered aromatic heterocyclic ring radical with one or more heteroatom(s) independently selected from N, O or S. The heteroaryl may be a mono-, bi- or tricyclic ring system. The heteroaryl ring radical may be attached to the main structure at any heteroatom or carbon atom that results in the creation of a stable structure. Examples of such heteroaryl ring radicals include, but are not limited to oxazolyl, isoxazolyl, imidazolyl, furyl, indolyl, isoindolyl, pyrrolyl, pyrazolyl, triazolyl, triazinyl, tetrazoyl, thienyl, thiazolyl, isothiazolyl, pyridyl, pyridinyl, pyrimidinyl, pyrazinyl, pyridazinyl, benzofuranyl, benzothiazolyl, benzoxazolyl, benzimidazolyl, benzothienyl, benzopyranyl, carbazolyl, quinolinyl, isoquinolinyl, quinazolinyl, cinnolinyl, naphthyridinyl, pteridinyl, purinyl, quinoxalinyl, quinolyl, isoquinolyl, thiadiazolyl, indazolyl, indolizinyl, acridinyl, phenazinyl and phthalazinyl.

The term “salt” includes salts prepared from bases or acids including inorganic or organic bases and inorganic or organic acids. Examples of such salts include, but are not limited to, acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide and valerate. Examples of salts derived from inorganic bases include, but are not limited to, aluminum, ammonium, calcium, copper, ferric, ferrous, lithium, magnesium, manganic, mangamous, potassium, sodium, and zinc.

As used herein a wavy line “ [symbol] ” indicates the point of attachment of the bond to the remainder of a molecule.

Certain compounds of present patent application are capable of existing in stereoisomeric forms (e.g. diastereomers and enantiomers) and geometrical forms (E and Z configuration). With respect to the overall compounds described by the general formula (I), the present patent application extends to these stereoisomeric forms, geometrical forms and mixtures thereof. To the extent prior art teaches synthesis or separation of particular stereoisomers, the different stereoisomeric forms of the present patent application may be separated from one another by the method known in the art, or a given isomer may be obtained by stereo specific or asymmetric synthesis. Tautomeric forms and mixtures of compounds described herein are also contemplated. It is also to be understood that compounds of the invention may exist in solvated forms (such as hydrates) as well as un-solvated forms, and that the invention encompasses all such forms.

The present invention provides a compound of formula (I),

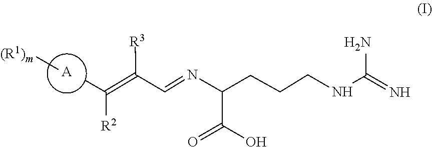

or agrochemically acceptable salt, stereoisomer, enantiomer, tautomer, or N-oxide thereof,
wherein,
oxazolyl, isoxazolyl, oxadiazolyl, pyrrolyl, pyrazolyl, imidazolyl, pyrimidinyl, indolyl, isoindolyl, indazolyl, benzofuryl, benzoxazolyl, and benzothiozolyl;
at each occurrence, R¹ is selected from hydrogen, halogen, —OH, —CN, C₁₋₆alkyl, C₁₋₆alkoxy, haloC₁₋₆alkyl, haloC₁₋₆alkoxy, C₂₋₆alkenyl, —CO(O)—C₁₋₆alkyl, —OC(O)—C₁₋₆alkyl and —OC(O)—C₆₋₁₄aryl;
R² and R³ is independently hydrogen, or C₁₋₆alkyl; and
‘m’ is an integer ranging from 0 to 4, both inclusive.

According to yet another embodiment, specifically provided are compounds of formula (I), in which R¹ is hydrogen, halogen, —OH, —CN, C₁₋₆alkyl, C₁₋₆alkoxy, haloC₁₋₆alkyl, haloC₁₋₆alkoxy, C₂₋₆alkenyl, —CO(O)—C₁₋₆alkyl, —OC(O)—C₁₋₆alkyl or —OC(O)—C₆₋₁₄aryl.

According to yet another embodiment, specifically provided are compounds of formula (I), in which R¹ is hydrogen, methyl, ethyl, cyano, hydroxyl, methoxy, ethoxy F, Cl, CF₃, OCF₃, —CH₂CH═CH₂, —OC(O)—CH₃ or —OC(O)-Ph.

According to one embodiment, specifically provided are compounds of formula (I), in which R² is hydrogen.

According to one embodiment, specifically provided are compounds of formula (I), in which R³ is hydrogen.

According to one embodiment, specifically provided are compounds of formula (I), in which R³ is methyl, ethyl, pentyl or hexyl.

According to yet another embodiment, specifically provided are compounds of formula (I), in which

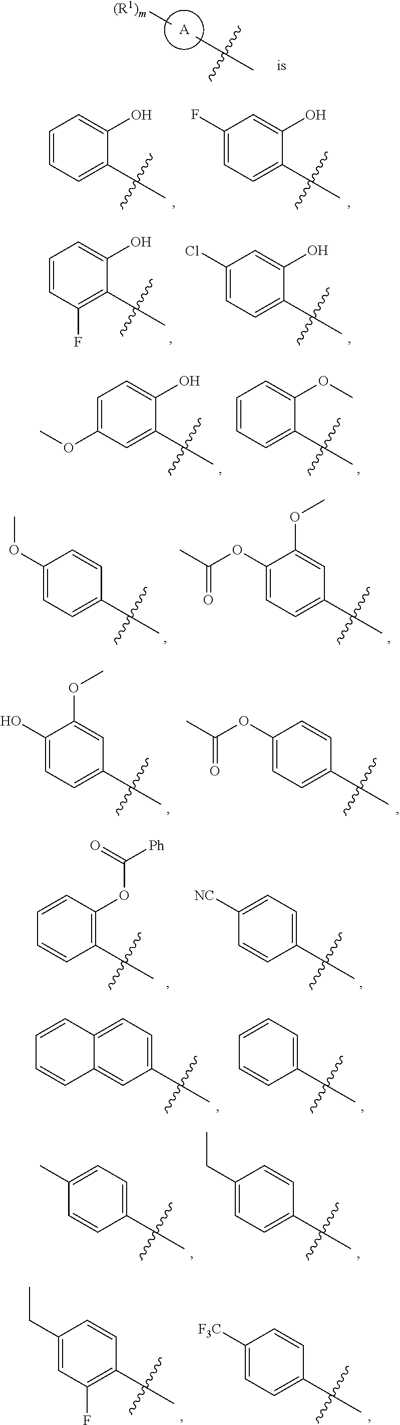

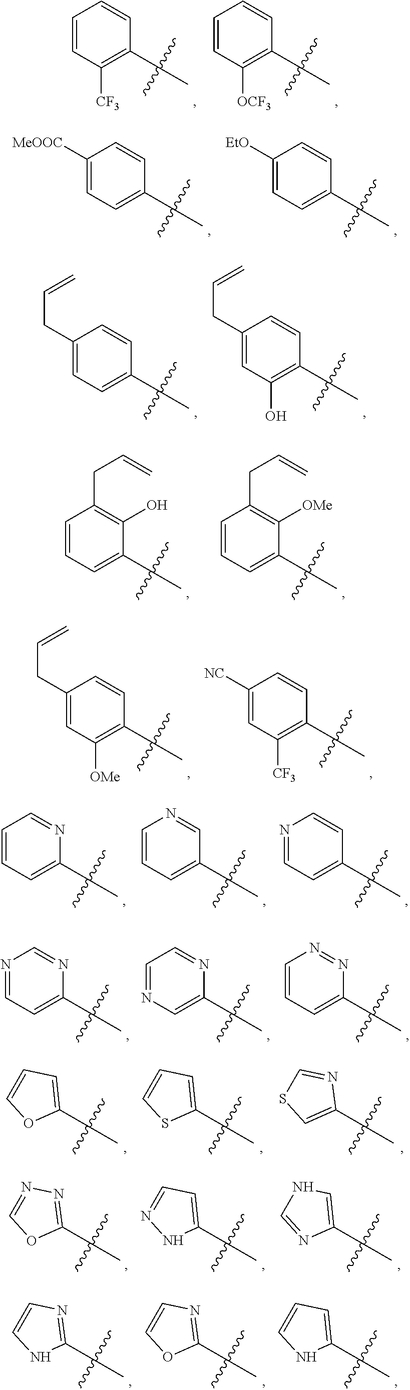

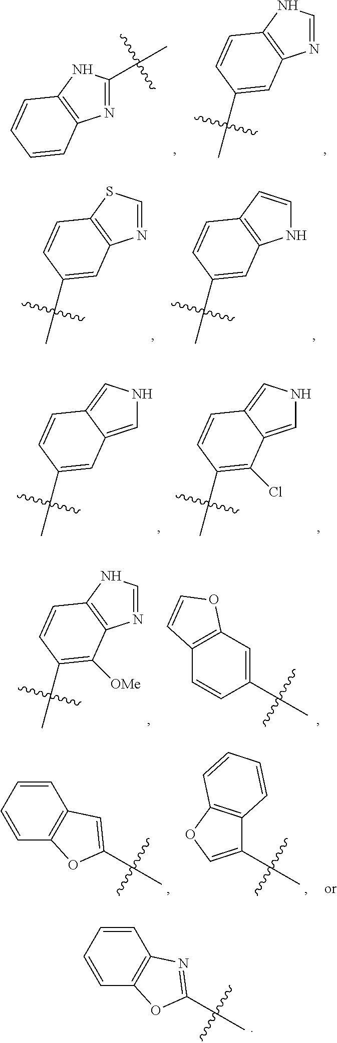

According to yet another embodiment, specifically provided are compounds of formula (I), in which

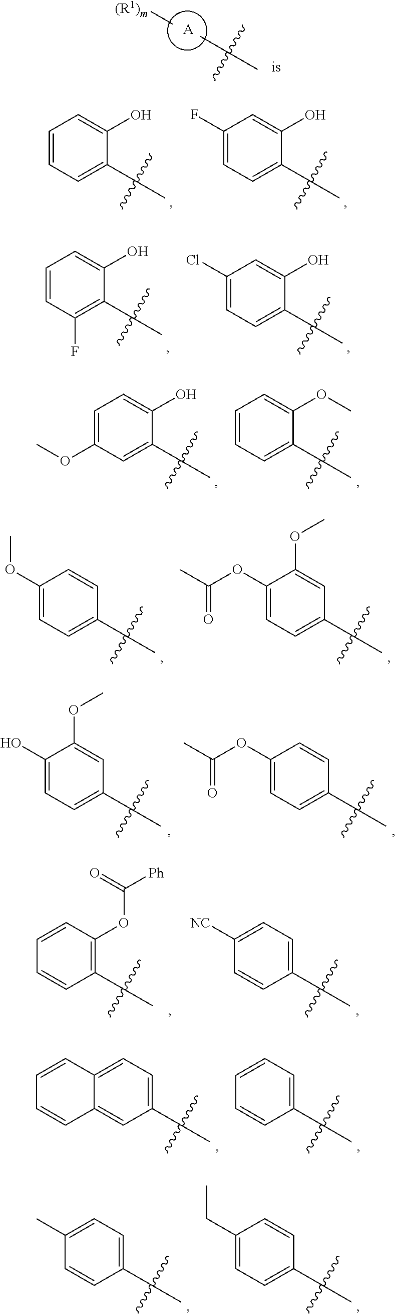

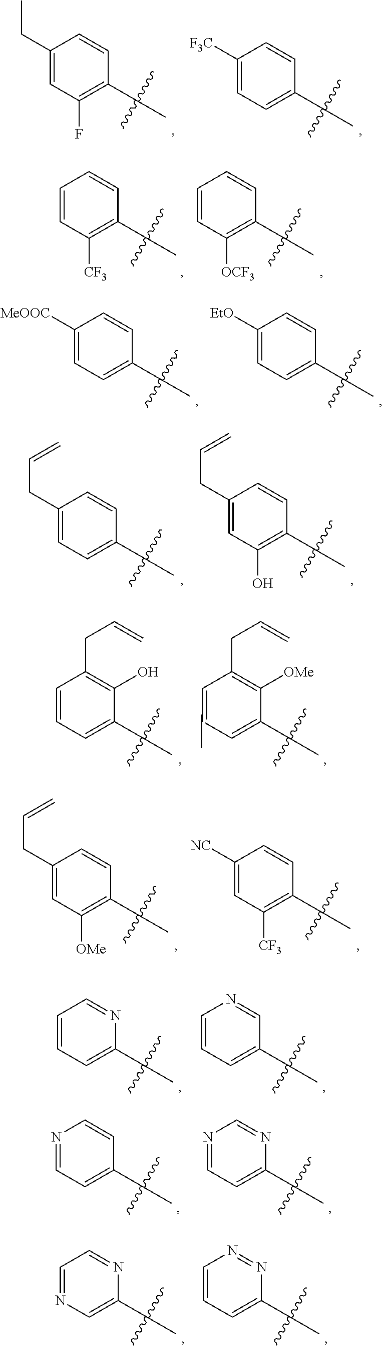

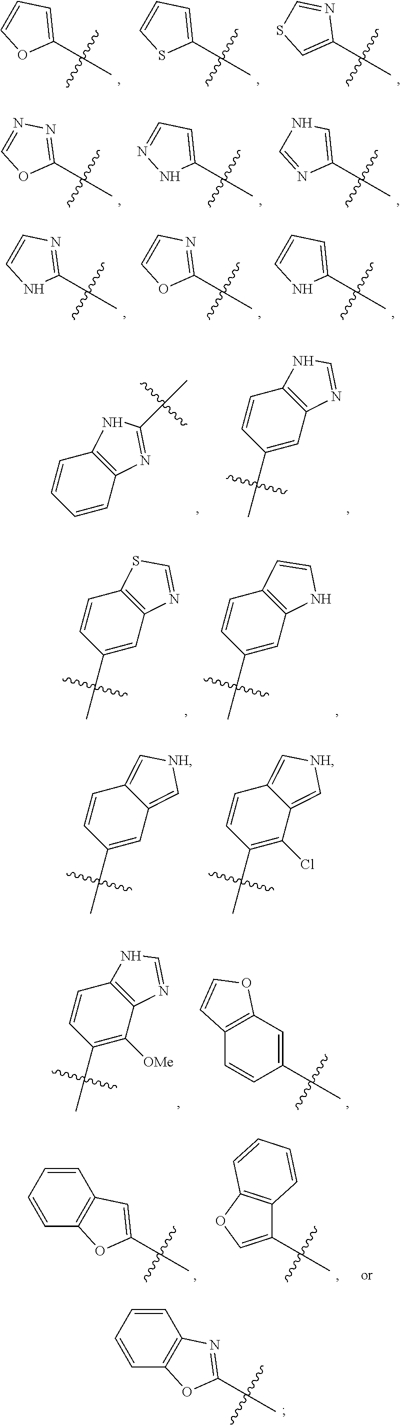

R² is hydrogen; and
R³ is hydrogen, methyl, ethyl, pentyl or hexyl.

In an embodiment, the present invention provides compound of formula:

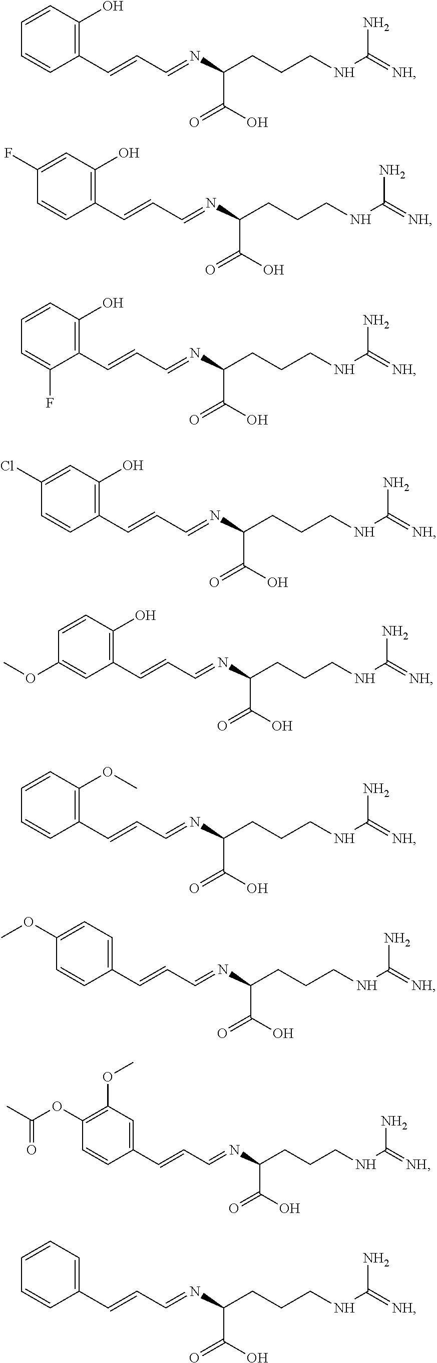

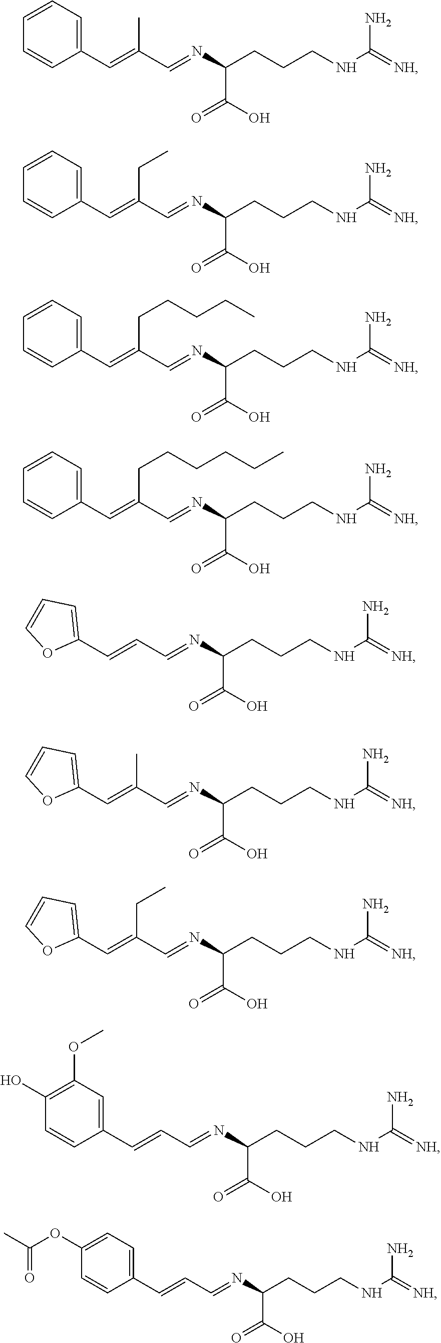

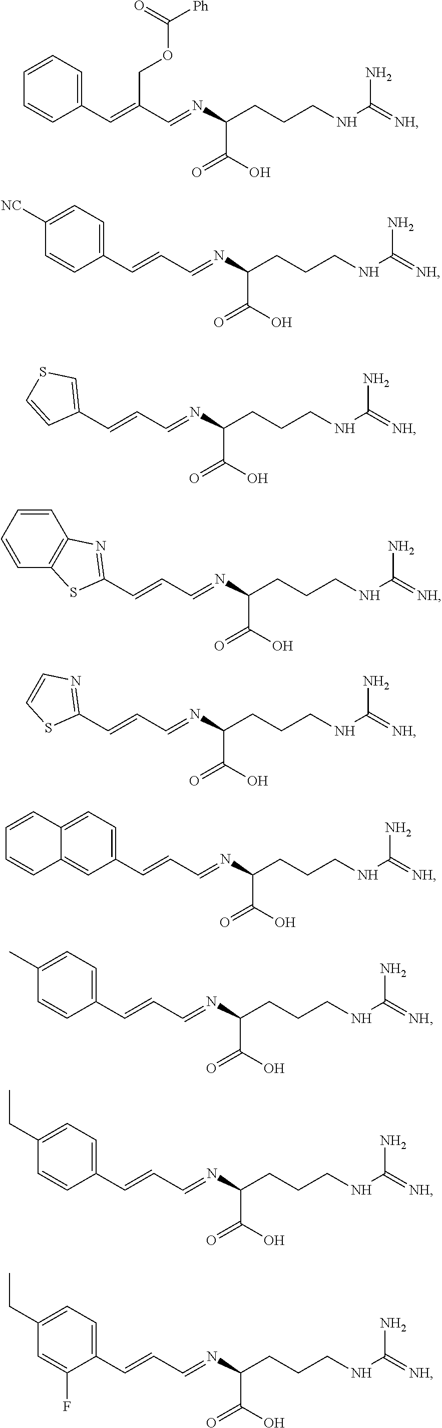

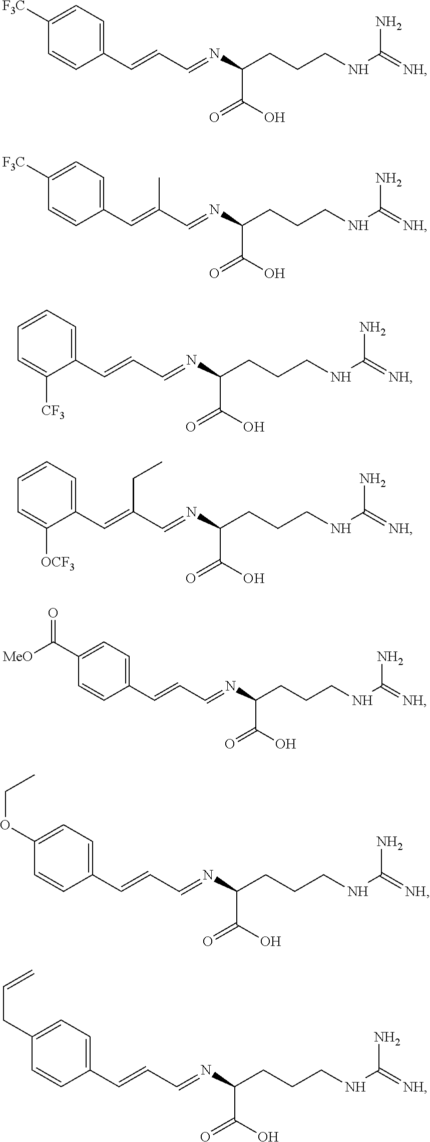

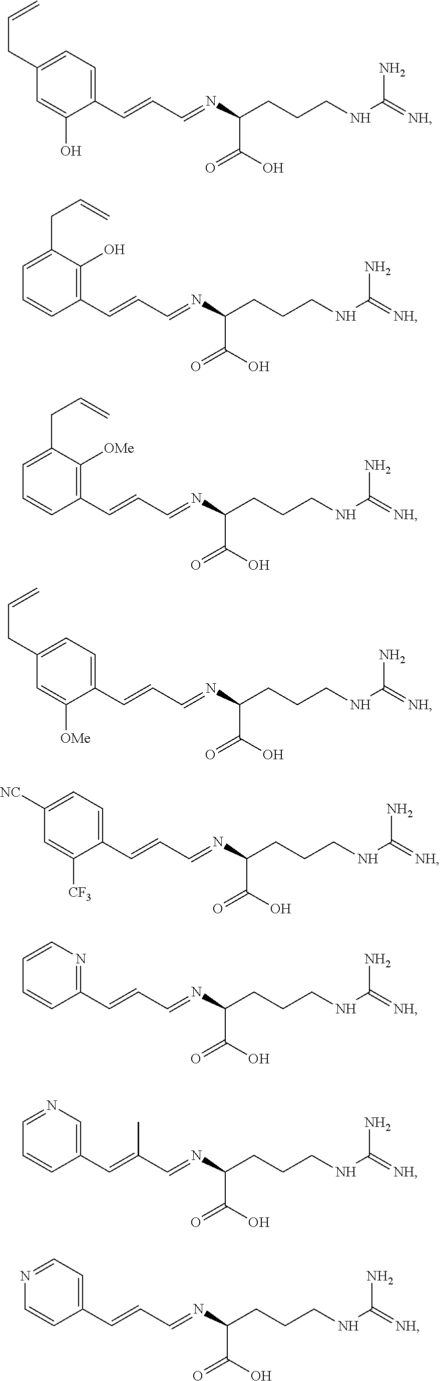

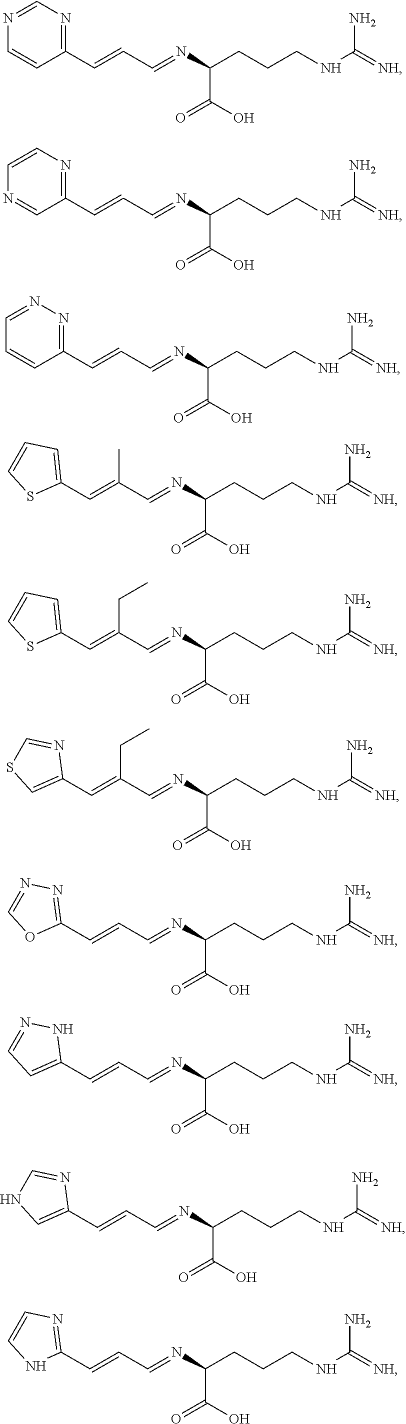

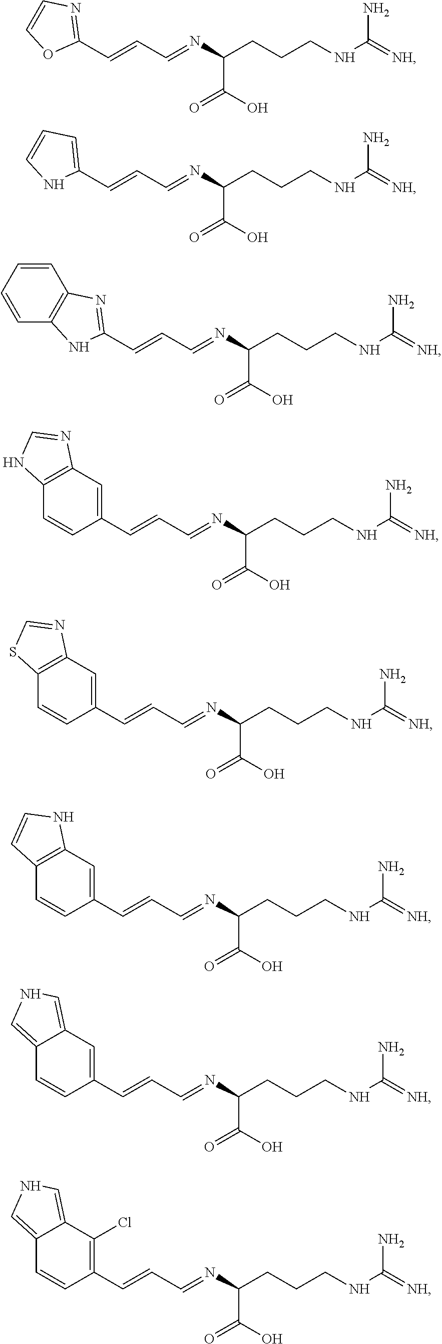

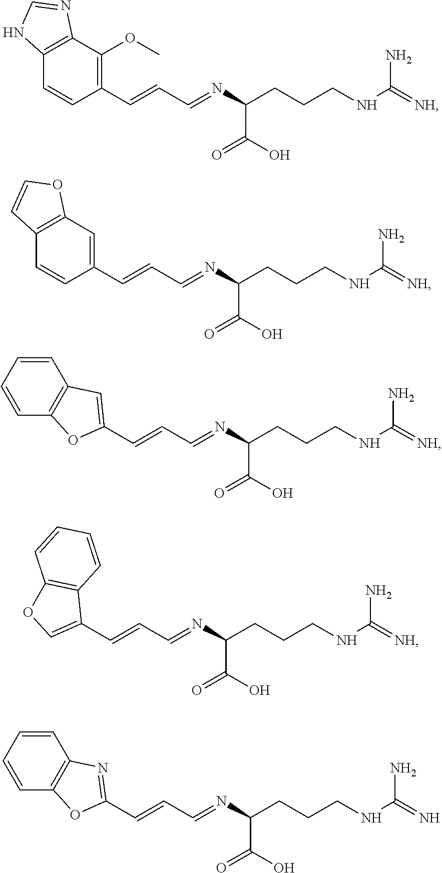 [SMILES: COc1c(/C=C/C=N/[C@@H](CCCNC(=N)N)C(=O)O)ccc2[nH]cnc12 ; N=C(N)NCCC[C@H](/N=C/C=C/c1ccc2ccoc2c1)C(=O)O ; N=C(N)NCCC[C@H](/N=C/C=C/c1cc2ccccc2o1)C(=O)O ; N=C(N)NCCC[C@H](/N=C/C=C/c1coc2ccccc12)C(=O)O ; N=C(N)NCCC[C@H](/N=C/C=C/c1nc2ccccc2o1)C(=O)O]

or agrochemically acceptable salt, or N-oxide thereof.

In an embodiment, the present invention provides a compound of formula:

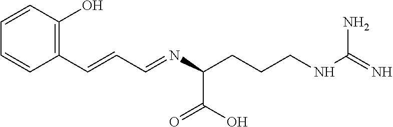 [SMILES: N=C(N)NCCC[C@H](/N=C/C=C/c1ccccc1O)C(=O)O]

or agrochemically acceptable salt thereof or N-oxide thereof.

In an embodiment, the present invention provides a compound of formula:

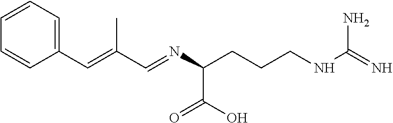 [SMILES: CC(/C=N/[C@@H](CCCNC(=N)N)C(=O)O)=C\c1ccccc1]

or agrochemically acceptable salt thereof or N-oxide thereof.

In an embodiment, the present invention provides a compound of formula:

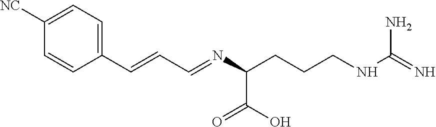 [SMILES: N#Cc1ccc(/C=C/C=N/[C@@H](CCCNC(=N)N)C(=O)O)cc1]

or agrochemically acceptable salt thereof or N-oxide thereof.

In an embodiment, the present invention provides a compound of formula:

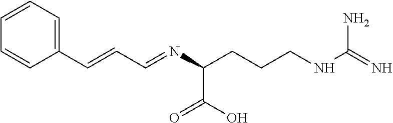 [SMILES: N=C(N)NCCC[C@H](/N=C/C=C/c1ccccc1)C(=O)O]

or agrochemically acceptable salt thereof or N-oxide thereof.

In an embodiment, the present invention provides a compound of formula:

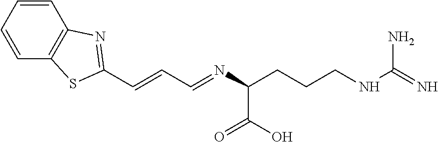 [SMILES: N=C(N)NCCC[C@H](/N=C/C=C/c1nc2ccccc2s1)C(=O)O]

or agrochemically acceptable salt thereof or N-oxide thereof.

It should be understood that unless expressly stated to the contrary, “a compound of general formula (I)” refers to and includes any and all compounds described by formula (I), its embodiments, inclusive of all stereoisomers, geometrical thereof. It should also be noted that the singular forms “a” “an” and “the” include plural reference unless the context clearly dictates otherwise. The examples given in the definitions are non-exhaustive and must not be construed as limiting the present disclosure. It is understood that a substituent should comply with chemical bonding rules and steric compatibility constraints in relation to the particular molecule to which it is attached.

The compounds of the present invention or salts there or N-oxide thereof are suitably used on their own but will generally be administered in the form of a composition in which one or more compounds are suitably formulated into compositions, usually using at least one agrochemically acceptable excipient. Accordingly, the present invention also includes a composition for treating or controlling the parasitic nematode or the nematode infection in a plant comprising an effective amount of one or more compounds of the present invention. In some embodiments, one or more compounds of the present invention are present in an amount that is effective to treat or prevent a nematode infection or a disease, or a disorder, or a condition arising from a nematode infection in a plant.

In an embodiment, the present invention provides an agricultural composition comprising compound of formula (I),

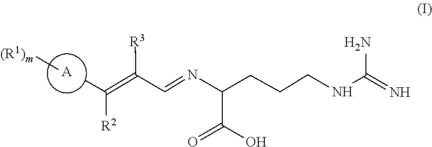

or agrochemically acceptable salt, stereoisomer, enantiomer, tautomer, or N-oxide thereof, and at least one agrochemically acceptable excipient,
wherein,
oxazolyl, isoxazolyl, oxadiazolyl, pyrrolyl, pyrazolyl, imidazolyl, pyrimidinyl, indolyl, isoindolyl, indazolyl, benzofuryl, benzoxazolyl, and benzothiozolyl;
at each occurrence, R¹ is selected from hydrogen, halogen, —OH, —CN, C₁₋₆alkyl, C₁₋₆alkoxy, haloC₁₋₆alkyl, haloC₁₋₆alkoxy, C₂₋₆alkenyl, —CO(O)—C₁₋₆alkyl, —OC(O)—C₁₋₆alkyl and —OC(O)—C₆₋₁₄aryl;

R² and R³ is independently hydrogen, or C₁₋₆alkyl; and
‘m’ is an integer ranging from 0 to 4, both inclusive.

In an embodiment, the present invention provides an agricultural composition comprising compound of formula (I),

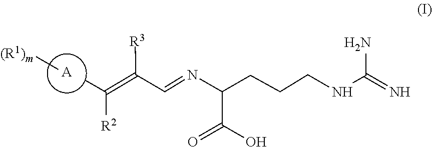

or agrochemically acceptable salt, stereoisomer, enantiomer, tautomer, or N-oxide thereof, and at least one agrochemically acceptable excipient,
wherein,

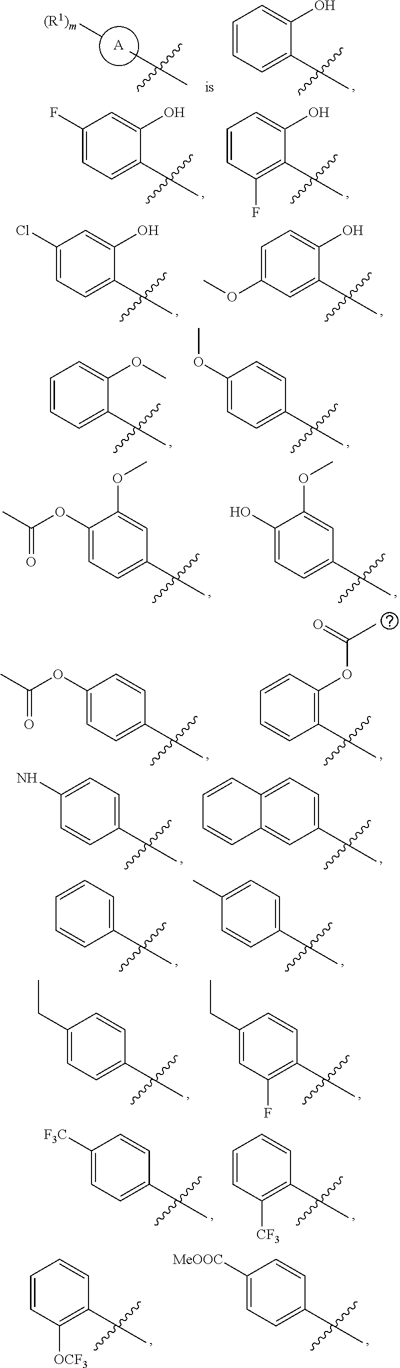

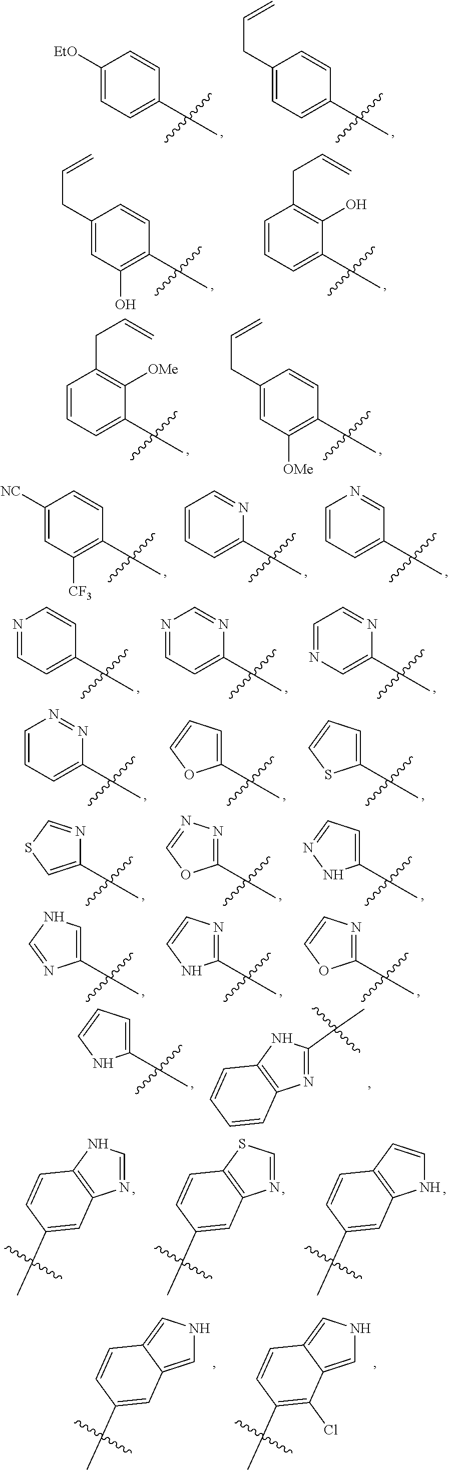

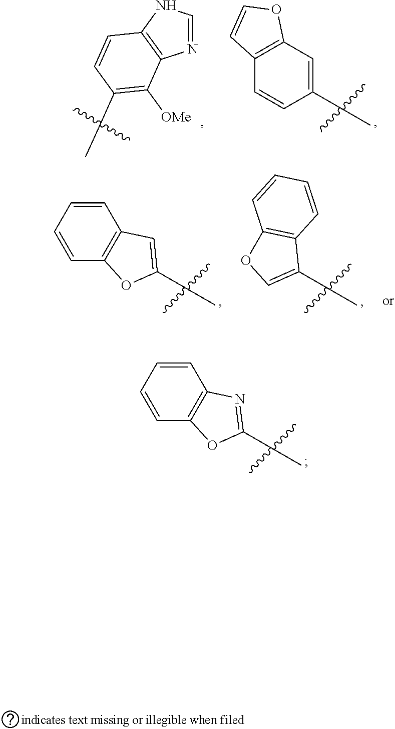

R² is hydrogen; and
R³ is hydrogen, methyl, ethyl, pentyl or hexyl.

In an embodiment, the present invention provides an agricultural composition comprising compound of formula:

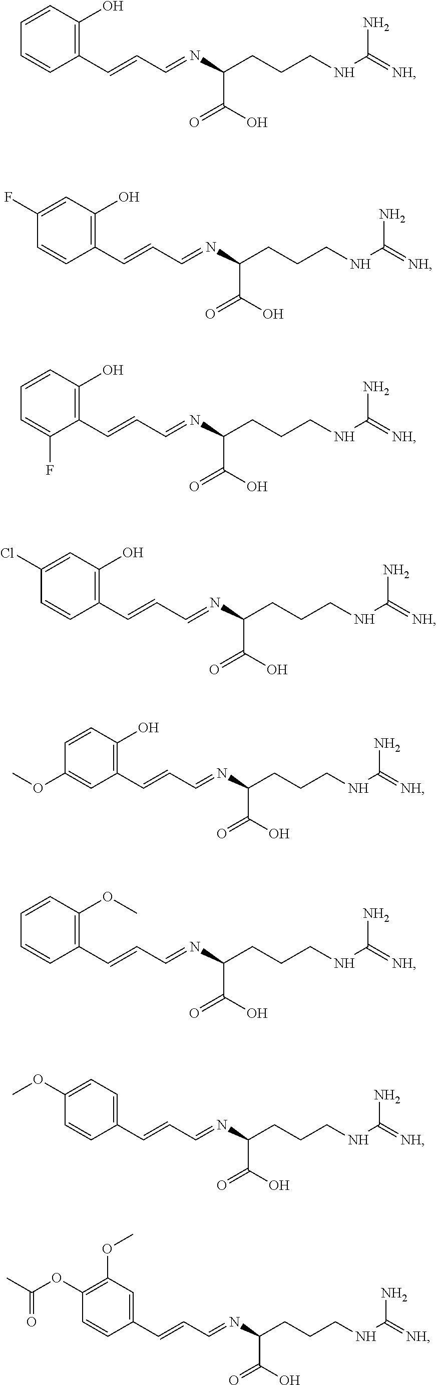

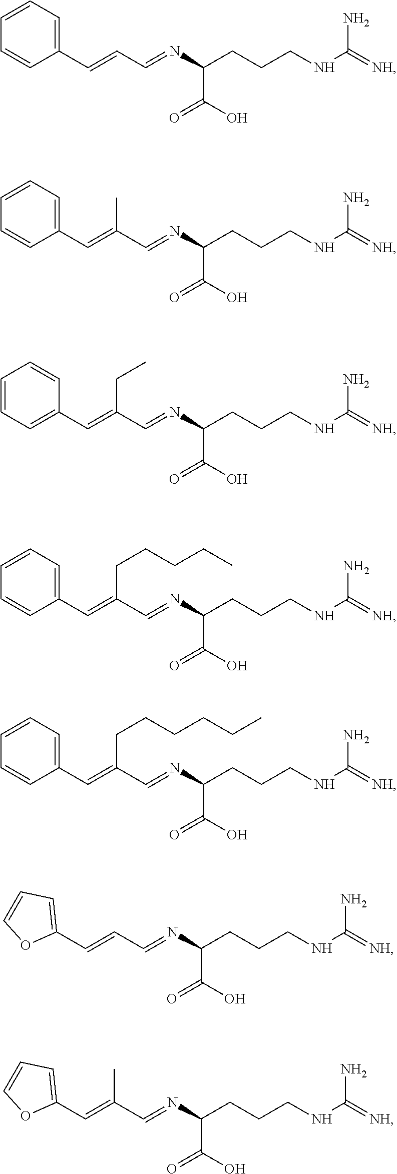

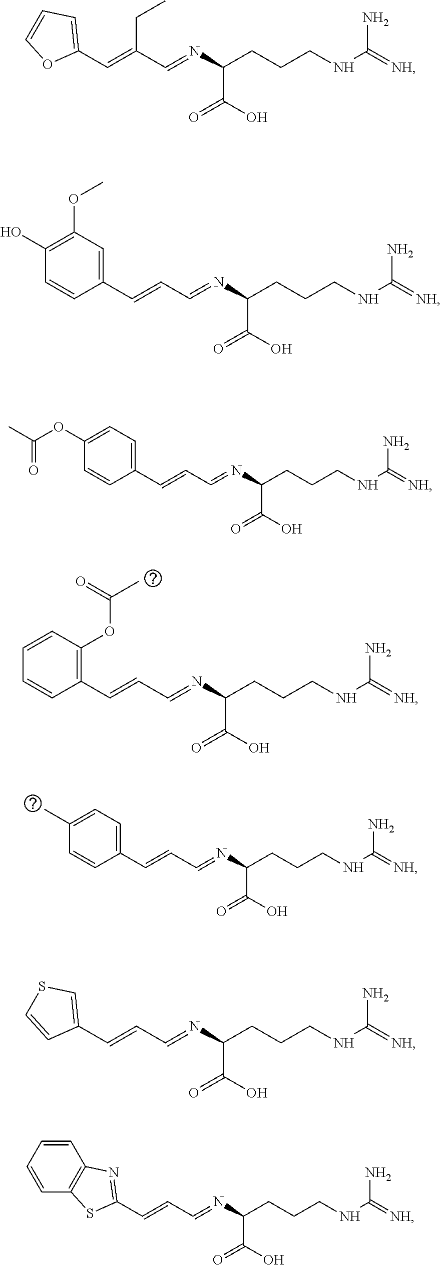

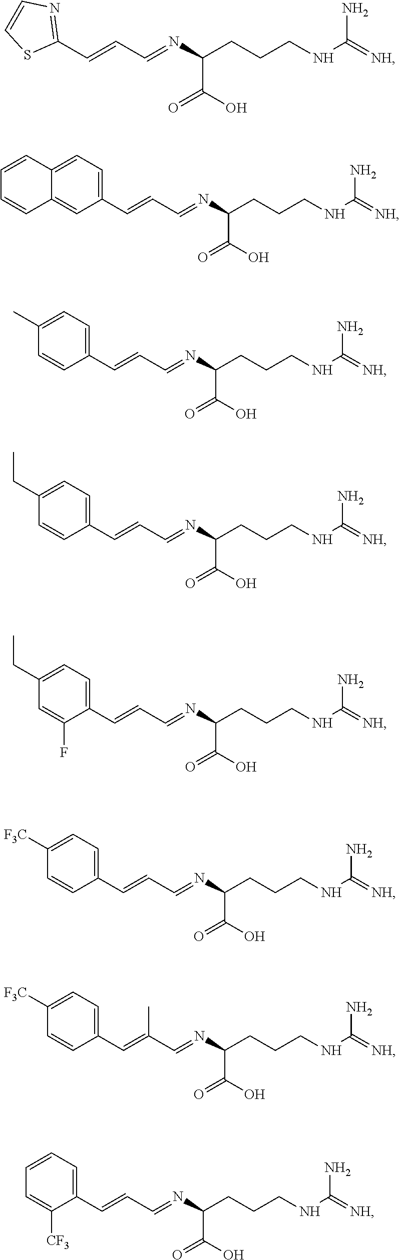

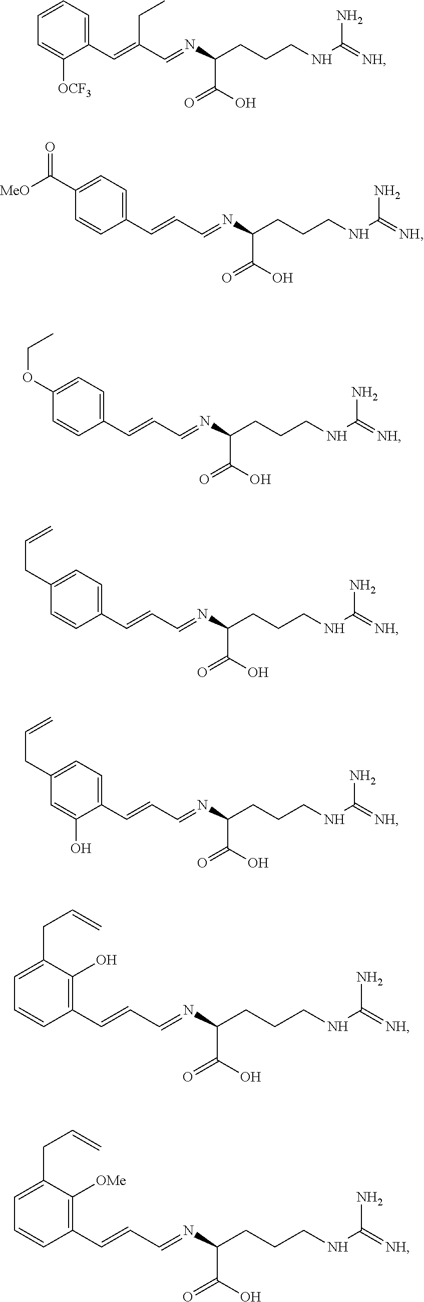

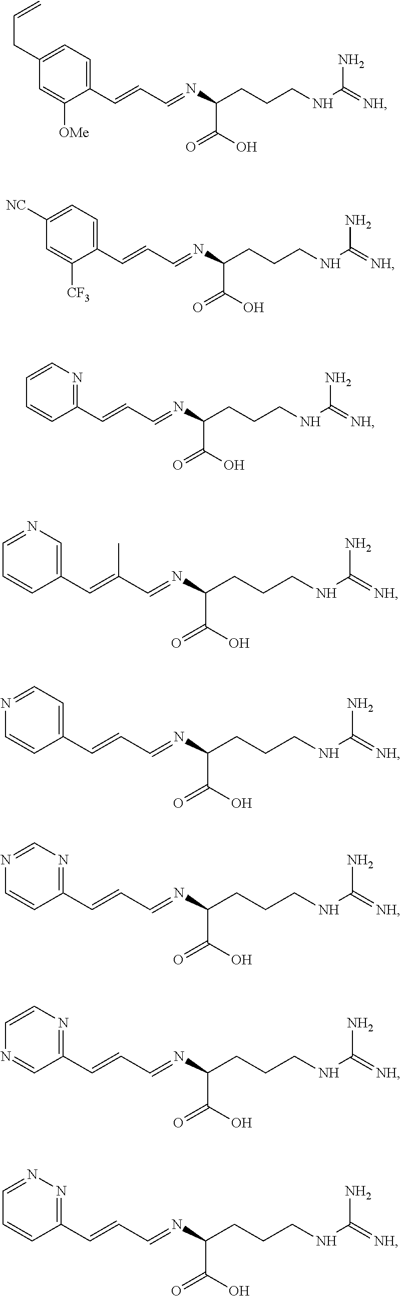

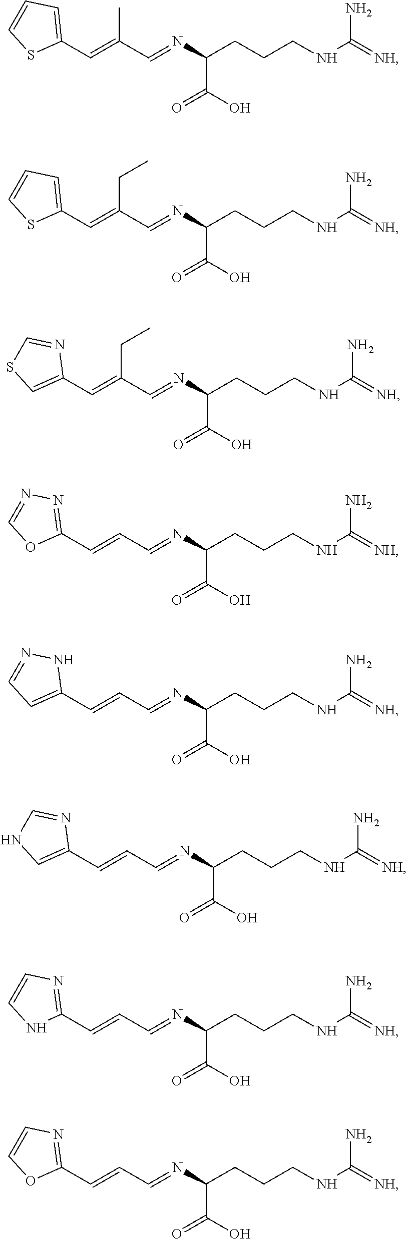

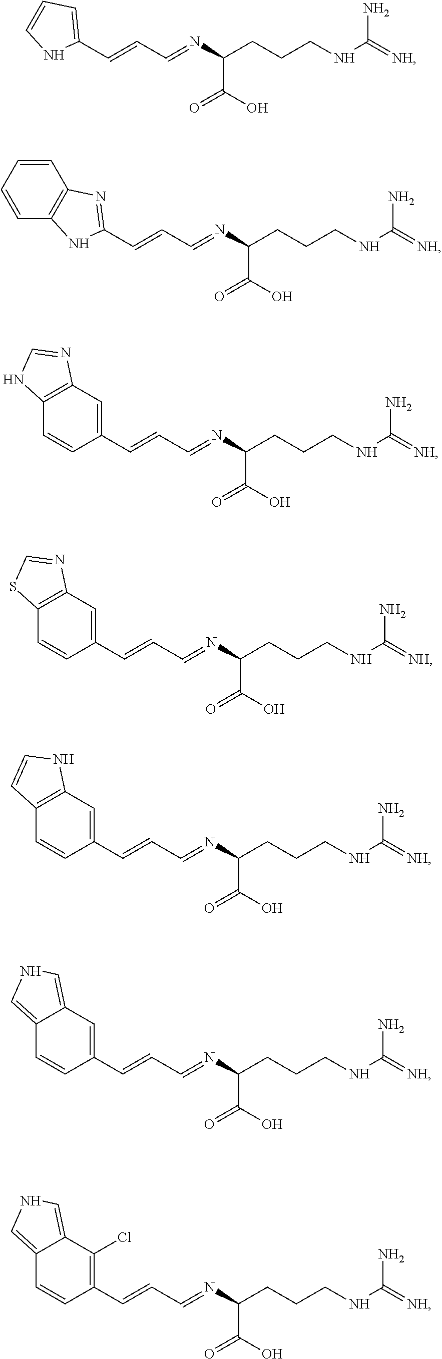

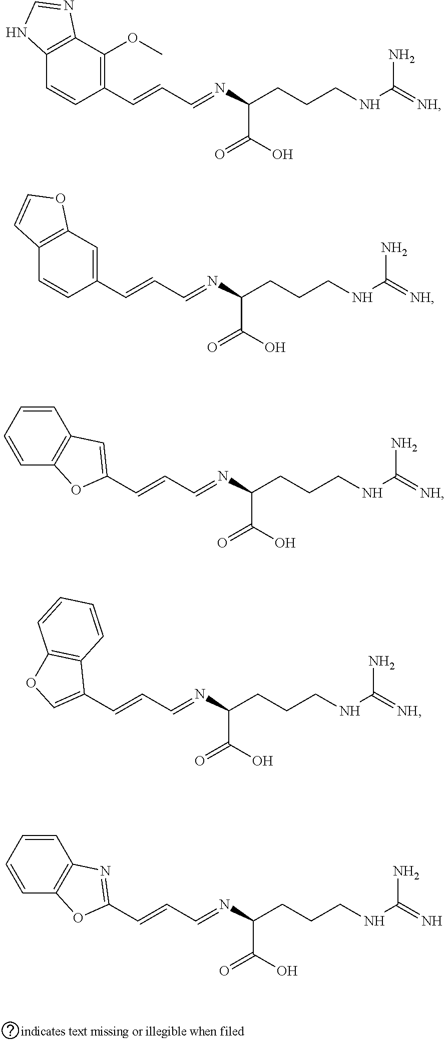 [SMILES: COc1c(/C=C/C=N/[C@@H](CCCNC(=N)N)C(=O)O)ccc2[nH]cnc12 ; N=C(N)NCCC[C@H](/N=C/C=C/c1ccc2ccoc2c1)C(=O)O ; N=C(N)NCCC[C@H](/N=C/C=C/c1cc2ccccc2o1)C(=O)O ; N=C(N)NCCC[C@H](/N=C/C=C/c1coc2ccccc12)C(=O)O ; N=C(N)NCCC[C@H](/N=C/C=C/c1nc2ccccc2o1)C(=O)O]

or agrochemically acceptable salt thereof or N-oxide thereof and at least one agrochemically acceptable excipient.

In an embodiment, the present invention provides an agricultural composition comprising a compound of formula:

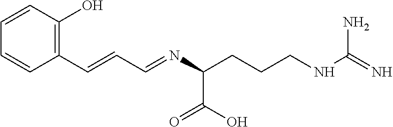 [SMILES: N=C(N)NCCC[C@H](/N=C/C=C/c1ccccc1O)C(=O)O]

or agrochemically acceptable salt thereof or N-oxide thereof and at least one agrochemically acceptable excipient.

In an embodiment, the present invention provides an agricultural composition comprising a compound of formula:

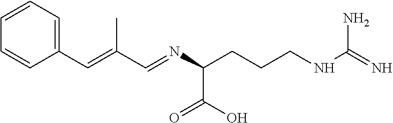 [SMILES: CC(/C=N/[C@@H](CCCNC(=N)N)C(=O)O)=C\c1ccccc1]

or agrochemically acceptable salt thereof or N-oxide thereof and at least one agrochemically acceptable excipient.

In an embodiment, the present invention provides an agricultural composition comprising a compound of formula:

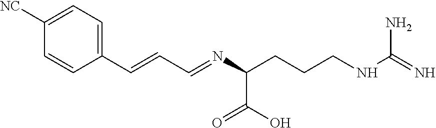 [SMILES: N#Cc1ccc(/C=C/C=N/[C@@H](CCCNC(=N)N)C(=O)O)cc1]

or agrochemically acceptable salt thereof or N-oxide thereof and at least one agrochemically acceptable excipient.

In an embodiment, the present invention provides an agricultural composition comprising a compound of formula:

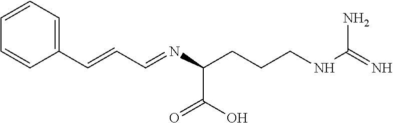 [SMILES: N=C(N)NCCC[C@H](/N=C/C=C/c1ccccc1)C(=O)O]

or agrochemically acceptable salt thereof or N-oxide thereof and at least one agrochemically acceptable excipient.

In an embodiment, the present invention provides an agricultural composition comprising a compound of formula:

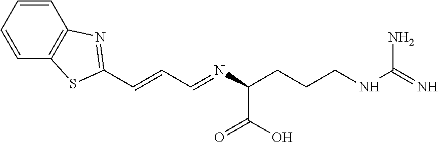 [SMILES: N=C(N)NCCC[C@H](/N=C/C=C/c1nc2ccccc2s1)C(=O)O]

or agrochemically acceptable salt thereof or N-oxide thereof and at least one agrochemically acceptable excipient.

According to an embodiment, the agricultural composition comprises one or more of agrochemically acceptable excipient selected from one or more of surfactants, disintegrating agents, fillers or carriers or diluents, spreading agents, colorants, anticaking agents, binders, buffers or pH adjusters or neutralizing agents, pigments, stabilizers, antifoaming agents or defoamers, penetrants, structuring agents, humectants, sticking agents, anti-freezing agent or freeze point depressants, chelating or complexing or sequestering agents preservatives. However, those skilled in the art will appreciate that it is possible to utilize additional agrochemically acceptable excipients without departing from the scope of the present invention. The agrochemically acceptable excipients are commercially manufactured and available through various companies.

According to an embodiment, the surfactants that are used in the composition of the present invention include one or more of emulsifiers, wetting agents, and dispersing agents. According to an embodiment, the surfactants that are used in the composition include one or more of anionic, non-ionic, and polymeric surfactants.

The anionic surfactants include one or more of, but not limited to a salt of Fatty Acid, a Polycarboxylate, Alkyl Ether Sulfates, an Alkyl Sulfate, an Alkylarylsulfate, an Alkylaryl Sulfonate, an Aryl Sulfonate, a Lignin Sulfonate, an Alkyl Diphenyl Ether Disulfonate, a Polystyrene Sulfonate, a Salt of Alkylphosphoric Acid Ester, an Alkylaryl Phosphate, a Styrylaryl Phosphate, a Salt Of Polyoxyethylene Alkyl Ether Sulfuric Acid Ester, Alpha Olefin Sulfonate Sodium Salt, Alkyl Benzene Sulfonate or Its Salts, Sodium Lauroylsarcosinate, Sulfosuccinates, Polyacrylates, Alkyl Ether Phosphate, a Salt of Polyoxyethylenealkylaryl Phosphoric Acid Ester, Sulfosuccinates-Mono and other Diesters, Phosphate Esters, Alkyl Naphthalene Sulfonate-Isopropyl and Butyl Derivatives; Alkyl Aryl Ether Phosphates, a salt of Polyoxyethylene Aryl Ether Phosphoric Acid Ester, Mono-Alkyl Sulphosuccinates, Aromatic Hydrocarbon Sulphonates, Ammonium Laurylsulphate, Soap, Soap Substitute, Sodium Alkyl Sulfate, Sodium Dodecyl Sulfate, Sodium Dodecylbenzenesulfonate, Sodium Laurate, Sodium Laurethsulfate, Sodium Nonanoyloxybenzenesulfonate, Alkyl Carboxylates, Sodium Stearate, Alpha Olefin Sulphonates, Naphthalene Sulfonate Salts, Alkyl Naphthalene Sulfonate Fatty Acid salts, Naphthalene Sulfonate Condensates-Sodium salt, Fatty Alcohol Sulphates, Alkyl Naphthalene Sulfonate Condensates-Sodium Salt, A Naphthalene Sulfonic Acid Condensed with Formaldehyde or a Salt of Alkylnaphthalene Sulfonic Acid condensed with Formaldehyde or salts or derivatives thereof. However, those skilled in the art will appreciate that it is possible to utilize different anionic surfactants without departing from the scope of the present invention.

The non-ionic surfactants or polymeric surfactants include one or more of but not limited to Polyol Esters, Polyol Fatty Acid Esters, Ethoxylated and Propoxylated Fatty Alcohols, EO and PO Block Copolymers, Di, Tri-Block Copolymers, Polysorbates, Alkyl Polysaccharides, Polyoxyethylene Glycol, Sorbitan Derivatives, Fatty Acid Esters of Sorbitan (Spans) and Their Ethoxylated Derivatives (Tweens), Cocamide Monoethanolamine (MEA), Decyl, Narrow-Range Ethoxylate, Oleyl Alcohol, PEG-10, Polysorbate, Polysorbate 20, Polysorbate 80, Sorbitan, Sorbitanmonolaurate, Sorbitan monostearate, Sorbitan tristearate, Stearyl Alcohol, Castor Oil Ethoxylate, Polyglycol Ethers, Polyadducts of Ethylene Oxide and Propylene Oxide, Polyoxy Ethylene Sorbitan, Fatty Acid Polyglyceride, Polyoxyethylene Alkyl Ether, Polyoxyethylenealkylaryl Ether, a Polyoxyethylenestyrylaryl Ether, a Polyoxyethylene Glycol Alkyl Ether, Alcohol Ethoxylates-C₆ to C_{16/18} Alcohols, Linear and Branched, Alcohol Alkoxylates-Various Hydrophobes and EO/PO Contents and Ratios, a Polyoxyethylene Hydrogenated Castor Oil, salts or derivatives thereof. However, those skilled in the art will appreciate that it is possible to utilize different non-ionic surfactants or polymeric surfactants without departing from the scope of the present invention.

According to an embodiment, the dispersing agents which are used in the composition include, but not limited to non-ionic dispersants selected from one or more of polyvinyl pyrrolidone, polyvinyl alcohol, polyoxyethylene alkyl ethers, polyoxyethylene alkyl phenyl ether, ethoxylated fatty acids, aliphatic alcohol ethoxylates; alkyl ethoxylates; EO-PO block and graft copolymers; However, those skilled in the art will appreciate that it is possible to utilize different non-ionic dispersants without departing from the scope of the present invention.

According to an embodiment, the dispersing agents which are used in the composition include, but not limited to anionic dispersants selected from one or, tristyrylphenolethoxylate phosphate esters; lignin sulphonates, phenyl naphthalene sulphonates, alkali metal, alkylarylsulfonates, alkylsulfonates, mixture of sodium salt of naphthalene sulphonic acid urea formaldehyde condensate and sodium salt of phenol sulphonic formaldehyde condensate, polycarboxylates, sodium alkyl benzene sulfonates, sodium salts of sulfonated naphthalene, sodium naphthalene sulfonate formaldehyde condensates, condensation products of aryl sulphonic acids and formaldehyde, polyaromatic sulfonates, sodium alkyl aryl sulfonates and kraft lignin. However, those skilled in the art will appreciate that it is possible to utilize different anionic dispersants without departing from the scope of the present invention.

According to an embodiment the wetting agents used in the composition include, but are not limited to one or more of phenol naphthalene sulphonates, alkyl naphthalene sulfonate, sodium alkyl naphthalene sulfonate, naphthalene sulphonate sodium salt, dibutylnaphthalene-sulfonic acid, alkylarylsulfonates, dioctyl sulfosuccinate, polyoxyethoxylated fatty alcohols, alkane sulfonates, alkylbenzene sulfonates, alkyl ether phosphates, alkyl ether sulphates and alkyl sulfosuccinic monoesters, salts, derivatives thereof. However, those skilled in the art will appreciate that it is possible to utilize different wetting agents without departing from the scope of the present invention.

According to an embodiment, the carriers that are used in the composition of the present invention include, but are not limited to one or more of solid carriers or fillers or diluents. According to another embodiment, the carriers include mineral carriers, plant carriers, synthetic carriers, water-soluble carriers. However, those skilled in the art will appreciate that it is possible to utilize different carriers without departing from the scope of the present invention.

The solid carriers include natural minerals like clay such as china clay, acid clay, kaolin such as kaolinite, dickite, nacrite, and synthetic and diatomaceous silicas, micas, such as pyrophyllite, talc, silicas such as cristobalite and quartz, such as attapulgite and sepiolite, vermiculite, laponite, pumice, bauxite, hydrated aluminas, perlite, sodium bicarbonate, limestone, natural and synthetic silicates, silicas, surface-modified silicas, zeolite, diatomaceous earth, loess, mirabilite, white carbon, slaked lime, synthetic silicic acid, starch, modified starch, cellulose, plant carriers such as cellulose, chaff, wheat flour, wood flour, starch, rice bran, wheat bran, and soybean flour, casein sodium, sucrose, salt cake, potassium pyrophosphate, sodium tripolyphosphate or derivatives or mixtures thereof.

According to an embodiment, the antifoaming agents or defoamers which are used in the composition of the present invention include but are not limited to one or more of silica, siloxane, silicone dioxide, polydimethyl siloxane, alkyl polyacrylates, ethylene oxide/propylene oxide copolymers, silicone oils and magnesium stearate or derivatives thereof. Preferred antifoaming agents include silicone emulsions long-chain alcohols, fatty acids, fluoro-organic compounds. However, those skilled in the art will appreciate that it is possible to utilize different antifoaming agents without departing from the scope of the present invention. According to an embodiment, the pH-adjusters or buffers or neutralizing agents that are used in the composition include both acids and bases of the organic or inorganic type and mixtures thereof. According to a further embodiment, pH-adjusters or buffers or neutralizing agents include, but are not limited to one or more of organic acids, inorganic acids, and alkali metal compounds or salts, derivatives thereof. According to an embodiment, the organic acids include, but not limited to one or more of citric, malic, adipic, fumaric, maleic, succinic, and tartaric acids, or salts, derivatives thereof, and the mono-, di-, or tribasic salts of these acids or derivatives thereof. According to an embodiment, the salts of inorganic acids include, but not limited to one or more of alkali metal salts such as, sodium chloride, sodium nitrate, potassium nitrate, sodium sulfate, potassium sulfate, sodium monohydrogen phosphate, potassium monohydrogen phosphate, sodium dihydrogen phosphate, potassium dihydrogen phosphate and the like. Mixtures can also be used to create a pH-adjusters or buffers or neutralizing agents. However, those skilled in the art will appreciate that it is possible to utilize different pH adjusters without departing from the scope of the present invention.

According to an embodiment, the anticaking agents which are used in the composition include, but are not limited to one or more of polysaccharides, fumed and precipitated silica (white carbon), a petroleum resin, Foammaster® Soap L sodium stearate, Brij® 700 polyoxyethylene (100) stearylether, sodium acetate, sodium metasilicate, sodium alkylsulfosuccinates, or derivatives thereof. However, those skilled in the art will appreciate that it is possible to utilize different anticaking agents without departing from the scope of the present invention.

According to an embodiment, the spreading agents which are used in the composition include but are not limited to one or more of copolymer of maleic acid with a styrene compound, a (meth)acrylic acid copolymer, aliphatic alcohols, vegetable oils such as cottonseed or inorganic oils, petroleum distillates, trisiloxanes and modified trisiloxanes, or derivatives thereof. However, those skilled in the art will appreciate that it is possible to utilize different spreading agents without departing from the scope of the present invention.

According to an embodiment, the sticking agents which are used in the composition include, but not limited to one or more of paraffin, a polyamide resin, polyacrylate, polyoxyethylene, wax, latex, polyvinyl pyrrolidone, gums such as xanthan gum, vegetable oils such as cottonseed, or inorganic oils, petroleum distillates, modified trisiloxanes, polyglycol, a synthetic resin emulsion or salts or derivatives thereof. However, those skilled in the art will appreciate that it is possible to utilize different sticking agents without departing from the scope of the present invention.

According to an embodiment, the structuring agents that are used in the composition include, but are not limited to one or more of thickeners, viscosity modifiers, tackifiers, suspension aids, rheological modifiers or anti-settling agents. A structuring agent prevents sedimentation of the active ingredient particles after prolonged storage.

According to an embodiment, the structuring agents which are used in the composition include, but not limited to one or more of polyacrylics, polysaccharides, cellulose derivatives, co-polymers of cellulose derivatives, polyvinyl alcohol and derivatives; clays such as kaolin, smectite, attapulgites and gums such as guar gum, xanthan gum, gelatin, dextrin, fumed silica, mixture of fumed silica and fumed aluminium oxide, swellable polymers, poly(ethylene glycol), stachyose, celluloses such as hemicellulose, carboxymethylcellulose, ethylcellulose, hydroxyethylcellulose, hydroxy-methyl ethyl cellulose, hydroxyl ethyl propyl cellulose, methylhydroxyethylcellulose, methylcellulose; plant starches such as corn starch and potato starch. However, those skilled in the art will appreciate that it is possible to utilize different structuring agents without departing from the scope of the present invention.

Preferred structuring agents include one or more of xanthan gum, aluminium carboxymethyl cellulose, silicate, Hydroxypropyl methylcellulose, methylcellulose, polysaccharide, alkaline earth metal silicate, clays, gelatin, and polyvinyl alcohol.

According to an embodiment, the anti-freezing agents or freezing point depressants used in the composition include, but are not limited to one or more of polyhydric alcohols such as ethylene glycol, diethylene glycol, dipropylene glycol, propylene glycol, glycerol, monohydric or polyhydric alcohols, glycol ethers, glycerol, However, those skilled in the art will appreciate that it is possible to utilize different anti-freezing agents without departing from the scope of the present invention.

According to an embodiment, the chelating or complexing or sequestering agents which are used in the composition include, but not limited to one or more of polycarboxylic acids such as polyacrylic acid and the various hydrolyzed poly(methyl vinyl ether/maleic anhydride); N-hydroxyethyliminodiacetic acid, nitrilotriacetic acid (NTA), N,N,N′,N′-ethylenediaminetetraacetic acid, N-hydroxyethyl-N, N′,N′-ethylenediaminetriacetic acid and N,N,N′,N″,N″-diethylenetriaminepentaacetic acid; α-hydroxy acids, such as citric acid, tartaric acid and gluconic acid; orthophosphates, disodium phosphate, monosodium phosphate; condensed phosphates, such as sodium tripolyphosphate, tetrasodium pyrophosphate, sodium hexametaphosphate and sodium tetrapolyphosphate, ethylene diamine tetraacetic acid (EDTA), diethylenetriaminepentaacetic acid (DTPA), N-hydroxyethyl-ethylenediamine-triacetic acid (HEDTA), ethylenediaminediacetate (EDDA), ethylenediaminedi (o-hydroxyphenylacetic) acid (EDDHA), cyclohexane diamine tetraacetic acid (CDTA), fulvic acid, ulmic acid, nucleic acids, cyclodextrin, humic acid, pyrophosphate. However, those skilled in the art will appreciate that it is possible to utilize different chelating agents without departing from the scope of the present invention.

According to an embodiment, the penetrant which is used in the composition include, but not limited to one or more of alcohol, glycol, glycol ether, ester, amine, alkanolamine, amine oxide, quaternary ammonium compound, triglyceride, fatty acid ester, fatty acid ether, N-methyl pyrrolidone, dimethyl formamide, dimethyl acetamide, or dimethyl sulfoxide, polyoxyethylenetrimethylolpropanemonooleate, polyoxyethylene sorbitan monooleate polyoxyethylenetrimethylolpropanedioleate, polyoxyethylene trimethylol propane trioleate, polyoxyethylene sorbitol hexaoleate. However, those skilled in the art will appreciate that it is possible to utilize different penetrants without departing from the scope of the present invention.

According to an embodiment, the humectant is selected from, but not limited to one or more of polyoxyethylene/polyoxypropylene copolymers, particularly block copolymers. Other humectants are propylene glycol, monoethylene glycol, hexylene glycol, butylene glycol, ethylene glycol, diethylene glycol, poly(ethylene glycol), poly(propylene glycol), glycerol and the like; polyhydric alcohol compounds such as propylene glycol ether, derivatives thereof. However, those skilled in the art will appreciate that it is possible to utilize different humectants without departing from the scope of the present invention.

According to an embodiment, the stabilizers which are used in the agricultural composition include, but not limited to one or more of peroxide compounds such as hydrogen peroxide and organic peroxides, zeolite, antioxidants such as phenol compounds, phosphoric acid compounds, EDTA, sodium sulphites, citric acid, citrates and the like. However, those skilled in the art will appreciate that it is possible to utilize other conventionally known stabilizers without departing from the scope of the present invention.

According to an embodiment, preservative is selected from one or more of formic acid, and derivatives of 2H isothiazol-3-one (so-called isothiazolone derivatives) such as alkylisothiazolones (for example 2-methyl-2H-isothiazol-3-one, MIT; chloro-2-methyl-2H-isothiazol-3-one, CIT), benzoisothiazolones (for example 1,2-benzoisothiazol-3 (2H)-one, BIT, commercially available as Proxel® types from Arch Biocides Ltd.) or 2-methyl-4,5-trimethylene-2H-isothiazol-3-one (MTIT), Proxel® from Arch Biocides Ltd. or Acticide® RS from Thor Chemie and Kathon® MK from Lanxess, Sodium Propinoate, Sodium Benzoate, Propyl Paraben, Propyl Paraben Sodum, Potassium Sorbate, Potassium Benzoate, Phenyl Mercuric Nitrate, Phenyl Etehyl Alcohol, Sodium, Ethylparaben, Methylparaben, Butylparaben, Bezyl Alcohol, Benzothonium Chloride, Cetylpyridinium Chloride, Antioxidants includes but not limited to one or more of imidazole and imidazole derivatives (e.g. urocanic acid), 4,4′-thiobis-6-t-butyl-3-methylphenol, 2,6-di-t-butyl-p-cresol (BHT), penta erythrityl tetrakis[3-(3,5,-di-t-butyl-4-hydroxyphenyl)] propionate; amine antioxidants. However, those skilled in the art will appreciate that it is possible to utilize other conventionally known preservatives without departing from the scope of the present invention.

According to an embodiment, the pigments and colorants are selected from but not limited to synthetic chemicals obtained from various manufacturers. The pigments and colorants can be water soluble or water insoluble, in the form of lakes. Dyes can be solvent dyes, acid dyes or basic dyes. Examples of such products include, but not limited, Unisperse Red 3855, Pigmosol Agro Red 3785, pigment 15. However, those skilled in the art will appreciate that it is possible to utilize other conventionally known pigments and colorants without departing from the scope of the present invention.

According to an embodiment, the disintegrating agents which are used in the agricultural composition include, but not limited to one or more of inorganic water soluble salts e.g. sodium chloride; water soluble organic compounds such as agar, hydroxypropyl starch, carboxymethyl starch ether, tragacanth, cross-linked sodium carboxymethyl cellulose, sodium tripolyphosphate, sodium hexametaphosphate, metal stearates, a cellulose powder, dextrin, methacrylate copolymer, Polyplasdone® XL-10 (crosslinked polyvinylpyrrolidone), poly(vinylpyrrolidone). However, those skilled in the art will appreciate that it is possible to utilize other conventionally known disintegrating agents without departing from the scope of the present invention.

According to an embodiment, the binding agents or binders that are used in the agricultural composition include, but not limited to one or more of maltodextrin, carbohydrates such as monosaccharides, disaccharides, oligosaccharides and polysaccharides, complex organic substance, synthetic organic polymers or derivatives and combinations thereof. However, those skilled in the art will appreciate that it is possible to utilize other conventionally known binding agents without departing from the scope of the present invention.

According to an embodiment, the agrochemically acceptable excipient is present in the range of from 0.1% w/w to 98% w/w of the total composition. According to a further embodiment, the agrochemically acceptable excipient is present in the range of from 0.1% w/w to 95% w/w of the total composition.

According to an embodiment the agricultural composition is in the form of a solid or a liquid or a gel or a paste.

According to an embodiment, the solid agricultural composition can include powder, granules, and dust.

According to an embodiment, the agricultural composition can be in the form of powder including wettable powder, and dispersible powder. According to an embodiment, the composition can be in the form of granules including broadcast granule, water disintegrable granule, spheronised granule, pellets, extruded granules and water dispersible granules.

According to an embodiment, the composition preferably is in the form of water dispersible granules, wettable powder, broadcast granule, water disintegrable granule or spheronised granule.

According to an embodiment, the composition preferably is solid composition which is in the form of granules including spheronised granules, extruded granules, water disintegrable granules, wettable powders, water dispersible granules, dustable powder (DP), powders for dry seed treatment (DS), water disintegrable tablet or Water Dispersible powders for slurry seed treatment (WS).

According to an embodiment, the liquid composition can include suspension, emulsion, liquid suspension, flowable concentrate, emulsifiable concentrate, seed dressing, suspo-emulsion, emulsions in water. In a further embodiment, in the liquid composition, the compound of the invention may be present in a suspended, emulsified or dissolved form. Examples of particular suitable formulation types are solutions, soluble concentrates (e.g. SL, LS), dispersible concentrates (DC), suspensions and suspension concentrates (e.g. SC, Oil dispersion (OD), OF, FS), emulsifiable concentrates (e.g. EC), emulsions (e.g. EW, EO, ES, micro emulsion (ME), and suspo emulsion (SE), suspension concentrate for seed treatment (FS), Ultra-Low-Volume (ULV).

According to an embodiment, the liquid composition can include liquid suspension or suspension concentrate (SC); suspoemulsion (SE), oil dispersion (OD), flowable concentrate (FC), or a suspension concentrate for seed treatment (FS), Ultra-Low-Volume (ULV).

According to an embodiment, the particle size of the composition is in the range of 0.1 micron to 50 microns. According to an embodiment, the composition is in the form of granules which are in the size range of 0.025 to 6 mm.

According to an embodiment, the composition in the form of spheronised granules or broadcast granules or water disintegrable granules or extruded granules, wherein the granules are in the size range of 0.025 to 6 mm.

According to an embodiment, the granules disperse into particles in the size range of 0.1 micron to 50 microns.

In an embodiment, the present invention provides an agricultural composition comprising compound of the present invention in the range of 1% w/w to 95% w/w of the total composition and at least one agrochemically acceptable excipient in the range of 1% w/w to 99% w/w of the total composition, wherein particles are present in the size range of 0.1 micron to 50 microns.

In an embodiment, the present invention provides an agricultural composition comprising compound of the present invention in the range of 1% w/w to 95% w/w of the total composition, at least one agrochemically acceptable excipient in the range of 1% w/w to 99% w/w of the total composition, wherein particles are present in the size range of 0.1 micron to 50 micron and the granules are in the size range of 0.025 to 6 mm.

According to an embodiment, the present invention relates to a process of preparing an agricultural composition of the present invention comprising a compound of the present invention present in the range of 1% w/w to 95% w/w of the total composition and at least one agrochemically acceptable excipient present in the range of 1% w/w to 99% w/w of the total composition.

In an embodiment, the present invention includes a method of treating or controlling the parasitic nematode or the nematode infection in a plant comprising administering one or more compounds of the present invention.

In an embodiment, the present invention includes a method of treating or controlling the parasitic nematode or the nematode infection in a plant comprising administering composition of one or more compounds of the present invention.

In another embodiment, the present invention also includes use of one or more compounds of the present invention for treating or controlling the parasitic nematode or the nematode infection in a plant.

In another embodiment, the present invention also includes use of composition of one or more compounds of the present invention for treating or controlling the parasitic nematode or the nematode infection in a plant.

The present invention also includes use of composition of one or more compounds of the present invention for preparation of a medicament for treating or controlling the parasitic nematode or the nematode infection in a plant.

In an embodiment, the present invention includes a method of treating or controlling the parasitic nematode or the nematode infection in a plant comprising administering compound of formula (I)

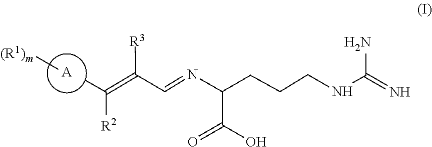

or agrochemically acceptable salt, stereoisomer, enantiomer, tautomer, or N-oxide thereof,
wherein,
ring A is selected from phenyl, naphthyl, pyridinyl, thienyl, furyl, thiazolyl, oxazolyl, isoxazolyl, oxadiazolyl, pyrrolyl, pyrazolyl, imidazolyl, pyrimidinyl, indolyl, isoindolyl, indazolyl, benzofuryl, benzoxazolyl, and benzothiozolyl;
at each occurrence, R¹ is selected from hydrogen, halogen, —OH, —CN, C₁₋₆alkyl, C₁₋₆alkoxy, haloC₁₋₆alkyl, haloC₁₋₆alkoxy, C₂₋₆alkenyl, —CO(O)—C₁₋₆alkyl, —OC(O)—C₁₋₆alkyl and —OC(O)—C₆₋₁₄aryl;
R² and R³ is independently hydrogen, or C₁₋₆alkyl; and
‘m’ is an integer ranging from 0 to 4, both inclusive.

In an embodiment, the present invention includes a method of treating or controlling the parasitic nematode or the nematode infection in a plant comprising administering compound of formula:

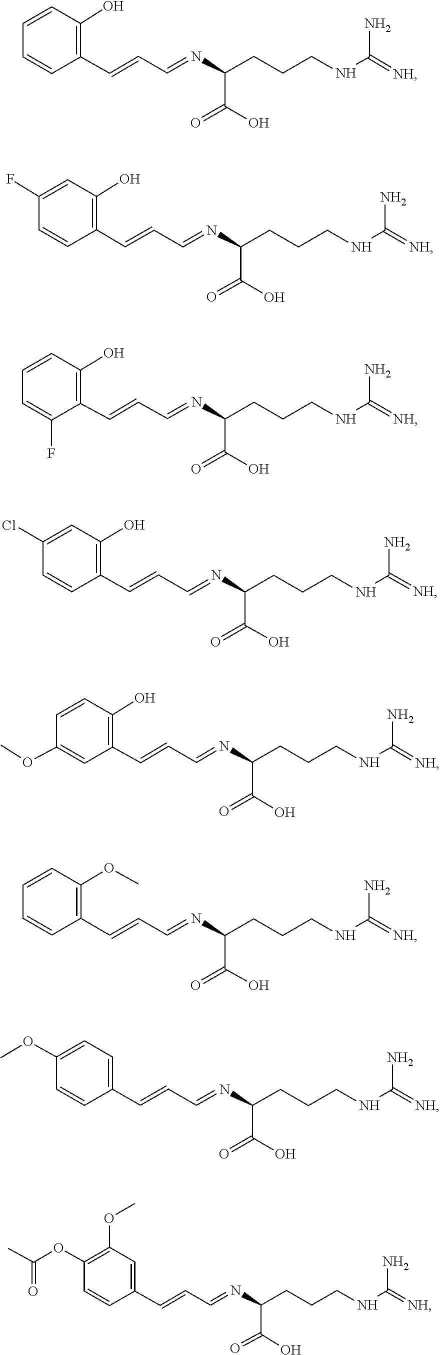

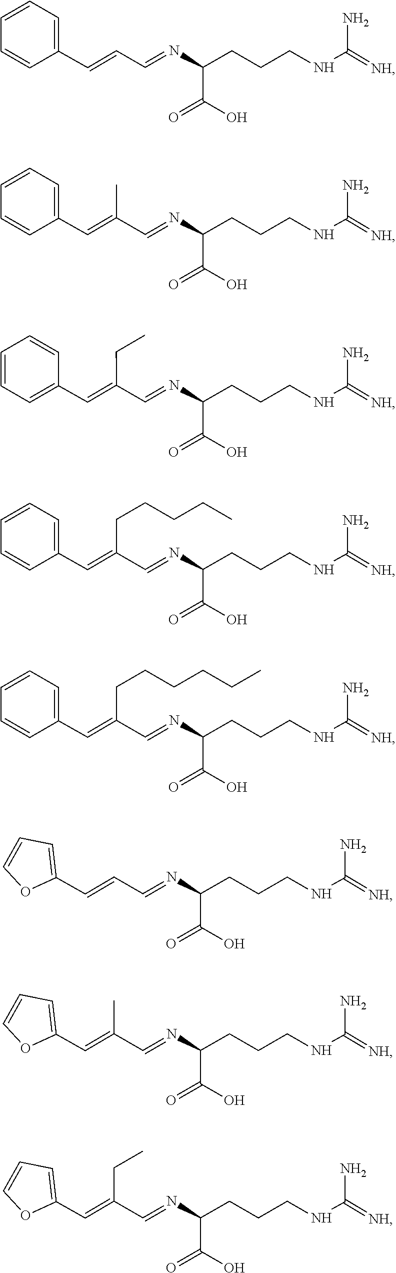

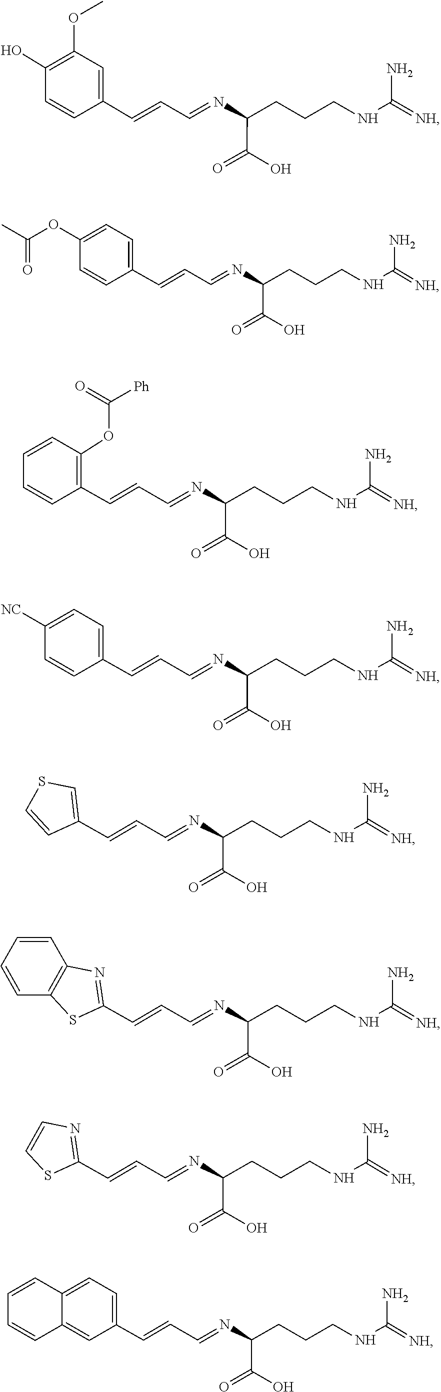

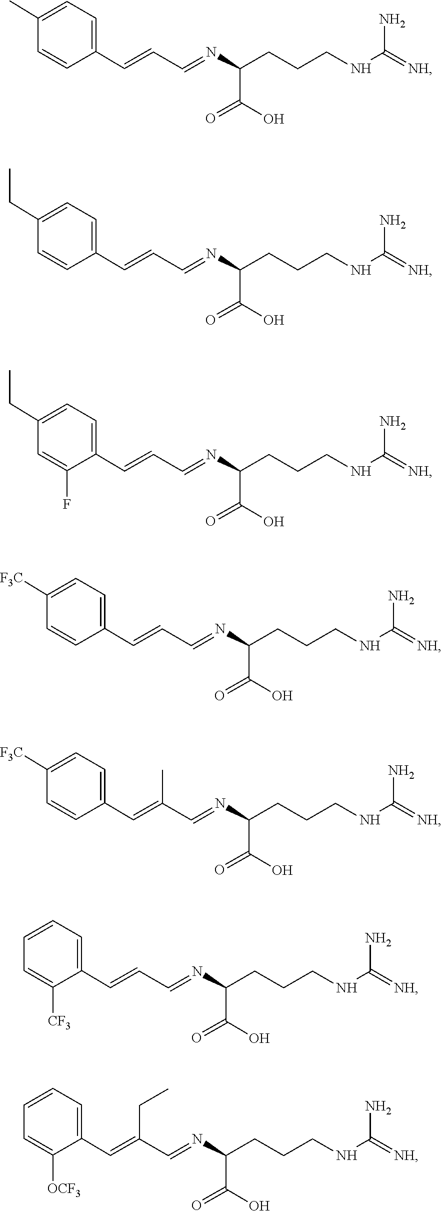

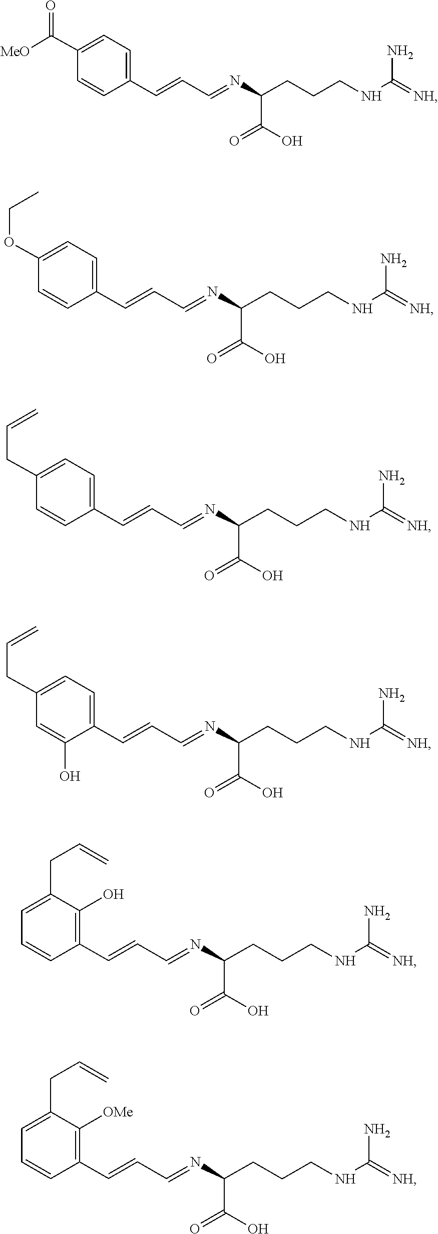 [SMILES: COC(=O)c1ccc(/C=C/C=N/[C@@H](CCCNC(=N)N)C(=O)O)cc1 ; CCOc1ccc(/C=C/C=N/[C@@H](CCCNC(=N)N)C(=O)O)cc1 ; C=CCc1ccc(/C=C/C=N/[C@@H](CCCNC(=N)N)C(=O)O)cc1 ; C=CCc1ccc(/C=C/C=N/[C@@H](CCCNC(=N)N)C(=O)O)c(O)c1 ; C=CCc1cccc(/C=C/C=N/[C@@H](CCCNC(=N)N)C(=O)O)c1O ; C=CCc1cccc(/C=C/C=N/[C@@H](CCCNC(=N)N)C(=O)O)c1OC]

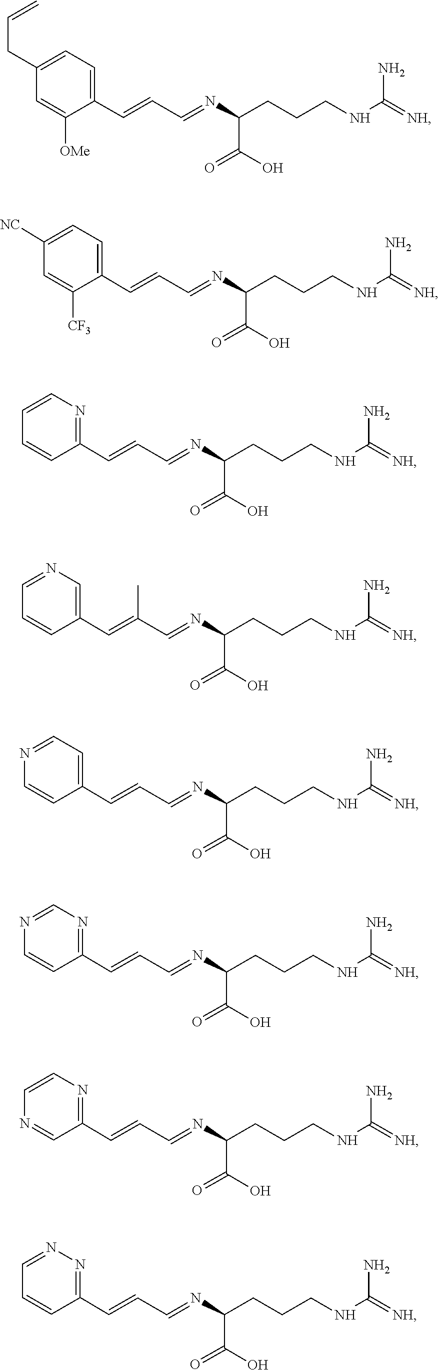

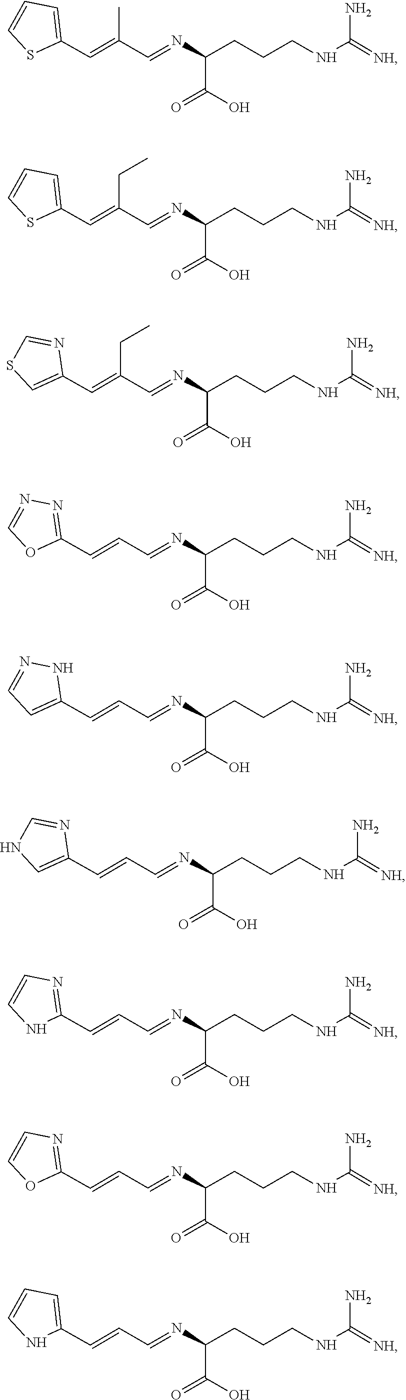

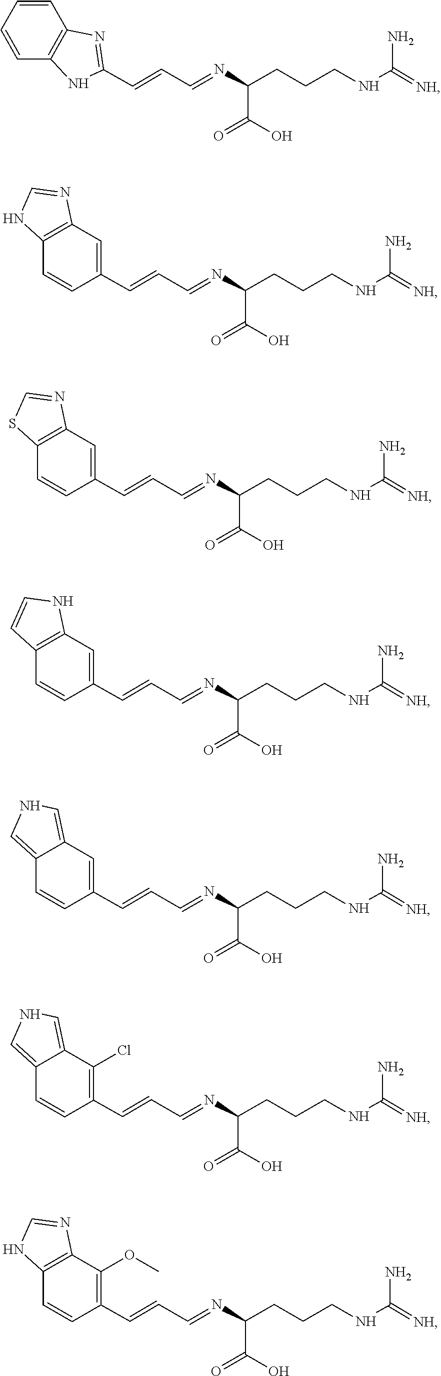

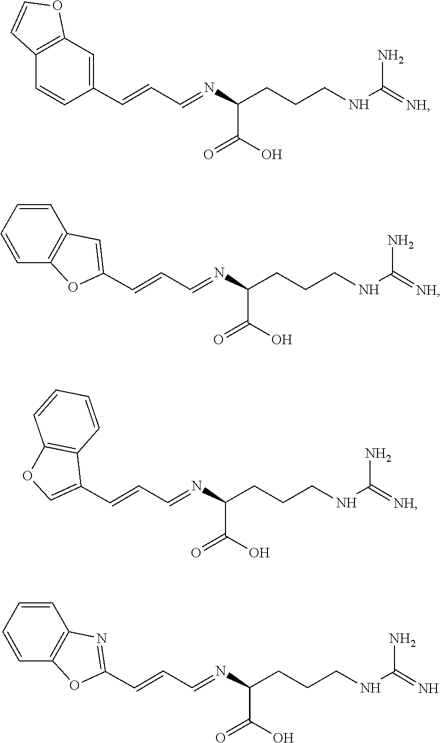 [SMILES: N=C(N)NCCC[C@H](/N=C/C=C/c1ccc2ccoc2c1)C(=O)O ; N=C(N)NCCC[C@H](/N=C/C=C/c1cc2ccccc2o1)C(=O)O ; N=C(N)NCCC[C@H](/N=C/C=C/c1coc2ccccc12)C(=O)O ; N=C(N)NCCC[C@H](/N=C/C=C/c1nc2ccccc2o1)C(=O)O]

or agrochemically acceptable salt thereof or N-oxide thereof.

In an embodiment, the present invention includes a method of treating or controlling the parasitic nematode or the nematode infection in a plant comprising administering compound of formula

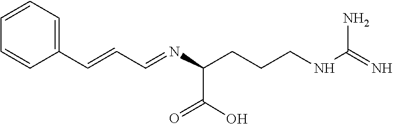 [SMILES: N=C(N)NCCC[C@H](/N=C/C=C/c1ccccc1)C(=O)O]

or agrochemically acceptable salt thereof or N-oxide thereof.

In an embodiment, the present invention includes a method of treating or controlling the parasitic nematode or the nematode infection in a plant comprising administering compound of formula

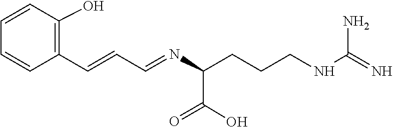 [SMILES: N=C(N)NCCC[C@H](/N=C/C=C/c1ccccc1O)C(=O)O]

or agrochemically acceptable salt thereof or N-oxide thereof.

In an embodiment, the present invention includes a method of treating or controlling the parasitic nematode or the nematode infection in a plant comprising administering compound of formula

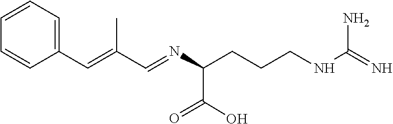 [SMILES: CC(/C=N/[C@@H](CCCNC(=N)N)C(=O)O)=C\c1ccccc1]

or agrochemically acceptable salt thereof or N-oxide thereof.

In an embodiment, the present invention includes a method of treating or controlling the parasitic nematode or the nematode infection in a plant comprising administering compound of formula

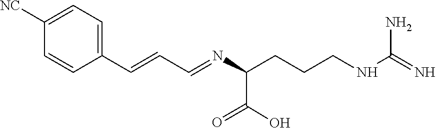 [SMILES: N#Cc1ccc(/C=C/C=N/[C@@H](CCCNC(=N)N)C(=O)O)cc1]

or agrochemically acceptable salt thereof or N-oxide thereof.

In an embodiment, the present invention includes a method of treating or controlling the parasitic nematode or the nematode infection in a plant comprising administering compound of formula

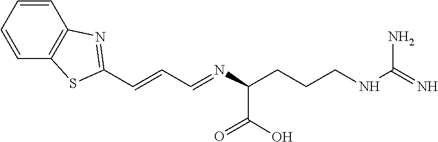 [SMILES: N=C(N)NCCC[C@H](/N=C/C=C/c1nc2ccccc2s1)C(=O)O]

or agrochemically acceptable salt thereof or N-oxide thereof.

In another embodiment, the present invention provides use of an agricultural composition for treating or controlling the parasitic nematode or the nematode infection in a plant wherein the composition comprises compound of formula (I),

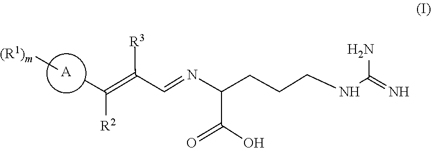

or agrochemically acceptable salt, stereoisomer, enantiomer, tautomer, or N-oxide thereof,
wherein,
ring A is selected from phenyl, naphthyl, pyridinyl, thienyl, furyl, thiazolyl, oxazolyl, isoxazolyl, oxadiazolyl, pyrrolyl, pyrazolyl, imidazolyl, pyrimidinyl, indolyl, isoindolyl, indazolyl, benzofuryl, benzoxazolyl, and benzothiozolyl; at each occurrence, R¹ is selected from hydrogen, halogen, —OH, —CN, C₁₋₆alkyl, C₁₋₆alkoxy, haloC₁₋₆alkyl, haloC₁₋₆alkoxy, C₂₋₆alkenyl, —CO(O)—C₁₋₆alkyl, —OC(O)—C₁₋₆alkyl and —OC(O)—C₆₋₁₄aryl;
R² and R³ is independently hydrogen, or C₁₋₆alkyl; and
‘m’ is an integer ranging from 0 to 4, both inclusive.

In another embodiment, the present invention provides use of an agricultural composition for treating or controlling the parasitic nematode or the nematode infection in a plant wherein the composition comprises compound of formula:

[chemical structure]

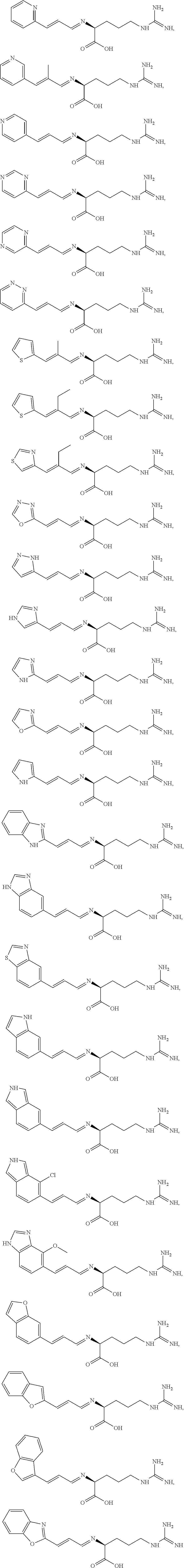

or agrochemically acceptable salt or N-oxide thereof, and at least one agrochemically acceptable excipient.

In another embodiment, the present invention provides use of an agricultural composition for treating or controlling the parasitic nematode or the nematode infection in a plant wherein the composition comprises compound of formula

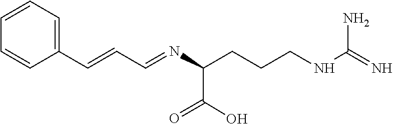 [SMILES: N=C(N)NCCC[C@H](/N=C/C=C/c1ccccc1)C(=O)O]

In another embodiment, the present invention provides use of an agricultural composition for treating or controlling the parasitic nematode or the nematode infection in a plant wherein the composition comprises compound of formula

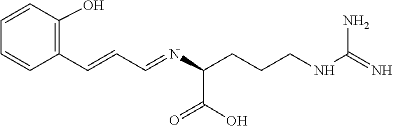 [SMILES: N=C(N)NCCC[C@H](/N=C/C=C/c1ccccc1O)C(=O)O]

or agrochemically acceptable salt or N-oxide thereof, and at least one agrochemically acceptable excipient.

In another embodiment, the present invention provides use of an agricultural composition for treating or controlling the parasitic nematode or the nematode infection in a plant wherein the composition comprises compound of formula

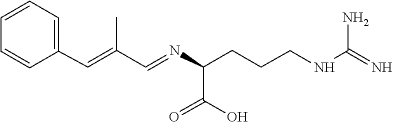 [SMILES: CC(/C=N/[C@@H](CCCNC(=N)N)C(=O)O)=C\c1ccccc1]

or agrochemically acceptable salt or N-oxide thereof, and at least one agrochemically acceptable excipient.

In another embodiment, the present invention provides use of an agricultural composition for treating or controlling the parasitic nematode or the nematode infection in a plant wherein the composition comprises compound of formula

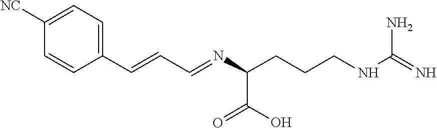 [SMILES: N#Cc1ccc(/C=C/C=N/[C@@H](CCCNC(=N)N)C(=O)O)cc1]

or agrochemically acceptable salt or N-oxide thereof, and at least one agrochemically acceptable excipient.

In another embodiment, the present invention provides use of an agricultural composition for treating or controlling the parasitic nematode or the nematode infection in a plant wherein the composition comprises compound of formula

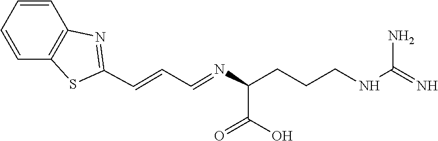 [SMILES: N=C(N)NCCC[C@H](/N=C/C=C/c1nc2ccccc2s1)C(=O)O]

or agrochemically acceptable salt or N-oxide thereof, and at least one agrochemically acceptable excipient.

Nematodes include parasitic nematodes such as root-knot, cyst, and lesion nematodes. The term “nematode” encompasses eggs, larvae, juvenile and mature forms of nematodes. The parasitic nematodes may include but not limited to root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Eelonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

In the context of treating or controlling the parasitic nematode or the nematode infection in a plant, an effective amount of one or more compounds of the present invention, is an amount that, for example, reduces the amount of infection by the nematode in the plant compared to the amount of infection by the nematode in the plant without administration of one or more compounds of the application. Reducing the amount of infection may be assessed, for example, by detecting an amount of viable or living nematodes in the plant, and/or by observing or assessing the extent of a disease, disorder or condition caused by a nematode infection.

The dosage of one or more compounds of the present invention in plants, varies depending on many factors such as the pharmacodynamic properties thereof, the mode of administration, the age, health and weight/mass of the plant, the nature and extent of the symptoms, the frequency of the treatment and the type of concurrent treatment, if any. The one or more compounds of the present invention thereof may be administered initially in a suitable dosage that may be adjusted as required, depending on the response.

In some embodiments, one or more compounds of the present invention are applied to plants at any suitable rate, the selection of which can be made by a person skilled in the art. Factors to consider include, for example, the identity of the plant, the identity of the nematode, the identity of the plant disease, disorder or condition, the severity of the nematode infection, the severity of the plant disease, disorder or condition, the age of the plant, the activity of one or more compounds of the application and the concentration of one or more compounds of the application, or a combination thereof.

In some embodiments, the foliage of the plant and/or the soil surrounding the plant is contacted with one or more compounds of the application.

In some embodiments, the nematode infects plants and one or more compounds are administered to the soil or to plants. In some embodiments, one or more compounds are administered to soil before planting. In some embodiments, one or more compounds are administered to soil after planting. In some embodiments, one or more compounds are administered to soil using a drip system. In some embodiments, one or more compounds are administered to soil using a drench system. In some embodiments, one or more compounds are administered to plant roots or plant foliage (e.g., leaves, stems). In some embodiments one or more compounds are tilled into the soil or administered in furrow. In some embodiments, one or more compounds are administered to seeds. In some embodiments, one or more compounds are applied as a seed coating.

In an embodiment, the present invention also provides a method for protecting a seed from a parasitic nematode comprising contacting the seed with an effective amount of a compound of Formula (I) or salt thereof or N-oxide thereof (e.g., as a composition described herein).

Further, the compositions of the present invention are suitable for drip irrigation or sprinkler irrigation in addition to other methods of applications of the agricultural compositions.

According to an embodiment, specifically provided are compounds of formula (I), exhibit greater than 90% reduction of nematode population compared to the untreated control at concentrations such as 300, 500 or 1000 ppm.

According to an embodiment, specifically provided are compounds of formula (I), exhibit greater than 80% reduction of nematode population compared to the untreated control at concentrations such as 300, 500 or 1000 ppm.

According to an embodiment, specifically provided are compounds of formula (I), exhibit greater than 50% reduction of nematode population compared to the untreated control at concentrations such as 300, 500 or 1000 ppm.

In some embodiments, the nematode infects plants and one or more compositions are administered to the soil or to plants. In some embodiments, one or more compositions are administered to soil before planting. In some embodiments, one or more compositions are administered to soil after planting. In an embodiment, one or more compositions are administered to soil using a drip system. In some embodiments, one or more compositions are administered to soil using a drench system. In some embodiments, one or more compositions are administered to plant roots or plant foliage (e.g., leaves, stems). In some embodiments one or more compositions are tilled into the soil or administered in furrow. In some embodiments, one or more compositions are administered to seeds.

In an embodiment, the methods of the application comprise administering one or more compositions of the application through means selected from but not limited to pre-planting, post-planting, as a feed additive, a drench and an external application.

In an embodiment, the term ‘plant’ refers to all physical parts of a plant, including seeds, seedlings, saplings, roots, tubers, stems, stalks, foliage and fruits.

In an embodiment the plant is a cultivated plant. In some embodiments, the plant is an agricultural crop plant. In a further embodiment, the plant includes, but is not limited to, soybeans, cotton, flax, hemp, jute, corn, tobacco, nuts, almonds, coffee, tea, pepper, grapevines, hops, wheat, barley, rye, oats, rice, maize, sorghum, apples, pears, plums, peaches, banana, plantains, cherries, strawberries, raspberries, blackberries, beans, lentils, peas, soya, oilseed rape, mustard, poppies, olives, sunflowers, coconut, castor, cocoa, ground nuts, spinach, asparagus, lettuce, cabbages, carrots, onions, tomatoes, potatoes, bell peppers, cucumbers, melons, pumpkins, sugar cane, sugar beet, fodder beet, avocado, cinnamon, camphor, oranges, tangerines, lemons, limes, grapefruit, latex plants, ornamental plants, and/or turf grasses.

### Methods of Preparation

The compounds described herein, including compounds of general formula (I) and specific examples, are prepared using techniques known to one skilled in the art through the reaction sequences depicted in Scheme 1. Furthermore, in the scheme, where specific bases, reagents, solvents, etc. are mentioned, it is understood that other suitable bases, reagents, etc. may be used and are included within the scope of the present invention. Modifications to reaction conditions, for example, temperature, duration of the reaction or combinations thereof, are envisioned as part of the present invention. The compounds obtained by using the general reaction sequences may be of insufficient purity. These compounds can be purified by using any of the methods for purification of organic compounds known to a person skilled in the art, for example, crystallization or silica gel or alumina column chromatography using different solvents in suitable ratios. All possible stereoisomers are envisioned within the scope of this invention.

### Scheme 1:

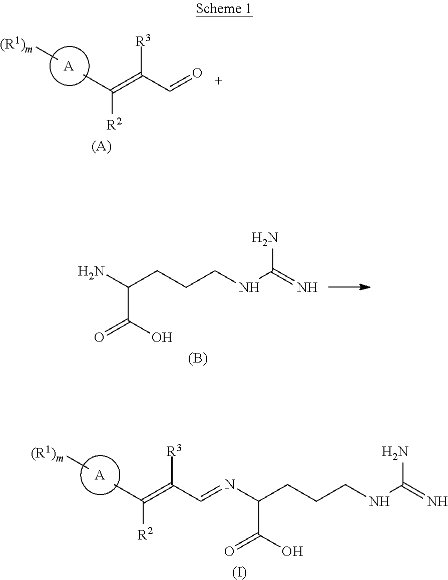

The compounds of the general formula (I) may be prepared as shown in Scheme 1, by reacting the compound of formula (A) or salt thereof with the compound of formula (B) or salt thereof. The reaction can be carried out in the presence of suitable base or it may be carried out in the absence of a base. The reaction may be carried out in the presence of a solvent or mixture of solvents at a suitable temperature range, for example between −10° C. to 100° C. to afford a compound of the general formula (I). The suitable solvents include, but not limited to, water, alcohol (methanol, ethanol, n-propanol, iso-propanol, n-butanol or iso-butanol) tetrahydrofuran, ethyl acetate and acetonitrile or mixture thereof. The suitable bases may include but not limited to organic bases (for example trimethylamine or diisopropylethylamine). The suitable bases may include, but not limited to inorganic bases (for example LiOH, NaOH, NaHCO₃, Na₂CO₃, KOH, KHCO₃, K₂CO₃, Cs₂CO₃ or CsOH).

The compound of Formula (A) may be prepared by as per the process shown in Scheme 2.

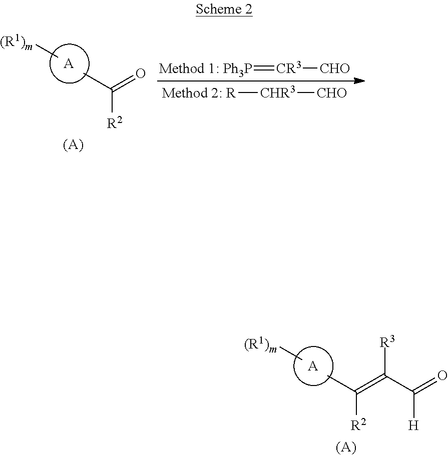

Method-1: The compound of formula (C) is reacted with an appropriate phosphine aldehyde in the presence of a suitable solvent or mixture of solvents (such as one or more of chloroform, dichloromethane, tetrahydrofuran, 1,4-dioxane, acetonitrile, ethyl acetate, xylene and toluene) at, for example, a temperature range such as between 25° C. to 150° C. to afford the compound of formula (A). The obtained product may be further purified using silica gel column chromatography or crystallization using a suitable solvent or mixture of solvents. The suitable solvent may include but not limited to chloroform, dichloromethane, ethyl acetate, methanol, ethanol, acetone or any combination of any of the forgoing.

Method-2: The compound of formula (C) can be reacted with a carbonyl compound containing α-hydrogen(s) in the presence of a suitable base. The suitable bases include, but are not limited to, for example LiOH, NaOH, NaHCO₃, Na₂CO₃, KOH, KHCO₃, K₂CO₃, Cs₂CO₃ and CsOH. The reaction may be carried out in the presence of suitable solvent or mixture of solvents. The suitable solvent or mixture of solvents can be water, methanol, ethanol, n-propanol, iso-propanol, n-butanol, iso-butanol, tetrahydrofuran or any combination of any of the foregoing. The reaction may be carried out at a suitable temperature range such as between −10° C. to 100° C. to afford the desired product. The obtained product may be further purified using silica gel column chromatography or crystallization using suitable solvents or mixture of solvents which may include chloroform, dichloromethane, ethyl acetate, methanol, ethanol, acetone or any combination of any of the foregoing.

While the present invention has been described in terms of its specific embodiments, certain modifications and equivalents will be apparent to those skilled in the art and are intended to be included within the scope of the present invention. In the following section, aspects are described by way of examples to illustrate the processes of the invention. However, these do not limit the scope of the present invention. Several variants of these examples would be evident to person ordinarily skilled in the art.

### Intermediates

### Intermediate-1:(2E)-3-(2-hydroxyphenyl)prop-2-enal

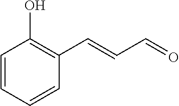 [SMILES: O=C/C=C/c1ccccc1O]

2-(Triphenylphosphoranylidene) acetaldehyde (65.41 g) was added to the stirred solution of salicylaldehyde (25 g) in THF (250 ml) at room temperature under nitrogen atmosphere. The reaction mixture was stirred for 18 h at the same temperature. The solvent was evaporated under reduced pressure to get the desired product. This product was purified by silica gel (60-120 mesh) column chromatography using ethyl acetate and n-hexane as a mixture of solvents to give 15.1 g of (2E)-3-(2-hydroxyphenyl)prop-2-enal as a solid. ¹H NMR (400 MHz, DMSO-d₆) δ 6.82-6.94 (m, 2H), 6.95 (d, J=7.6 Hz, 1H), 7.28-7.35 (m, 1H), 7.64 (dd, J=2.0, 6.4 Hz, 1H), 7.88 (d, J=16.0 Hz, 1H), 9.64 (d, J=8.0 Hz, 1H), 10.43 (brs, 1H).

### Intermediate-2: (2E)-3-(4-fluoro-2-hydroxyphenyl)prop-2-enal

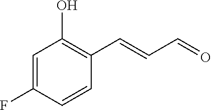 [SMILES: O=C/C=C/c1ccc(F)cc1O]

2-(Triphenylphosphoranylidene) acetaldehyde (7.11 g) was added to the stirred solution of 4-fluoro-2-hydroxybenzaldehyde (3 g) in THF (60 ml) at room temperature under nitrogen atmosphere. The reaction mixture was stirred for 18 h at the same temperature. The solvent was evaporated under reduced pressure to get the desired product. This product was purified by silica gel (60-120 mesh) column chromatography using ethyl acetate and n-hexane as a mixture of solvents to give 2.21 g of (2E)-3-(4-fluoro-2-hydroxyphenyl)prop-2-enal as a solid. ¹H NMR (400 MHz, CDCl₃) δ 6.16 (s, 1H), 6.59 (dd, J=2.4, 8.4 Hz, 1H), 6.69-6.74 (m, 1H), 6.82 (dd, J=8.0, 8.4 Hz, 1H), 7.50 (dd, J=2.4, 6.4 Hz, 1H), 7.72 (d, J=16.4 Hz, 1H), 9.67 (d, J=7.6 Hz, 1H).

### Intermediate-3: (2E)-3-(2-fluoro-6-hydroxyphenyl)prop-2-enal

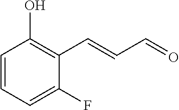 [SMILES: O=C/C=C/c1c(O)cccc1F]

2-(Triphenylphosphoranylidene) acetaldehyde (9.55 g) was added to the stirred solution of 6-fluoro-2-hydroxybenzaldehyde (4 g) in THF (60 ml) at room temperature under nitrogen atmosphere. The reaction mixture was stirred for 18 h at same temperature. The solvent was evaporated under reduced pressure to get the desired product. This product was purified by silica gel (60-120 mesh) column chromatography using ethyl acetate and n-hexane as a mixture of solvents to give 4.10 g of (2E)-3-(2-fluoro-6-hydroxyphenyl)prop-2-enal as a solid. ¹H NMR (400 MHz, CDCl₃) δ 6.68-6.74 (m, 2H), 7.22-7.28 (m, 1H), 7.41 (dd, J=8.8, 8.8 Hz, 1H), 7.62 (s, 1H), 7.78 (d, J=16.0 Hz, 1H), 9.66 (d, 8.0 Hz, 1H).

### Intermediate-4: (2E)-3-(4-chloro-2-hydroxyphenyl)prop-2-enal

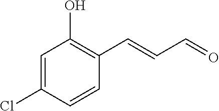 [SMILES: O=C/C=C/c1ccc(Cl)cc1O]

2-(Triphenylphosphoranylidene) acetaldehyde (8.55 g) was added to the stirred solution of 4-chloro-2-hydroxybenzaldehyde (4.0 g) in THF (80 ml) at room temperature under nitrogen atmosphere. The reaction mixture was stirred for 18 h at same temperature. The solvent was evaporated under reduced pressure to get the desired product. This product was purified by silica gel (60-120 mesh) column chromatography using ethyl acetate and n-hexane as a mixture of solvents to give 3.50 g of (2E)-3-(4-chloro-2-hydroxyphenyl)prop-2-enal as a solid. ¹H NMR (400 MHz, CDCl₃) δ 6.20 (s, 1H), 6.87-6.89 (m, 2H), 6.97 (d, J=8.4 Hz, 1H), 7.44 (d, J=8.4 Hz, 1H), 7.70 (d, J=16.0 Hz, 1H), 9.68 (d, J=7.6 Hz, 1H).

### Intermediate-5: (2E)-3-(2-hydroxy-5-methoxyphenyl)prop-2-enal

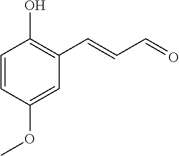

2-(Triphenylphosphoranylidene) acetaldehyde (8.80 g) was added to the stirred solution of 2-hydroxy-5-methoxybenzaldehyde (4.0 g) in THF (80 ml) at room temperature under nitrogen atmosphere. The reaction mixture was stirred for 18 h at same temperature. The solvent was evaporated under reduced pressure to get the desired product. This product was purified by silica gel (60-120 mesh) column chromatography using ethyl acetate and n-hexane as a mixture of solvents to give 8.50 g of (2E)-3-(2-hydroxy-5-methoxyphenyl)prop-2-enal as a solid. ¹H NMR (400 MHz, CDCl₃) δ 3.79 (s, 3H), 5.99 (s, 1H), 6.81 (d, J=8.8 Hz, 1H), 6.86-6.92 (m, 2H), 7.01 (d, J=2.1 Hz, 1H), 7.79 (d, J=16.0 Hz, 1H), 9.68 (d, J=8.0 Hz, 1H).

### Intermediate-6: 3-methoxy-4-[(1E)-3-oxoprop-1-en-1-yl]phenyl acetate

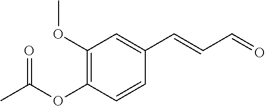 [SMILES: COc1cc(/C=C/C=O)ccc1OC(C)=O]

2-(Triphenylphosphoranylidene) acetaldehyde (3.43 g) was added to the stirred solution of acetylvanillin (3.4.0 g) in acetonitrile (30 ml) at room temperature under nitrogen atmosphere. The reaction mixture was stirred for 18 h at same temperature. The solvent was evaporated under reduced pressure to get the desired product. This product was purified by silica gel (60-120 mesh) column chromatography using ethyl acetate and n-hexane as a mixture of solvents to give 1.95 g of 2-methoxy-4-[(1E)-3-oxoprop-1-en-1-yl]phenyl acetate as a solid. ¹H NMR (400 MHz, DMSO-d₆) δ 2.27 (s, 3H), 3.84 (s, 3H), 6.93 (dd, J=7.6, 8.4 Hz, 1H), 7.19 (d, J=8.0 Hz, 1H), 7.33 (dd, J=1.6, 6.8 Hz, 1H), 7.54 (d, J=2.0 Hz, 1H), 7.72 (d, J=16.0 Hz, 1H), 9.68 (d, J=8.0 Hz, 1H).

### Intermediate-7: (2E)-3-(4-hydroxy-3-methoxyphenyl)prop-2-enal

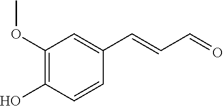

Sodium hydroxide (0.9 g in 10 ml in water) solution was added to the stirred solution of 2-methoxy-4-[(1E)-3-oxoprop-1-en-1-yl]phenyl acetate (Intermediate-6) (2.0 g) in methanol (10 ml) at the room temperature. The reaction mixture was stirred for 2 h at room temperature. The reaction was neutralized with dil. HCl solution and product was extracted with ethyl acetate (50 ml). The organic layer was washed with water (2×100 ml), brine solution (50 ml) and dried over anhydrous Na₂SO₄. The solvent was evaporated under reduced pressure to get 1.86 g of (2E)-3-(4-hydroxy-3-methoxyphenyl)prop-2-enal as a solid. ¹H NMR (400 MHz, CDCl₃) δ 3.94 (s, 3H), 5.60 (brs, 1H), 6.60 (dd, J=7.6, 8.0 Hz, 1H), 6.96 (d, J=7.6 Hz, 1H), 7.07 (s, 1H), 7.12 (d, 8.0 Hz, 1H), 7.40 (d, J=15.6 Hz, 1H), 9.64 (d, J=7.6 Hz, 1H).

### Intermediate-8: 4-[(1E)-3-oxoprop-1-en-1-yl]phenyl acetate

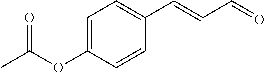

2-(Triphenylphosphoranylidene) acetaldehyde (26.53 g) was added to the stirred solution of 4-formylphenyl acetate (13.0 g) in acetonitrile (100 ml) at room temperature under nitrogen atmosphere. The reaction mixture was stirred for 16 h at same temperature. The solvent was evaporated under reduced pressure to get the product. This product was purified by silica gel (60-120 mesh) column chromatography using ethyl acetate and n-hexane as a mixture of solvents to give 6.50 g of 4-[(1E)-3-oxoprop-1-en-1-yl]phenyl acetate as a solid. ¹H NMR (400 MHz, CDCl₃) δ 2.29 (s, 3H), 6.85 (dd, J=7.6, 8.0 Hz, 1H), 7.24 (d, J=8.8 Hz, 2H), 7.75 (d, J=16.0 Hz, 1H), 7.81 (d, J=8.4 Hz, 2H), 9.67 (d, J=7.6 Hz, 1H).

### Intermediate-9: (2E)-2-benzylidenebutanal

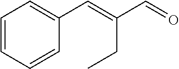

The solution of butyraldehyde (13.20 g) in ethanol (75 ml) was added in dropwise to the stirred solution of benzaldehyde (31.2 g) and aq·KOH (45%, 5 ml) and ethanol (75 ml) at 10° C. The reaction mixture was stirred at RT for 3 days. The solvent was evaporated under the reduced pressure, then the residue was dissolved in ethyl acetate (200 ml). The mixture was washed with water (2×200 ml) followed by brine solution (100 ml) and dried over anhydrous sodium sulfate. The mixture was concentrated under the reduced pressure to afford the desired product. This product was purified by silica gel column chromatography using ethyl acetate and n-hexane as a mixture of solvents to give 10.31 g of (2E)-2-benzylidenebutanal as a viscose liquid. ¹H NMR (400 MHz, CDCl₃) δ 1.14 (t, J=7.6 Hz, 3H), 2.56 (q, J=7.6 Hz, 2H), 7.21 (s, 1H), 7.30-7.52 (m, 5H), 9.55 (s, 1H).

### Intermediate-10: (2E)-3-(furan-2-yl)-2-methylprop-2-enal

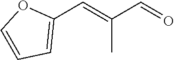

The solution of propionaldehyde (17.35 g) in ethanol (100 ml) was added in dropwise fashion to the stirred solution of furfural (46.32 g) and aq·KOH (45%, 12 ml) and ethanol (100 ml) at 10° C. The reaction mixture was stirred at RT for 3 days. The solvent was evaporated under the reduced pressure, and the mixture was dissolved in ethyl acetate (200 ml). The mixture was washed with (2×200 ml) water followed by brine solution (100 ml) and dried over anhydrous sodium sulfate. The mixture was concentrated under the reduced pressure to afford the desired product. This product was purified by silica gel (60-120 mesh) column chromatography using ethyl acetate and n-hexane as a mixture of solvents to give 8.52 g of (2E)-3-(furan-2-yl)-2-methylprop-2-enal as a viscose liquid. ¹H NMR (400 MHz, CDCl₃) δ 2.11 (s, 3H), 6.56-6.57 (m, 1H), 6.78 (d, J=2.8 Hz, 1H), 7.03 (s, 1H), 7.62 (s, 1H), 9.50 (s, 1H).

### Intermediate-11: (2E)-2-[(furan-2-yl)methylidene]butanal

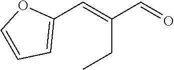

The solution of butyraldehyde (4.50 g) in ethanol (50 ml) was added in dropwise to the stirred solution of benzaldehyde (10 g) and aq·KOH (45%, 3 ml) and ethanol (50 ml) at 10° C. The reaction mixture was stirred at room temperature for 3 days. The solvent was evaporated under the reduced pressure and the mixture was dissolved in ethyl acetate (100 ml). The mixture was washed with water (2×100 ml) followed by brine solution (100 ml) and dried over anhydrous sodium sulfate. The mixture was concentrated under the reduced pressure to afford the desired product. This product was purified by silica gel (60-120 mesh) column chromatography using ethyl acetate and n-hexane as a mixture of solvents to give 3.51 g of (2E)-2-[(furan-2-yl)methylidene]butanal as a viscose liquid. ¹H NMR (400 MHz, CDCl₃) δ 1.08 (t, J=7.6 Hz, 3H), 2.65 (q, J=7.6 Hz, 2H), 6.55-6.57 (m, 1H), 6.77 (d, J=3.2 Hz, 1H), 6.93 (s, 1H), 7.62 (d, J=1.2 Hz, 1H), 9.46 (s, 1H).

### Intermediate-12: 2-[(1E)-3-oxoprop-1-en-1-yl]phenyl benzoate

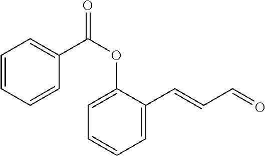 [SMILES: O=C/C=C/c1ccccc1OC(=O)c1ccccc1]

Benzoyl chloride (5 g) was added to the solution of (2E)-3-(2-hydroxyphenyl)prop-2-enal (6 g) and triethyl amine (8.28 g) in dichloromethane (50 ml) at RT under nitrogen atmosphere. The reaction mixture was stirred for 1 h at the same temperature. The reaction mixture was washed with (2×50 ml) water followed by brine solution (100 ml) and dried over anhydrous sodium sulfate. The mixture was concentrated under the reduced pressure to afford 8 g of 2-[(1E)-3-oxoprop-1-en-1-yl]phenyl benzoate as solid. ¹H NMR (400 MHz, CDCl₃) δ 6.88 (dd, J=7.6 Hz, 8.0 Hz, 1H), 7.41-7.45 (m, 2H), 7.57-7.65 (m, 3H), 7.76-7.81 (m, 2H), 8.00 (d, J=8.0 Hz, 1H), 8.21 (d, J=7.2 Hz, 2H), 9.64 (d, J=8.0 Hz, 1H). ESI-MS: m/z 253.20 (M+H)⁺.

### Intermediate-13: 4-[(1E)-3-oxoprop-1-en-1-yl]benzonitrile

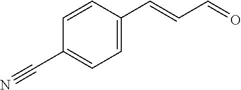 [SMILES: N#Cc1ccc(/C=C/C=O)cc1]

2-(Triphenylphosphoranylidene) acetaldehyde (7 g) was added to the stirred solution of 4-formylbenzonitrile (3 g) in acetonitrile (100 ml) at room temperature under nitrogen atmosphere. The reaction mixture was stirred for 16 h at same temperature. The solvent was evaporated under reduced pressure to get the product. This product was purified by silica gel (60-120 mesh) column chromatography using ethyl acetate and n-hexane as a mixture of solvents to give 2.91 g of 4-[(1E)-3-oxoprop-1-en-1-yl]benzonitrile. ¹H NMR (400 MHz, CDCl₃) δ 6.77 (dd, J=7.6 Hz, 8.0 Hz, 1H), 7.48 (d, J=16.0 Hz, 1H), 7.67 (d, J=8.0 Hz, 2H), 7.73 (d, J=8.0 Hz, 2H), 9.76 (d, J=7.6 Hz, 1H). ESI-MS: m/z 158.10 (M+H)⁺.

### Intermediate-14: (2E)-3-(thiophen-3-yl)prop-2-enal

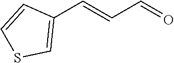

2-(Triphenylphosphoranylidene) acetaldehyde (8.2 g) was added to the stirred solution of thiophene-3-carbaldehyde (3 g) in acetonitrile (100 ml) at room temperature under nitrogen atmosphere. The reaction mixture was stirred for 24 h at same temperature. The solvent was evaporated under reduced pressure to get the product. This product was purified by silica gel (60-120 mesh) column chromatography using ethyl acetate and n-hexane as a mixture of solvents to give 2.46 g of (2E)-3-(thiophen-3-yl)prop-2-enal. ¹H NMR (400 MHz, CDCl₃) δ 6.69 (dd, J=7.6 Hz, 8.0 Hz, 1H), 7.48-7.74 (m, 3H), 8.09 (d, J=2.0 Hz, 1H), 9.62 (d, J=8.0 Hz, 1H). ESI-MS: m/z 139.20 (M+H)⁺.

### Intermediate-15: (2E)-3-(1,3-benzothiazol-2-yl)prop-2-enal

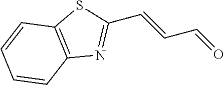 [SMILES: O=C/C=C/c1nc2ccccc2s1]

2-(Triphenylphosphoranylidene) acetaldehyde (5.6 g) was added to the stirred solution of 1,3-benzothiazole-2-carbaldehyde (3 g) in acetonitrile (80 ml) at room temperature under nitrogen atmosphere. The reaction mixture was stirred for 24 h at the same temperature. The solvent was evaporated under the reduced pressure to get the product. This product was purified by silica gel column chromatography using mixture of ethyl acetate and n-hexane to give 2.68 g of (2E)-3-(1,3-benzothiazol-2-yl)prop-2-enal. ¹H NMR (400 MHz, CDCl₃) δ 7.02 (dd, J=7.6 Hz, 8.0 Hz, 1H), 7.54-7.63 (m, 2H), 8.08 (d, J=16.0 Hz, 1H), 8.12 (d, J=8.0 Hz, 1H), 8.21 (d, J=7.6 Hz, 1H), 9.83 (d, J=7.6 Hz, 1H). ESI-MS: m/z 190.30 (M+H)⁺.

### Intermediate-16: (2E)-3-(1,3-thiazol-2-yl)prop-2-enal

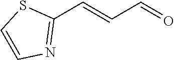

2-(Triphenylphosphoranylidene) acetaldehyde (8.1 g) was added to the stirred solution of 1,3-thiazole-2-carbaldehyde (3 g) in acetonitrile (100 ml) at room temperature under nitrogen atmosphere. The reaction mixture was stirred for 24 h at the same temperature. The solvent was evaporated under the reduced pressure to get the product. This product was purified by silica gel column chromatography using mixture of ethyl acetate and n-hexane to give 2.06 g of (2E)-3-(1,3-thiazol-2-yl)prop-2-enal. ¹H NMR (400 MHz, CDCl₃) δ 6.87 (dd, J=7.6 Hz, 8.0 Hz, 1H), 7.98 (d, J=16.0 Hz, 1H), 8.06 (d, J=3.2 Hz, 1H), 8.10 (d, J=3.2 Hz, 1H), 9.74 (d, J=7.6 Hz, 1H). ESI-MS: m/z 140.10 (M+H)⁺.

### EXAMPLES

### Example-1: Preparation of (E)-N²-[(2E)-3-(2-hydroxyphenyl)prop-2-en-1-ylidene]-L-arginine

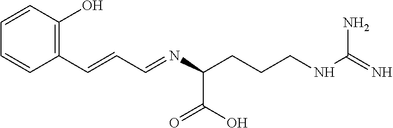 [SMILES: N=C(N)NCCC[C@H](/N=C/C=C/c1ccccc1O)C(=O)O]

L-arginine (1.15 g) was added to the solution of 2-hydroxy-cinnamaldehyde (1 g) in ethanol (25 ml) and the reaction mixture was stirred at RT for 12 h, The solid was filtered, washed with cold ethanol (20 ml) and dried under high vacuum to give 1.58 g of (E)-N²-[(2E)-3-(2-hydroxyphenyl)prop-2-en-1-ylidene]-L-arginine as a solid. ¹H NMR (400 MHz, DMSO-d₆) δ 1.39-1.45 (m, 2H), 1.60-1.82 (m, 2H), 3.2-3.18 (m, 2H), 3.54 (t, J=6.4 Hz, 1H), 6.72 (t, J=7.2 Hz, 1H), 6.85 (d, J=8.0 Hz, 1H), 6.87-6.94 (m, 1H), 7.08 (t, J=8.0 Hz, 1H), 7.19 (d, J=8.8 Hz, 1H), 7.40 (d, J=7.2 Hz, 1H), 7.59-8.01 (brs, 4H), 7.92 (d, J=8.8 Hz, 1H), 9.11 (s, 1H), 10.25 (brs, 1H); ESI-MS: m/z 305.10 (M+H)⁺.

### Example-2: Preparation of (E)-N²-[(2E)-3-(4-fluoro-2-hydroxyphenyl)prop-2-en-1-ylidene]-L-arginine

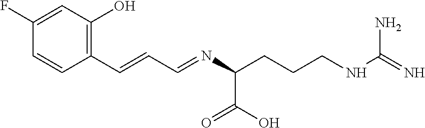 [SMILES: N=C(N)NCCC[C@H](/N=C/C=C/c1ccc(F)cc1O)C(=O)O]

L-arginine (1 g) was added to the solution of (2E)-3-(4-fluoro-2-hydroxyphenyl)prop-2-enal (1 g) in ethanol (20 ml) and the reaction mass stirred at RT for 16 h, The solid was filtered, washed with cold ethanol (10 ml) and n-hexane (25 ml). The solid was dried under high vacuum to give 1.51 g of (E)-N²-[(2E)-3-(4-fluoro-2-hydroxyphenyl)prop-2-en-1-ylidene]-L-arginine as a solid. ¹H NMR (400 MHz, DMSO-d₆) δ 1.41-1.90 (m, 4H), 3.01-3.19 (m, 2H), 3.58 (t, J=6.4 Hz, 1H), 6.52 (t, J=7.6 Hz, 1H), 6.63 (d, J=11.2 Hz, 1H), 6.96-7.02 (m, 1H), 7.15 (d, J=15.6 Hz, 1H), 7.43 (t, J=8.8 Hz, 1H), 7.70 (brs, 5H), 7.93 (d, J=8.8 Hz, 1H), 9.01 (brs, 1H); ESI-MS: m/z 323.10 (M+H)⁺.

### Example-3: Preparation of (E)-N²-[(2E)-3-(6-fluoro-2-hydroxyphenyl)prop-2-en-1-ylidene]-L-arginine

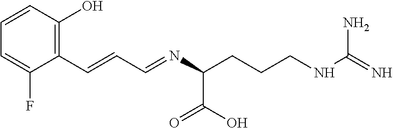 [SMILES: N=C(N)NCCC[C@H](/N=C/C=C/c1c(O)cccc1F)C(=O)O]

L-arginine (1.0 g) was added to the solution of (2E)-3-(6-fluoro-2-hydroxyphenyl)prop-2-enal (1 g) in ethanol (20 ml) and the reaction mixture was stirred at RT for 16 h, The solid was filtered, washed with cold ethanol (10 ml) and n-hexane (25 ml) and it was dried under high vacuum to give 1.53 g of (E)-N²-[(2E)-3-(6-fluoro-2-hydroxyphenyl)prop-2-en-1-ylidene]-L-arginine as a solid. ¹H NMR (400 MHz, DMSO-d₆) δ 1.41-1.60 (m, 2H), 1.65-1.90 (m, 2H), 3.00-3.20 (m, 2H), 3.60 (t, J=6.4 Hz, 1H), 6.55 (t, J=8.8 Hz, 1H), 6.74 (d, J=8.4 Hz, 1H), 7.06-7.28 (m, 3H), 7.76 (brs, 4H), 7.96 (d, J=8.4 Hz, 1H), 8.95 (brs, 1H), 11.95 (brs, 1H); ESI-MS: m/z 323.10 (M+H)⁺.

### Example-4: Preparation of (E)-N²-[(2E)-3-(4-chloro-2-hydroxyphenyl)prop-2-en-1-ylidene]-L-arginine

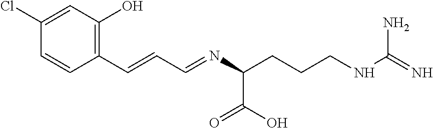 [SMILES: N=C(N)NCCC[C@H](/N=C/C=C/c1ccc(Cl)cc1O)C(=O)O]

L-arginine (0.92 g) was added to the solution of (2E)-3-(4-chloro-2-hydroxyphenyl)prop-2-enal (1 g) in ethanol (20 ml) and the reaction mixture was stirred at RT for 16 h, The solid was filtered, washed with cold ethanol (10 ml) and n-hexane (20 ml) and dried under high vacuum to give 1.57 g of (E)-N²-[(2E)-3-(4-chloro-2-hydroxyphenyl)prop-2-en-1-ylidene]-L-arginine as a solid. ¹H NMR (400 MHz, DMSO-d₆) δ 1.40-1.50 (m, 2H), 1.65-1.84 (m, 2H), 3.00-3.17 (m, 2H), 3.57 (t, J=6.4 Hz, 1H), 6.74 (d, J=8.8 Hz, 1H), 6.89 (s, 1H), 7.09-7.16 (m, 2H), 7.42 (d, J=8.4 Hz, 1H), 7.75 (brs, 5H), 7.94 (d, J=8.4 Hz, 1H), 9.12 (brs, 1H); ESI-MS: m/z 338.82 (M+H)⁺.

### Example-5: Preparation of (E)-N²-[(2E)-3-(2-hydroxy-5-methoxyphenyl)prop-2-en-1-ylidene]-L-arginine

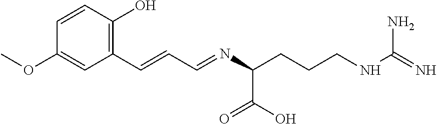 [SMILES: COc1ccc(O)c(/C=C/C=N/[C@@H](CCCNC(=N)N)C(=O)O)c1]

L-arginine (0.94 g) was added to the solution of (2E)-3-(5-methoxy-2-hydroxyphenyl)prop-2-enal (1 g) in ethanol (20 ml) and the reaction mixture was stirred at RT for 16 h, The solid was filtered, washed with cold ethanol (10 ml) and n-hexane (20 ml) and dried under high vacuum to give 1.57 g of (E)-N²-[(2E)-3-(2-hydroxy-5-methoxyphenyl)prop-2-en-1-ylidene]-L-arginine as a solid. ¹H NMR (400 MHz, DMSO-d₆) δ 1.40-1.48 (m, 2H), 1.60-1.85 (m, 2H), 3.00-3.14 (m, 2H), 3.56 (t, J=6.4 Hz, 1H), 3.69 (s, 3H), 3.68-6.93 (m, 3H), 7.04 (d, J=2.8 Hz, 1H), 7.18 (d, J=8.8 Hz, 1H), 7.35 (brs, 5H), 7.95 (d, J=9.2 Hz, 1H), 9.58 (brs, 1H); ESI-MS: m/z 335.10 (M+H)⁺.

### Example-6: Preparation of (E)-N²-[(2E)-3-(2-methoxyphenyl)prop-2-en-1-ylidene]-L-arginine

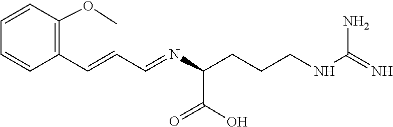 [SMILES: COc1ccccc1/C=C/C=N/[C@@H](CCCNC(=N)N)C(=O)O]

The solution of 2-methoxy-cinnamaldehyde (5.12 g) in ethanol (20 ml) was added to the slurry of L-arginine (5 g) in ethanol (50 ml) and the reaction mixture was stirred at RT for 16 h. The solid was filtered, washed with ethanol (25 ml) and dried under high vacuum to give 7.96 g of (E)-N²-[(2E)-3-(2-methoxyphenyl)prop-2-en-1-ylidene]-L-arginine as a solid. ¹H NMR (400 MHz, DMSO-d₆) δ 1.40-1.51 (m 2H), 1.66-1.81 (m 2H), 3.01-3.15 (m, 2H), 3.58 (t, J=6.4 Hz, 1H), 383 (s, 3H) 6.86-6.94 (m, 2H), 7.03 (d, J=8.4 Hz, 1H), 7.27 (d, J=17.6 Hz, 1H), 7.33 (t, J=8.4 Hz, 1H), 7.56 (d, J=6.8 Hz, 1H), 7.40-7.80 (brs, 4H), 7.97 (d, J=9.2 Hz, 1H), 9.39 (s, 1H); ESI-MS: m/z 318.30 (M⁺).

### Example-7: Preparation of (E)-N²-[(2E)-3-(4-methoxyphenyl)prop-2-en-1-ylidene]-L-arginine

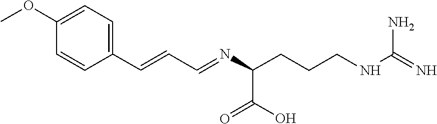 [SMILES: COc1ccc(/C=C/C=N/[C@@H](CCCNC(=N)N)C(=O)O)cc1]

4-methoxy-cinnamaldehyde (5.12 g) was slowly added to the slurry of L-arginine (5 g) in ethanol (50 ml) and the reaction mixture was stirred at RT for 16 h. The solid was filtered, washed with ethanol (25 ml) and dried under high vacuum to give 7.81 g of (E)-N²-[(2E)-3-(4-methoxyphenyl)prop-2-en-1-ylidene]-L-arginine as a solid. ¹H NMR (400 MHz, DMSO-d₆) δ 1.45-1.80 (m, 4H), 3.06-3.10 (m, 2H), 3.52 (t, J=6.4 Hz, 1H), 3.82 (s, 3H), 6.72-6.78 (m, 1H), 6.94-7.00 (m, 3H), 7.05-7.45 (brs, 4H), 7.52 (d, J=8.4 Hz, 2H), 7.94 (d, J=9.20 Hz, 1H), 9.65 (s, 1H); ESI-MS: m/z 318.40 (M⁺).

### Example-8: Preparation of (E)-N²-{(2E)-3-[4-(acetyloxy)-3-methoxyphenyl]prop-2-en-1-ylidene}-L-arginine

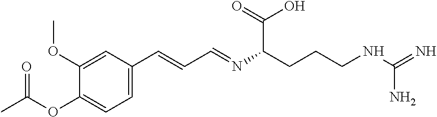 [SMILES: COc1cc(/C=C/C=N/[C@@H](CCCNC(=N)N)C(=O)O)ccc1OC(C)=O]

L-arginine (0.75 g) was added to the solution of 2-methoxy-4-[(1E)-3-oxoprop-1-en-1-yl]phenyl acetate (1 g) in ethanol (20 ml) and the reaction mixture was stirred at RT for 16 h under nitrogen atmosphere. The solid product was filtered, washed with cold ethanol (20 ml) followed by n-hexane (20 ml) then dried under high vacuum to give 0.92 g (E)-N²-{(2E)-3-[4-(acetyloxy)-3-methoxyphenyl]prop-2-en-1-ylidene}-L-arginine as a solid. ¹H NMR (400 MHz, DMSO-d₆) δ 1.42-1.55 (m, 2H), 1.63-1.80 (m, 2H), 2.52 (s, 3H), 3.00-3.15 (m, 2H), 3.58 (t, J=6.6 Hz, 1H), 3.80 (s, 3H), 6.90-7.15 (m, 4H), 7.34 (s, 1H), 7.54 (brs, 4H), 7.92 (d, J=8.4 Hz, 1H), 9.44 (s, 1H); ESI-MS: m/z 377.30 (M+H)⁺.

### Example-9: Preparation of (E)-N²-[(2E)-3-phenylprop-2-en-1-ylidene]-L-arginine

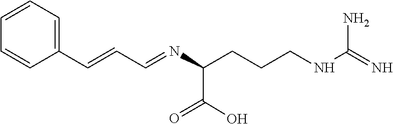 [SMILES: N=C(N)NCCC[C@H](/N=C/C=C/c1ccccc1)C(=O)O]

Cinnamaldehyde (7.44 g) was added to the stirred solution of L-arginine (10 g) in water (170 ml) and the reaction mixture was stirred at RT for 2 h. The obtained solid was filtered, washed with water (100 ml) and dried under high vacuum to get 15.80 g of (E)-N²-[(2E)-3-phenylprop-2-en-1-ylidene]-L-arginine as a solid. ¹H NMR (400 MHz, DMSO-d₆) δ 1.46-1.52 (m, 2H), 1.62-1.67 (m, 1H), 1.80-1.83 (m, 1H), 3.08-3.11 (m, 2H), 3.57 (t, J=6.4 Hz, 1H), 6.88-6.91 (m, 1H), 7.05 (d, J=16.0 Hz, 1H), 7.30-7.40 (m, 3H), 7.57 (d, J=7.6 Hz, 1H), 7.21-8.10 (brs, 4H), 8.00 (d, J=8.8 Hz, 2H), 9.56 (br s, 1H); ESI-MS: m/z 288.85 (M)⁺.

### Example-10: Preparation of (E)-N²-[(2E)-2-methyl-3-phenylprop-2-en-1-ylidene]-L-arginine

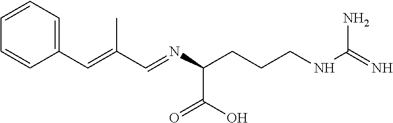 [SMILES: CC(/C=N/[C@@H](CCCNC(=N)N)C(=O)O)=C\c1ccccc1]

L-arginine (2.28 g) was added to the solution of α-methyl cinnamaldehyde (2 g) in ethanol (40 ml) and the reaction mixture was stirred at RT for 16 h under nitrogen atmosphere. The solid was filtered, washed with cold ethanol (20 ml) followed by n-hexane (20 ml) and then dried under high vacuum to give 3.73 g of (E)-N²-[(2E)-2-methyl-3-phenylprop-2-en-1-ylidene]-L-arginine as a solid. ¹H NMR (400 MHz, DMSO-d₆) δ 1.40-1.49 (m, 2H), 1.59-1.66 (m, 1H), 1.83-1.88 (m, 1H), 3.09-3.15 (m, 2H), 3.33 (s, 3H), 3.65 (t, J=6.4 Hz, 1H), 6.86 (s, 1H), 7.28-7.45 (m, 5H), 7.55 (brs, 4H), 7.97 (s, 1H), 9.50 (brs, 1H); ESI-MS: m/z 303.10 (M+H)⁺.

### Example-11: Preparation of (E)-N²-[(2E)-2-benzylidenebutylidene]-L-arginine

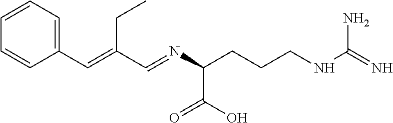 [SMILES: CCC(/C=N/[C@@H](CCCNC(=N)N)C(=O)O)=C\c1ccccc1]

(2E)-2-benzylidenebutanal (1 g) was added to the stirred slurry of L-arginine (1.03 g) in ethanol (30 ml) and the reaction mixture was stirred at RT for 5 h under nitrogen atmosphere. The solid was filtered, washed with cold ethanol and dried under high vacuum to give 1.09 g of (E)-N²-[(2E)-2-benzylidenebutylidene]-L-arginine as a solid. ¹H NMR (400 MHz, DMSO-d₆) δ 1.09 (t, J=7.2 Hz, 3H), 1.45-1.46 (m, 2H), 1.62-1.67 (m, 1H), 1.85-1.88 (m, 1H), 2.53 (q, J=7.6 Hz, 2H), 3.08-3.15 (m, 2H), 3.60 (t, J=6.4 Hz, 1H), 6.76 (s, 1H), 7.31-7.38 (m, 2H), 7.41-7.59 (m, 3H), 7.59 (brs. 2H), 7.86 (s, 1H), 7.96 (brs, 2H), 9.54 (brs, 1H); ESI-MS: m/z 317.40 (M+H)⁺.

### Example-12: Preparation of (E)-N²-[(2E)-2-benzylideneheptylidene]-L-arginine

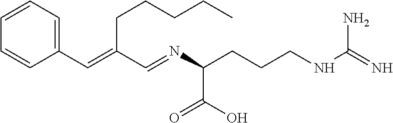 [SMILES: CCCCCC(/C=N/[C@@H](CCCNC(=N)N)C(=O)O)=C\c1ccccc1]

α-amyl cinnamaldehyde (6.77 g) was added to the stirred slurry of L-arginine (5.0 g) in methanol (50 ml) and the reaction mixture was stirred at RT for 6 h under nitrogen atmosphere. The solvent was removed under vacuum. The residue was purified by using acetone (20 ml) and n-hexane (60 ml) to give 8.91 g of ((E)-N²-[(2E)-2-benzylideneheptylidene]-L-arginine as a solid. ¹H NMR (400 MHz, DMSO-d₆) δ 0.80-0.87 (m, 3H), 1.20-1.31 (m, 4H), 1.46-1.55 (m, 4H), 1.63-1.68 (m, 1H), 1.82-1.87 (m, 1H), 2.46-2.56 (m, 2H), 3.10-3.21 (m 2H), 3.61 (t, J=6.4, 1H), 6.79 (s, 1H), 7.27-7.53 (m, 5H), 7.55-8.00 (brs, 4H), 7.87 (s, 1H), 9.32 (brs, 1H); ESI-MS: m/z 359.40 (M+H)⁺.

### Example-13: Preparation of (E)-N²-[(2E)-2-benzylideneoctylidene]-L-arginine

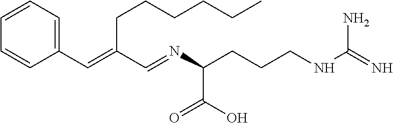 [SMILES: CCCCCCC(/C=N/[C@@H](CCCNC(=N)N)C(=O)O)=C\c1ccccc1]

α-Hexyl cinnamaldehyde (6.52 g) was added to the stirred slurry of L-arginine (5.0 g) in methanol (50 ml) and the reaction mixture was stirred at RT for 6 h under nitrogen atmosphere. The solvent was removed under vacuum. The residue was purified by using acetone (25 ml) and n-hexane (50 ml) to give 10.5 g of (E)-N²-[(2E)-2-benzylideneoctylidene]-L-arginine as a solid. ¹H NMR (400 MHz, DMSO-d₆) δ 0.79-0.85 (m, 3H), 1.20-1.28 (m, 6H), 1.45-1.55 (m, 4H), 1.63-1.87 (m, 2H), 2.44-2.56 (m, 2H), 3.10-3.20 (m, 2H), 3.16 (t, J=6.4 Hz, 1H), 6.79 (s, 1H), 7.32-7.56 (m, 5H), 7.50-8.10 (brs, 4H), 7.87 (s, 1H), 9.54 (brs, 1H); ESI-MS: m/z 373.30 (M+H)⁺.

### Example-14: Preparation of (E)-N²-[(2E)-3-(furan-2-yl)prop-2-en-1-ylidene]-L-arginine

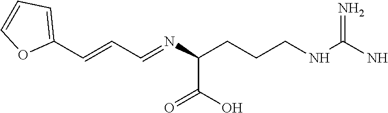 [SMILES: N=C(N)NCCC[C@H](/N=C/C=C/c1ccco1)C(=O)O]

(2E)-3-(Furan-2-yl)prop-2-enal (1 g) was added to the stirred slurry of L-arginine (1.35 g) in ethanol (25 ml) and the reaction mixture was stirred at RT for 7 h under nitrogen atmosphere. The reaction mixture was filtered, washed with cold ethanol followed by n-hexane and dried under high vacuum to give 1.69 g of ((E)-N²-[(2E)-3-(furan-2-yl)prop-2-en-1-ylidene]-L-arginine as solid. ¹H NMR (400 MHz, DMSO-d₆) δ 1.40-1.49 (m, 2H), 1.62-1.67 (m, 1H), 1.72-1.79 (m, 1H), 3.05-3.14 (m, 2H), 3.56 (t, J=6.4 Hz, 1H), 6.55-6.61 (m, 2H), 6.71 (d, J=3.2 Hz, 1H), 6.93 (d, J=9.2 Hz, 1H), 7.51-8.21 (brs, 4H), 7.74 (d, J=1.2 Hz, 1H), 7.93 (d, J=9.2 Hz, 1H), 9.35 (brs, 1H); ESI-MS: m/z 297.10 (M+H)⁺.

### Example-15: Preparation of (E)-N²-[(2E)-3-(furan-2-yl)-2-methylprop-2-en-1-ylidene]-L-arginine

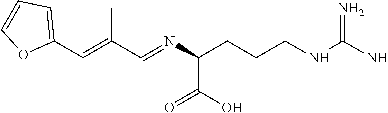 [SMILES: CC(/C=N/[C@@H](CCCNC(=N)N)C(=O)O)=C\c1ccco1]

L-arginine (0.98 g) was added to the solution of (2E)-2-[(furan-2-yl)-2-methylprop-2-enal (0.8 g) ethanol (20 ml) and the reaction mixture was stirred at RT for 6 h under nitrogen atmosphere. The reaction mixture was filtered, washed with cold ethanol followed by n-hexane and dried under high vacuum to give 0.61 g of (E)-N²-[(2E)-3-(furan-2-yl)-2-methylprop-2-en-1-ylidene]-L-arginine as solid. ¹H NMR (400 MHz, DMSO-d₆) δ 1.41-1.50 (m, 2H), 1.57-1.62 (m, 1H), 1.80-1.85 (m, 1H), 2.09 (s, 3H), 3.01-3.09 (m, 2H), 3.61 (t, J=6.4 Hz, 1H), 6.61 (s, 1H), 6.66-6.68 (m, 2H), 7.45 (brs, 4H), 7.77 (s, 1H), 7.90 (s, 1H), 9.50 (brs, 1H); ESI-MS: m/z 293.20 (M+H)⁺.

### Example-16: Preparation of (E)-N²-{(2E)-2-[(furan-2-yl)methylidene] butylidene}-L-arginine

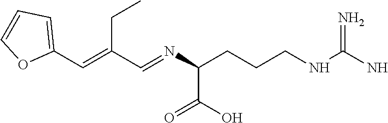 [SMILES: CCC(/C=N/[C@@H](CCCNC(=N)N)C(=O)O)=C\c1ccco1]

(2E)-2-[(furan-2-yl)methylidene]butanal (1 g) was added to the stirred slurry of L-arginine (1.1 g) in ethanol (25 ml) and the reaction mixture was stirred at RT for 7 h under nitrogen atmosphere. The reaction mixture was filtered, washed with cold ethanol followed by n-hexane and dried under high vacuum to give 1.18 g of (E)-N²-{(2E)-2-[(furan-2-yl)methylidene]butylidene}-L-arginine as solid. ¹H NMR (400 MHz, DMSO-d₆) δ 1.01 (t, J=7.6 Hz, 3H), 1.42-1.49 (m, 2H), 1.59-1.63 (m, 1H), 1.83-1.86 (m, 1H), 2.65-2.66 (m, 2H), 2.65-2.70 (m, 2H), 3.08-3.11 (m, 2H), 3.59 (t, J=6.4 Hz, 1H), 6.58 (s, 1H), 6.60 (s, 1H), 6.65 (d, J=3.2 Hz, 1H), 7.80 (br s, 2H), 8.01 (brs, 2H), 9.45 (brs, 1H); ESI-MS: m/z 307.40 (M+H)⁺.

### Example-17: Preparation of (E)-N²-[(2E)-3-(4-hydroxy-3-methoxyphenyl)prop-2-en-1-ylidene]-L-arginine

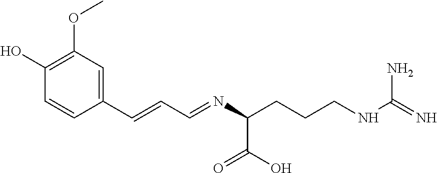 [SMILES: COc1cc(/C=C/C=N/[C@@H](CCCNC(=N)N)C(=O)O)ccc1O]

L-arginine (0.84 g) was added to the solution of (2E)-3-(4-hydroxy-3-methoxyphenyl)prop-2-enal (1 g) in ethanol (10 ml) and the reaction mixture was stirred at RT for 16 h under nitrogen atmosphere. The solvent was evaporated under reduced pressure to get the residue. The residue was purified using acetone (10 ml) and n-hexane (20 ml) to give 0.69 g of (E)-N²-[(2E)-3-(4-hydroxy-3-methoxyphenyl)prop-2-en-1-ylidene]-L-arginine as a solid. ¹H NMR (400 MHz, DMSO-d₆) δ 1.44-1.88 (m, 4H), 3.00-3.09 (m, 2H), 3.65 (t, J=6.0 Hz, 1H), 3.80 (s, 3H), 6.66-6.77 (m, 2H), 6.81-7.14 (m, 2H), 7.30 (s, 1H), 7.59 (brs, 5H), 7.91 (d, J=8.8 Hz, 1H), 9.57 (brs, 1H); ESI-MS: m/z 335.20 (M+H)⁺.

### Example-18: Preparation of (E)-N²-{(2E)-3-[4-(acetyloxy)phenyl]prop-2-en-1-ylidene}-L-arginine

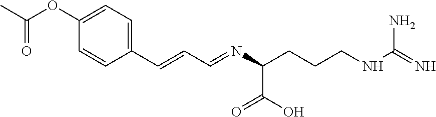 [SMILES: CC(=O)Oc1ccc(/C=C/C=N/[C@@H](CCCNC(=N)N)C(=O)O)cc1]

L-arginine (0.87 g) was added to the solution of 4-[(1E)-3-oxoprop-1-en-1-yl]phenyl acetate (1 g) in ethanol (15 ml) at room temperature. The reaction mixture was stirred for 16 h at RT under nitrogen atmosphere. The solid product was filtered, washed with cold ethanol (10 ml) followed by n-hexane (40 ml) and then dried under high vacuum to give 1.32 g of (E)-N²-{(2E)-3-[4-(acetyloxy)phenyl]prop-2-en-1-ylidene}-L-arginine as a solid. ¹H NMR (400 MHz, DMSO-d₆) δ1.40-1.45 (m, 2H), 1.61-1.85 (m, 2H), 2.27 (s, 3H), 3.00-3.15 (m, 2H), 3.56 (t, J=6.4 Hz, 1H), 6.84-6.90 (m, 1H), 7.05 (d, J=16.0 Hz, 1H), 7.14 (d, J=8.4 Hz, 2H), 7.39 (brs, 4H), 7.62 (d, J=8.4 Hz, 2H), 7.98 (d, J=8.8 Hz, 1H), 9.66 (brs, 1H); ESI-MS: m/z 347.38 (M+H)⁺.

### Example-19: Preparation of (E)-N²-{(2E)-3-[2-(benzoyloxy)phenyl]prop-2-en-1-ylidene}-L-arginine

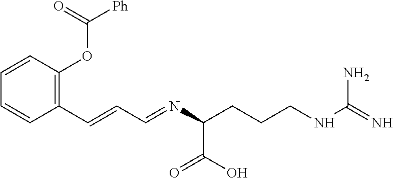 [SMILES: N=C(N)NCCC[C@H](/N=C/C=C/c1ccccc1OC(=O)c1ccccc1)C(=O)O]

L-arginine (0.66 g) was added to the solution of 2-[(1E)-3-oxoprop-1-en-1-yl]phenyl benzoate (1 g) in ethanol (20 ml) at room temperature. The reaction mixture was stirred for 18 h at RT under nitrogen atmosphere. The solid product was filtered, washed with n-hexane (20 ml) and then dried under high vacuum to give 1.07 g of (E)-N²-{(2E)-3-[2-(benzoyloxy)phenyl]prop-2-en-1-ylidene}-L-arginine as a solid. ¹H NMR (400 MHz, DMSO-d₆) δ 1.39 (br s, 2H), 1.56-1.59 (m, 1H), 1.78-1.76 (m, 1H), 3.03 (br s, 2H), 3.50 (t, J=6.2 Hz, 1H), 6.89-6.96 (m, 1H), 7.07 (d, J=16.0 Hz, 1H), 7.20-7.88 (br s, 3H), 7.30-7.37 (m, 3H), 7.42 (t, J=7.4 Hz, 1H), 7.63 (t, J=7.6 Hz, 2H), 7.77 (t, J=7.4 Hz, 1H), 7.87 (d, J=7.2 Hz, 1H), 7.95 (d, J=8.4 Hz, 1H), 8.18 (d, J=7.6 Hz, 2H), 9.50 (br s, 1H); ESI-MS: m/z 409.76 (M+H)⁺.

### Example-20: Preparation of (E)-N²-[(2E)-3-(4-cyanophenyl)prop-2-en-1-ylidene]-L-arginine

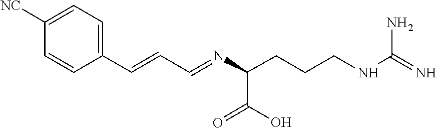 [SMILES: N#Cc1ccc(/C=C/C=N/[C@@H](CCCNC(=N)N)C(=O)O)cc1]

L-arginine (1.1 g) was added to the solution of 4-[(1E)-3-oxoprop-1-en-1-yl]benzonitrile (1 g) in ethanol (20 ml) at room temperature. The reaction mixture was stirred for 18 h at RT under nitrogen atmosphere. The solid product was filtered, washed with n-hexane (20 ml) and then dried under high vacuum to give 1.21 g of (E)-N²-[(2E)-3-(4-cyanophenyl)prop-2-en-1-ylidene]-L-arginine as a solid. ¹H NMR (400 MHz, DMSO-d₆) δ 1.45 (br s, 2H), 1.65-1.67 (m, 1H), 1.84-1.85 (m, 1H), 3.08 (br s, 2H), 3.60 (t, J=6.2 Hz, 1H), 7.01-7.07 (m, 1H), 7.13 (d, J=16.0 Hz, 1H), 7.53 (br s, 4H), 7.77 (d, J=8.4 Hz, 2H), 7.82 (d, J=8.4 Hz, 2H), 8.02 (d, J=8.4 Hz, 1H), 9.38 (br s, 1H); ESI-MS: m/z 314.30 (M+H)⁺.

### Example-21: Preparation of (E)-N²-[(2E)-3-(thiophen-3-yl)prop-2-en-1-ylidene]-L-arginine

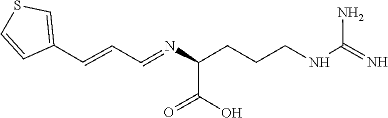 [SMILES: N=C(N)NCCC[C@H](/N=C/C=C/c1ccsc1)C(=O)O]

L-arginine (1.21 g) was added to the solution of (2E)-3-(thiophen-3-yl)prop-2-enal (1 g) in ethanol (20 ml) at room temperature. The reaction mixture was stirred for 18 h at RT under nitrogen atmosphere. The solid product was filtered, washed with n-hexane (20 ml) and then dried under high vacuum to give 1.23 g of (E)-N²-[(2E)-3-(thiophen-3-yl)prop-2-en-1-ylidene]-L-arginine as a solid. ¹H NMR (400 MHz, DMSO-d₆) δ 1.45 (br s, 2H), 1.60-1.63 (m, 1H), 1.81-1.78 (m, 1H), 3.08 (br s, 2H), 3.54 (t, J=6.4 Hz, 1H), 6.70-6.77 (m, 1H), 7.04 (d, J=16.0 Hz, 1H), 7.20-7.60 (br s, 4H), 7.43-7.45 (m, 1H), 7.54-7.59 (m, 1H), 7.71 (s, 1H), 7.95 (d, J=8.8 Hz, 1H), 9.62 (br s, 1H); ESI-MS: m/z 295.21 (M+H)⁺.

### Example-22: Preparation of (E)-N²-[(2E)-3-(1,3-benzothiazol-2-yl)prop-2-en-1-ylidene]-L-arginine

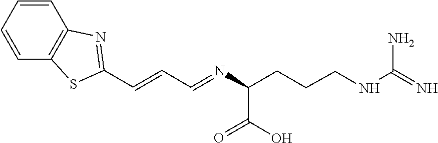 [SMILES: N=C(N)NCCC[C@H](/N=C/C=C/c1nc2ccccc2s1)C(=O)O]

L-arginine (0.44 g) was added to the solution of (2E)-3-(1,3-benzothiazol-2-yl)prop-2-enal (0.5 g) in ethanol (10 ml) at room temperature. The reaction mixture was stirred for 18 h at RT under nitrogen atmosphere. The solid product was filtered, washed with n-hexane (20 ml) and then dried under high vacuum to give 0.81 g of (E)-N²-[(2E)-3-(1,3-benzothiazol-2-yl)prop-2-en-1-ylidene]-L-arginine as a solid. ¹H NMR (400 MHz, DMSO-d₆) δ 1.45 (br s, 2H), 1.62-1.63 (m, 1H), 1.83 (br s, 1H), 3.09-3.16 (br s, 2H), 3.58 (t, J=6.2 Hz, 1H), 6.68-6.74 (m, 1H), 6.95-7.01 (m, 1H), 7.18 (d, J=15.6 Hz, 1H) 7.30 (br, 1H), 7.42-7.54 (m, 4H), 7.96-7.98 (m, 2H), 8.08 (d, J=7.6 Hz, 1H), 9.65 (br s, 1H); ESI-MS: m/z 346.30 (M+H)⁺.

### Example-23: Preparation of (E)-N²-[(2E)-3-(1,3-thiazol-2-yl)prop-2-en-1-ylidene]-L-arginine

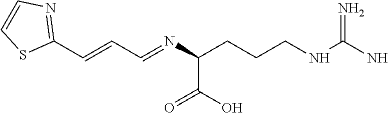 [SMILES: N=C(N)NCCC[C@H](/N=C/C=C/c1nccs1)C(=O)O]

L-arginine (1.19 g) was added to the solution of (2E)-3-(1,3-thiazol-2-yl)prop-2-enal (0.5 g) in ethanol (10 ml) at room temperature. The reaction mixture was stirred for 18 h at RT under nitrogen atmosphere. The solid product was filtered, washed with n-hexane (20 ml) and then dried under high vacuum to give 1.67 g of (E)-N²-[(2E)-3-(1,3-thiazol-2-yl)prop-2-en-1-ylidene]-L-arginine as a solid. ¹H NMR (400 MHz, DMSO-d₆) δ 1.45 (br s, 2H), 1.61-1.70 (m, 1H), 1.82-1.85 (m, 1H), 3.03-3.13 (m, 2H), 3.61 (t, J=6.4 Hz, 1H), 6.96-7.03 (m, 1H), 7.33 (d, J=15.6 Hz, 1H), 7.56 (br s, 4H), 7.81 (d, J=6.4 Hz 1H), 7.93 (d, J=6.2 Hz, 1H), 8.05 (d, J=8.8 Hz, 1H), 9.55 (br s, 1H); ESI-MS: m/z 296.21 (M+H)⁺.

### Example-24: Preparation of (E)-N²-[(2E)-3-(naphthalen-2-yl)prop-2-en-1-ylidene]-L-arginine

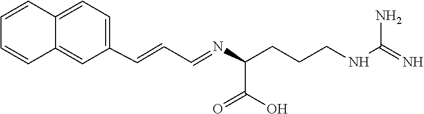 [SMILES: N=C(N)NCCC[C@H](/N=C/C=C/c1ccc2ccccc2c1)C(=O)O]

L-arginine (0.45 g) was added to the solution of (2E)-3-(naphthalen-2-yl)prop-2-enal (0.5 g) in ethanol (10 ml) at room temperature. The reaction mixture was stirred for 18 h at RT under nitrogen atmosphere. The solid product was filtered, washed with n-hexane (20 ml) and then dried under high vacuum to give 0.52 g of (E)-N²-[(2E)-3-(naphthalen-2-yl)prop-2-en-1-ylidene]-L-arginine as a solid. ¹H NMR (400 MHz, DMSO-d₆) δ 1.50 (br s, 2H), 1.65-1.69 (m, 1H), 1.84-1.86 (m, 1H), 3.09 (br s, 2H), 3.59 (t, J=6.4 Hz, 1H), 7.01-7.07 (m, 1H), 7.21 (d, J=16.4 Hz, 1H), 7.30 (br s, 4H), 7.51-7.54 (m, 2H), 7.82 (d, J=8.4 Hz, 1H), 7.90-7.97 (m, 3H), 8.03 (s, 1H), 8.05 (d, J=8.8 Hz, 1H), 9.55 (br s, 1H); ESI-MS: m/z 339.27 (M+H)⁺.

### Example-25: Preparation of (E)-N²-[(2E)-3-(4-methylphenyl)prop-2-en-1-ylidene]-L-arginine

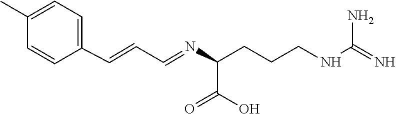 [SMILES: Cc1ccc(/C=C/C=N/[C@@H](CCCNC(=N)N)C(=O)O)cc1]

L-arginine (2.41 g) was added to the solution of (2E)-3-(4-methylphenyl)prop-2-enal (2 g) in ethanol (10 ml) at room temperature. The reaction mixture was stirred for 24 h at RT under nitrogen atmosphere. The solid product was filtered, washed with n-hexane (20 ml) and then dried under high vacuum to give 2.83 g of (E)-N²-[(2E)-3-(4-methylphenyl)prop-2-en-1-ylidene]-L-arginine as a solid. ¹H NMR (400 MHz, DMSO-d₆) δ 1.45 (br s, 2H), 1.65-1.67 (m, 1H), 1.84-1.85 (m, 1H), 2.08 (s, 3H), 3.08 (br s, 2H), 3.60 (t, J=6.2 Hz, 1H), 6.80-6.86 (m, 1H), 6.98 (d, J=15.6 Hz, 1H), 7.20 (d, J=8.0 Hz, 2H), 7.28 (br s, 4H), 7.45 (d, J=8.4 Hz, 2H), 7.95 (d, J=8.4 Hz, 1H), 9.38 (br s, 1H); ESI-MS: m/z 303.30 (M+H)⁺.

The invention is further illustrated by making available the following individual compounds of formula (I) listed below in Tables 1 to 4.

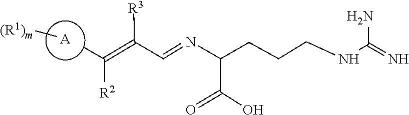

TABLE 1

| Example No. | 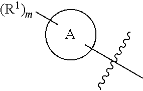 | R² | R³ | ESI-MS: m/z |
|---|---|---|---|---|
| 26 | 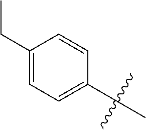 [SMILES: *c1ccc(CC)cc1] | H | H | 317.30 (M + H)⁺ |
| 27 | 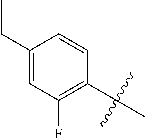 [SMILES: *c1ccc(CC)cc1F] | H | H | 335.20 (M + H)⁺ |
| 28 | 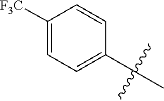 [SMILES: *c1ccc(C(F)(F)F)cc1] | H | H | 356.20 (M)⁺ |
| 29 | 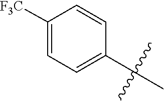 [SMILES: *c1ccc(C(F)(F)F)cc1] | H | CH₃ | 371.20 (M + H)⁺ |
| 30 | 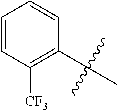 [SMILES: *c1ccccc1C(F)(F)F] | H | H | 357.20 (M + H)⁺ |
| 31 | 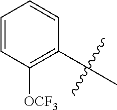 [SMILES: *c1ccccc1OC(F)(F)F] | H | C₂H₅ | 401.30 (M + H)⁺ |
| 32 | 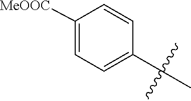 [SMILES: *c1ccc(C(=O)OC)cc1] | H | H | 345.30 (M − H)⁻ |
| 33 | 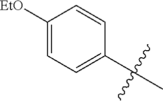 [SMILES: *c1ccc(OCC)cc1] | H | H | 333.40 (M + H)⁺ |
| 34 | 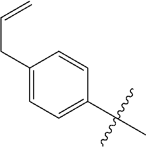 [SMILES: *c1ccc(CC=C)cc1] | H | H | 329.30 (M + H)⁺ |
| 35 | 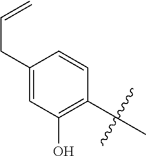 [SMILES: *c1ccc(CC=C)cc1O] | H | H | 343.30 (M − H)⁻ |
| 36 | 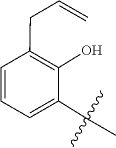 [SMILES: *c1cccc(CC=C)c1O] | H | H | 345.30 (M + H)⁺ |
| 37 | 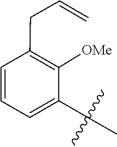 [SMILES: *c1cccc(CC=C)c1OC] | H | H | 359.40 (M + H)⁺ |
| 38 | 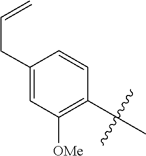 [SMILES: *c1ccc(CC=C)cc1OC] | H | H | 359.30 (M + H)⁺ |
| 39 | 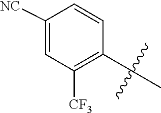 [SMILES: *c1ccc(C#N)cc1C(F)(F)F] | H | H | 382.30 (M + H)⁺ |

TABLE 2

| Example No. | 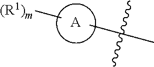 | R² | R³ | ESI-MS: m/z |
|---|---|---|---|---|
| 40 | 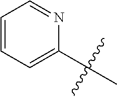 [SMILES: *c1ccccn1] | H | H | 290.20 (M + H)⁺ |
| 41 | 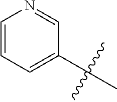 [SMILES: *c1cccnc1] | H | CH₃ | 290.30 (M + H)⁺ |
| 42 | 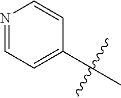 [SMILES: *c1ccncc1] | H | H | 290.20 (M + H)⁺ |
| 43 | 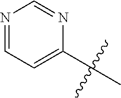 [SMILES: *c1ccncn1] | H | H | 291.30 (M + H)⁺ |
| 44 | 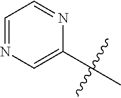 [SMILES: *c1cnccn1] | H | H | 291.30 (M + H)⁺ |
| 45 | 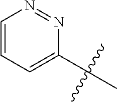 [SMILES: *c1cccnn1] | H | H | 289.20 (M − H)⁻ |

TABLE 3

| Example No. | 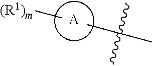 | R² | R³ | ESI-MS: m/z |
|---|---|---|---|---|
| 46 | 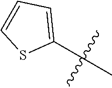 [SMILES: *c1cccs1] | H | CH₃ | 309.30 (M + H)⁺ |
| 47 | 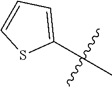 [SMILES: *c1cccs1] | H | C₂H₅ | 323.30 (M + H)⁺ |
| 48 | 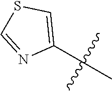 [SMILES: *c1cscn1] | H | C₂H₅ | 324.30 (M + H)⁺ |
| 49 | 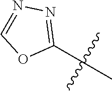 [SMILES: *c1nnco1] | H | H | 289.20 (M + H)⁺ |
| 50 | 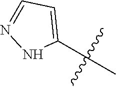 [SMILES: *c1ccn[nH]1] | H | H | 279.20 (M + H)⁺ |
| 51 | 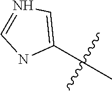 [SMILES: *c1cnc[nH]1] | H | H | 279.30 (M + H)⁺ |
| 52 | 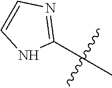 [SMILES: *c1ncc[nH]1] | H | H | 279.20 (M + H)⁺ |
| 53 | 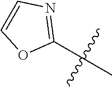 [SMILES: *c1ncco1] | H | H | 280.30 (M + H)⁺ |
| 54 | 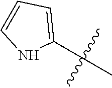 [SMILES: *c1ccc[nH]1] | H | H | 278.20 (M + H)⁺ |

TABLE 4

| Example No. | 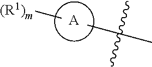 | R² | R³ | ESI-MS: m/z |
|---|---|---|---|---|
| 55 | 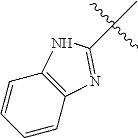 [SMILES: *c1nc2ccccc2[nH]1] | H | H | 329.30 (M + H)⁺ |
| 56 | 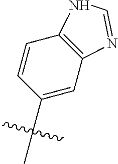 [SMILES: *c1ccc2[nH]cnc2c1] | H | H | 239.20 (M + H)⁺ |
| 57 | 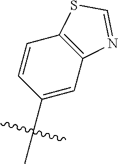 [SMILES: *c1ccc2scnc2c1] | H | H | 346.30 (M + H)⁺ |
| 58 | 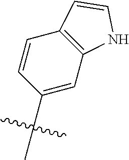 [SMILES: *c1ccc2cc[nH]c2c1] | H | H | 328.30 (M + H)⁺ |
| 59 | 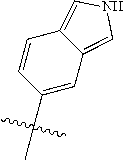 [SMILES: *c1ccc2c[nH]cc2c1] | H | H | 326.20 (M − H)⁻ |
| 60 | 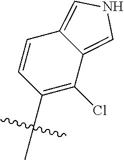 [SMILES: *c1ccc2c[nH]cc2c1Cl] | H | H | 362.70 (M + H)⁺ |
| 61 | 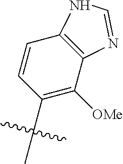 [SMILES: *c1ccc2[nH]cnc2c1OC] | H | H | 359.30 (M + H)⁺ |
| 62 | 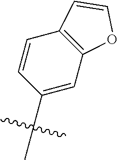 [SMILES: *c1ccc2ccoc2c1] | H | H | 329.30 (M + H)⁺ |
| 63 | 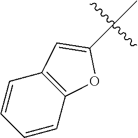 [SMILES: *c1cc2ccccc2o1] | H | H | 329.20 (M + H)⁺ |
| 64 | 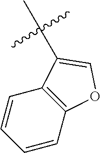 [SMILES: *c1coc2ccccc12] | H | H | 329.30 (M + H)⁺ |
| 65 | 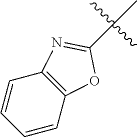 [SMILES: *c1nc2ccccc2o1] | H | H | 329.20 (M + H)⁺ |

### Example-66: Stability Studies of the Compounds of the Present Invention

The compounds of present invention were stored under conditions as shown in below table [room temperature and under accelerated storage condition (ATS)] and the purity was estimated by HPLC. The properly packed material was subjected to the conditions mentioned in the table below (Table 5).

TABLE 5

| | Compound | Day Zero | | Day 14 | |
|---|---|---|---|---|---|
| | of the | Condition | | Condition | |
| S. | present | 25° C. ± | 54° C. ± | 25° C. ± | 54° C. ± |
| No. | invention | 2° C. | 2° C. | 2° C. | 2° C. |
| 1 | Example 3 | 99.23 | 99.10 | 98.80 | 98.79 |
| 2 | Example 9 | 99.60 | 99.63 | 99.59 | 99.64 |
| 3 | Example 20 | 99.55 | 99.43 | 99.50 | 98.90 |
| 4 | Example 24 | 98.90 | 98.75 | 98.85 | 98.90 |
| 5 | Example 39 | 99.40 | 99.40 | 99.55 | 99.60 |

The following examples illustrate the basic methodology and versatility of the composition of the invention. It should be noted that this invention is not limited to these exemplifications. The form of the composition, excipients and the concentrations of the compound of the present invention (Technical) and excipients can be replaced by any other forms, excipients and concentrations as covered in the present invention.

### Example-67: General Procedure for the Preparation of Water Disintegrable Granules (GR)

The compound of the present invention (technical) is ground to desired particle below 50 microns in jet mill, then all the ingredients are added and mixed in ribbon blender to obtain homogeneous powder. Required quantity of water is added to the above mixture to prepare dough and the material is then granulated in suitable equipment and dried to obtain granules.

The following Water Disintegrable Granular (GR) compositions were prepared as per Example 67.

TABLE 6
Formulation (parts by weight)

| Composition No. | I | II | III | IV | V | VI |
|---|---|---|---|---|---|---|
| Technical | Ex. 1 | Ex. 7 | Ex. 10 | Ex. 9 | Ex. 14 | Ex. 36 |
| | 90.6 | 89 | 92 | 90.2 | 83.2 | 87.3 |
| Sodium lauryl sulphate | — | 1.2 | 1.5 | 2 | — | 2 |
| Sodium ligno | — | — | 2 | — | — | — |
| sulphonate | | | | | | |
| Sodium isopropyl | 2 | — | — | — | — | — |
| naphthalene sulfonate | | | | | | |
| Sodium naphthalene | — | — | — | — | 2 | — |
| sulfonate condensate | | | | | | |
| Blend of alkyl | — | — | — | — | 2.5 | — |
| naphthalene sulfonates | | | | | | |
| PEG 400 | — | — | 1 | — | — | 2 |
| Alcohol ethoxylate | 0.5 | — | — | — | — | — |
| Sodium sulphate | — | — | — | 5 | — | — |
| Bentonite | — | — | 3.5 | 2.8 | 6.3 | 6.7 |
| Talc | 1 | 2 | — | — | — | — |
| Corn starch | — | 2.5 | — | — | — | — |
| Perlite | — | — | — | — | 6 | — |
| Mica | — | 5.3 | — | — | — | 2 |
| Silica | 4.9 | — | — | — | — | — |
| Titanium oxide | 1 | — | — | — | — | — |

### Example-68: General Procedure for the Preparation of Extruded Granules (WG)

The compound of the present invention (technical) is blended with dispersing agent, wetting agents and inert excipients in a ribbon blender and then ground to desired particle size in jet mill. Required quantity of water is added to the above mixture to prepare dough and the material is then granulated in suitable equipment and dried to obtain granules.

The following Extruded Granular (WG) compositions were prepared as per Example 68.

TABLE 7
Formulation (parts by weight)

| Composition No. | I | II | III | IV | V | VI | VII |
|---|---|---|---|---|---|---|---|
| Technical | Ex. 1 | Ex. 7 | Ex. 9 | Ex. 10 | Ex. 43 | Ex. 20 | Ex. 63 |
| | 52.7 | 40.9 | 45 | 50.5 | 30.5 | 40.8 | 30.2 |
| Sodium alkyl naphthalene sulfonate | 1 | 2 | — | — | — | — | — |
| Sodium naphthalene sulfonate condensate | 6 | 4 | — | — | 3.5 | — | — |
| Sodium sulphate | 30.3 | 28.1 | — | — | 32.3 | — | 20 |
| Sodium ligno sulphonate | — | 5 | — | 7 | 7 | 8 | — |
| Polycarboxylate | — | — | 3 | — | — | — | 4 |
| Sodium lauryl sulphate | — | — | — | 2 | — | — | 2 |
| Ethoxylated polyarylphenol phosphate amine salt | — | — | 2 | — | — | — | 1 |
| Polyalkoxylated butyl ether | — | — | 7 | — | — | — | — |
| Dioctyl Sodium Sulfosuccinate | — | — | — | — | — | 3 | — |
| Tristyryl phenol phosphate | — | — | — | — | 2.5 | — | — |
| Silicon defoamer | — | — | — | — | 0.2 | — | — |
| lactose | — | — | 18 | 18 | — | 20 | 20 |
| Sodium citrate | — | — | — | 2 | — | — | — |
| Maltodextrin | — | 10 | — | — | — | — | — |
| Clay | 10 | 5 | — | — | 10 | — | — |
| Fumed silica | — | — | — | — | 2 | — | — |
| Soapstone clay | — | — | 25 | — | — | — | — |
| Perlite | — | — | — | 15 | 12 | 28.2 | 22.8 |
| Aluminium silicate | — | 5 | — | — | — | — | — |
| Mica | — | — | — | 5.5 | — | — | — |

### Example-69: General Procedure for the Preparation of Spray Dried Granules (WG)

The compound of the present invention (technical) is blended with all the ingredients in 100 parts of water and milled to desired particle size. The milled slurry is then spray dried/fluid bed dried to obtain granules.

The following spray dried granular (WG) compositions were prepared as per Example 69.

TABLE 8
Formulation (parts by weight)

| Composition No. | I | II | III | IV | V | VI |
|---|---|---|---|---|---|---|
| Technical | Ex. 14 | Ex. 23 | Ex. 9 | Ex. 1 | Ex. 20 | Ex. 10 |
| | 67 | 75.8 | 71.2 | 70.5 | 60.5 | 73.2 |
| Sodium alkyl | 2 | — | — | — | — | — |
| naphthalene | | | | | | |
| sulfonate | | | | | | |
| Sodium ligno | 15 | 10 | 9 | 10 | — | — |
| sulphonate | | | | | | |
| Modified ligno | — | — | — | — | 8 | — |
| sulphonate | | | | | | |
| Sodium sulphate | — | — | 10 | — | — | — |
| Sodium naphthalene | 1 | — | 2 | — | 5 | 6 |
| sulfonate condensate | | | | | | |
| Naphthalene | — | — | — | 2 | — | — |
| Sulphonic | | | | | | |
| Acid and Phenol | | | | | | |
| Sulphonic Acid | | | | | | |
| condensation | | | | | | |
| 4-4 dihydroxy | — | — | — | 3 | — | — |
| diphenyl | | | | | | |
| sulphonate | | | | | | |
| condensate | | | | | | |
| Ethoxylated | — | 3 | — | — | — | — |
| polyarylphenol | | | | | | |
| phosphate amine | | | | | | |
| salt | | | | | | |
| Polyvinyl alcohol | — | — | — | — | 0.2 | — |
| (PVA) | | | | | | |
| Sodium | — | — | — | — | 2 | — |
| hexametaphosphate | | | | | | |
| Sodium | — | — | — | — | — | 1.5 |
| tripolyphosphate | | | | | | |
| Polycarboxylate | — | 1.5 | — | — | — | — |
| Lactose | — | — | — | — | — | 6 |
| Sodium citrate | — | — | — | 2 | — | — |
| Maltodextrin | 5 | — | — | — | — | — |
| Modified starch | — | — | — | 0.5 | — | — |
| Clay | 10 | — | — | — | — | — |
| Kaolin clay | — | 9.7 | 5.8 | 12 | 24.3 | 13.3 |
| Fumed silica | — | — | 2 | — | — | — |

### Example-70: General Procedure for the Preparation of Wettable Powder (WP)

The compound of the present invention (technical) is blended with required quantity of dispersing agent, wetting agents and inert ingredients in a ribbon blender. Blended material is passed through jet mill to reduce the particle size and further blended for one hour to obtain product of desired composition.

The following Wettable Powder (WP) compositions were prepared as per Example 70.

TABLE 9
Formulation (parts by weight)

| Composition No. | I | II | III | IV | V | VI |
|---|---|---|---|---|---|---|
| Technical | Ex. 39 | Ex. 9 | Ex. 50 | Ex. 1 | Ex. 10 | Ex. 7 |
| | 90.2 | 83.5 | 70.6 | 52.4 | 64.1 | 61 |
| Sodium alkyl | 2.8 | 2 | 2.5 | — | — | — |
| naphthalene sulfonate | | | | | | |
| Sodium ligno | 6 | 4 | — | 12 | — | 10 |
| sulphonate | | | | | | |
| Sodium naphthalene | — | — | 4 | 5 | 7 | 6 |
| sulfonate condensate | | | | | | |
| Blend of alkyl | — | — | — | — | 4 | — |
| naphthalene sulfonate | | | | | | |
| Polycarboxylate | — | 1.5 | — | 1 | — | — |
| Sodium lauryl | — | — | — | 4 | — | 4 |
| sulphate | | | | | | |
| Sodium sulphate | — | — | — | — | — | 4 |
| Clay | 1 | — | — | — | — | — |
| Silica | — | 3.2 | 4 | 4 | 6 | 5 |
| Kaolin clay | — | 5.8 | 18.9 | 21.6 | 18.9 | 10 |

### Example-71: General Procedure for the Preparation of Suspension Concentrate (SC)

Required quantity of surfactant and anti-freezing agent is mixed with defined quantity of water. The compound of the present invention (technical) is further added to the homogenized blend and it is stirred continuously for approximately 20 minutes until the total mixture is homogeneous. Half quantity of defoamer is added under continuous homogenization to obtain liquid suspension. Subsequently, the suspension obtained is passed through the wet mill to reduce the particle size. The required qty of thickener, preservative and balance water and defoamer is added to get the product of desired composition.

### Example-72: General Procedure for the Preparation of Coated Granules (GR)

Required quantity of stabilizer, surfactant, coloring agent, binder is blended and heated to desired temperature in drum coater Required quantity of river sand is charged into the drum coater and mixed. The compound of the present invention (technical) of desired particle size is charged into the ribbon blender and mixed to obtain product of desired composition.

### Example-73: General Procedure for the Preparation of Powder for Dry Seed Treatment (DS)

The compound of the present invention (technical) is blended with required quantity of pigment, wetting agent and inert ingredients in a ribbon blender. Blended material is passed through jet mill to reduce the particle size and further blended for one hour to obtain product of desired composition.

### Example-74: General Procedure for the Preparation of Water Dispersible Powder for Slurry Treatment (WS)

The compound of the present invention (technical) is blended with required quantity of pigment, dispersing agent, wetting agents and inert ingredients in a ribbon blender. Blended material is passed through jet mill to reduce the particle size and further blended for one hour to obtain product of desired composition.

The following Suspension concentrate (SC), Coated granules (GR), Powder for dry seed treatment (DS) and Water dispersible powder for slurry treatment (WS) compositions were prepared as per Examples 71, 72, 73 and 74 respectively.

TABLE 10

| | | | | | Powder for | | Water dispersible | |
|---|---|---|---|---|---|---|---|---|
| | Suspension | | Coated | | dry seed | | powder for | |
| | concentrate | | granules | | treatment | | slurry treatment | |
| Formulation (parts by weight) | (SC) | | (GR) | | (DS) | | (WS) | |
| Technical | Ex. 1 | Ex. 9 | Ex. 9 | Ex. 10 | Ex. 20 | Ex. 43 | Ex. 24 | Ex. 7 |
| | 10.6 | 15.2 | 4.5 | 20 | 52.8 | 40.4 | 52.8 | 30.3 |
| Isodecyl alcohol polyglycol ether | — | — | 3 | 2 | — | — | — | — |
| Polyvinyl alcohol (PVA) | — | — | 0.5 | — | — | — | — | — |
| 1,2-benzisothiazolin-3-one | 0.1 | 0.5 | — | — | — | — | — | — |
| Anionic tristyrylphenol phosphate | 3.5 | 4.5 | — | — | — | — | — | — |
| Sodium naphthalene sulfonate condensate | 2 | — | — | — | — | 3 | 3 | 4 |
| Sodium alkyl naphthalene sulfonate | — | 2.5 | — | — | 3.5 | — | 2 | — |
| Sodium ligno sulphonate | — | — | — | — | 1 | 3 | 8 | 4 |
| Silicon defoamer | 0.5 | 0.5 | — | — | — | — | — | — |
| Propylene glycol | 5 | 5 | — | 2 | — | — | — | — |
| Xantham gum | 0.12 | 0.12 | — | | — | — | — | — |
| River sand | — | — | 91.2 | 75.2 | — | — | — | — |
| Silica | — | — | — | — | 2 | — | 2 | — |
| Clay | — | — | — | — | 36.7 | 50.6 | 29.2 | 57.7 |
| Pigment | — | — | — | — | 4 | 3 | 3 | 4 |
| Dye | — | — | 0.8 | 0.8 | — | — | — | — |
| Water | 78.18 | 71.68 | — | — | — | — | — | — |

### Example-75: Determination of the Nematocidal Activity Against Root-Knot Nematodes (Meloidogyne Spp)

The compound to be tested (4 mg) was dissolved in 40 μl of DMSO to make 100,000 PPM concentration stock solution. The clear solution was obtained by vortexing. The nematode suspension (250 μl) having 70-100 juveniles (J2) of Root-knot nematodes Meloidogyne spp. pipetted out to each cell of 24 well bioassay plate. Sterile water (1 ml) was pipetted out in fresh 2.5 ml Eppendorf micro-centrifuge tubes. The desired concentration (500 ppm) of the compound was prepared by adding stock solution in 2.5 ml Eppendorf micro-centrifuge tubes. For each treatment 4 replications were maintained. The bioassay plates were incubated at 22±2° C. and 70±5% RH in humidity chambers. The Dead/inactive nematodes were counted under microscope at 24, 48, 72 and 96 hours after incubation and the percent mortality is calculated.

TABLE 11
Rating Table

| % Mortality | Rating |
|---|---|
| 70-100 | A |
| 50-69 | B |
| 0 to 49 | C |

TABLE 12
Biological results

| Example No. | Rating |
|---|---|
| 1 | A |
| 2 | B |
| 3 | B |
| 4 | B |
| 5 | B |
| 6 | A |
| 7 | A |
| 8 | C |
| 9 | A |
| 10 | A |
| 11 | A |
| 12 | C |
| 13 | C |
| 14 | A |
| 15 | B |
| 16 | C |
| 17 | A |
| 18 | A |
| 19 | C |
| 20 | A |
| 21 | A |
| 22 | A |
| 23 | B |
| 24 | B |
| 25 | A |
| 26 | A |
| 27 | A |
| 28 | B |
| 29 | B |
| 30 | B |
| 31 | C |
| 32 | C |
| 33 | C |
| 34 | B |
| 35 | B |
| 36 | A |
| 37 | B |
| 38 | B |
| 39 | A |
| 40 | A |
| — | — |
| — | — |
| 41 | A |
| 42 | A |
| 43 | A |
| 44 | B |
| 45 | C |
| 46 | B |
| 47 | B |
| 48 | B |
| 49 | C |
| 50 | B |
| 51 | B |
| 51 | B |
| 52 | B |
| 53 | C |
| 54 | B |
| 55 | C |
| 56 | B |
| 57 | A |
| 58 | A |
| 59 | A |
| 60 | B |
| 61 | C |
| 62 | B |
| 63 | A |
| 64 | B |
| 65 | B |
| — | — |

1-26. (canceled)

27. A compound of formula (I) 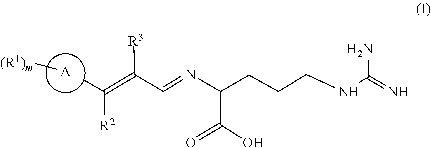
or an agrochemically acceptable salt, stereoisomer, enantiomer, tautomer, or N-oxide thereof,
wherein:
ring A is selected from phenyl, naphthyl, pyridinyl, thienyl, furyl, thiazolyl, oxazolyl, isoxazolyl, oxadiazolyl, pyrrolyl, pyrazolyl, imidazolyl, pyrimidinyl, indolyl, isoindolyl, indazolyl, benzofuryl, benzoxazolyl, and benzothiozolyl;
at each occurrence, R¹ is selected from hydrogen, halogen, —OH, —CN, C₁₋₆alkyl, C₁₋₆alkoxy, haloC₁₋₆alkyl, haloC₁₋₆alkoxy, C₂₋₆alkenyl, —CO(O)—C₁₋₆alkyl, —OC(O)—C₁₋₆alkyl and —OC(O)—C₆₋₁₄aryl;
R² and R³ is independently hydrogen, or C₁₋₆alkyl; and
m is an integer ranging from 0 to 4, both inclusive.

28. The compound according to claim 27, wherein

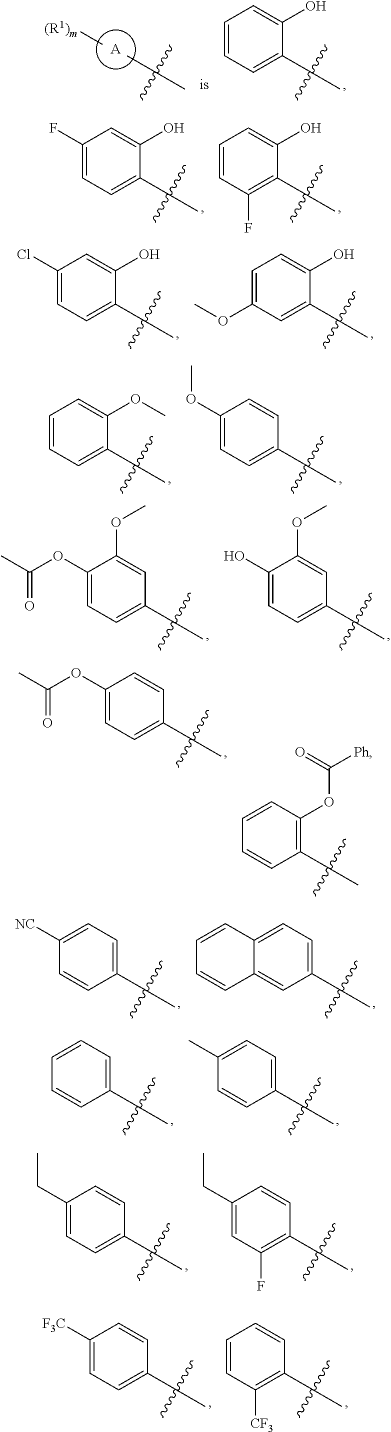

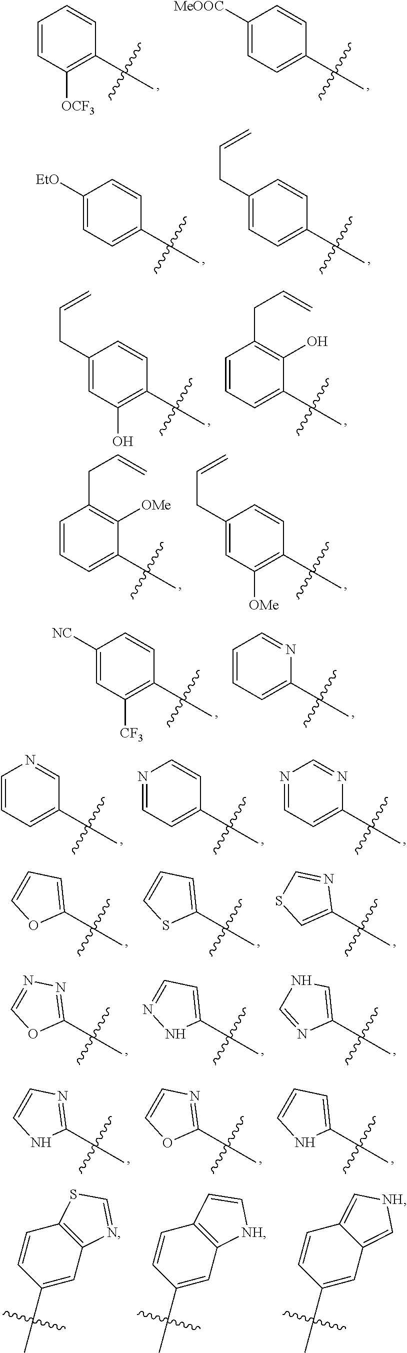

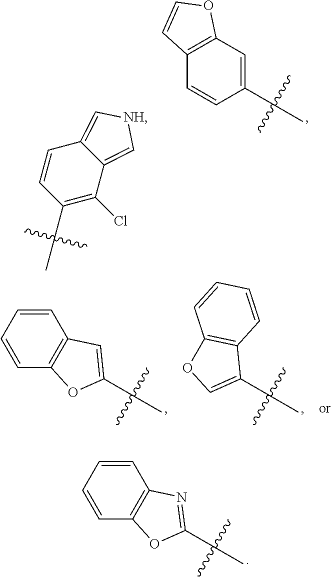 [SMILES: *c1ccc2ccoc2c1 ; *c1ccc2c[nH]cc2c1Cl ; *c1cc2ccccc2o1 ; *c1coc2ccccc12 ; *c1nc2ccccc2o1]

29. The compound according to claim 27 wherein 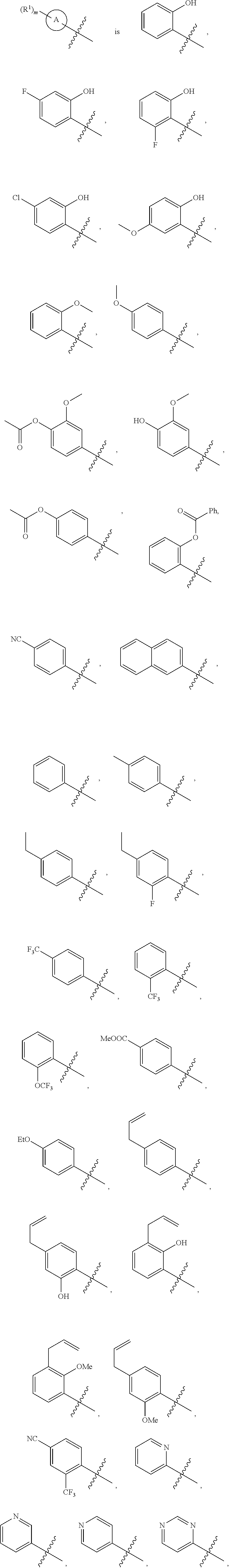 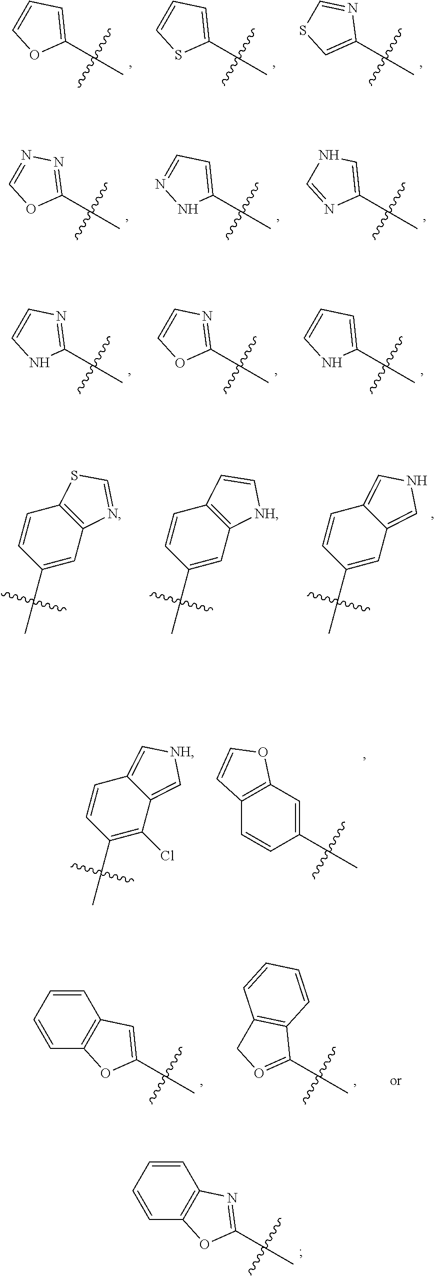
R² is hydrogen; and
R³ is hydrogen, methyl, ethyl, pentyl or hexyl.

30. The compound according to claim 27, wherein the compound is selected from 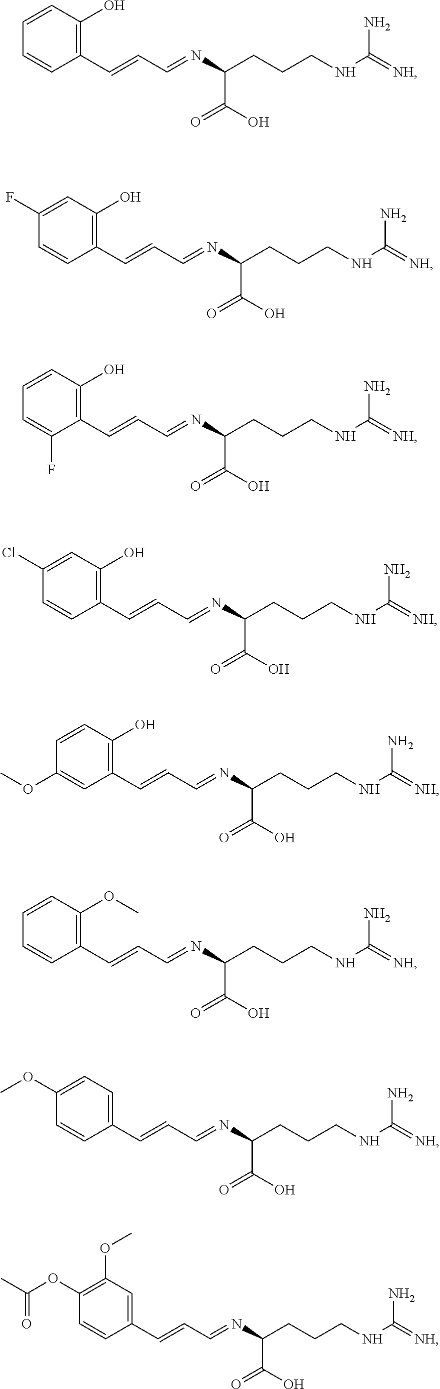 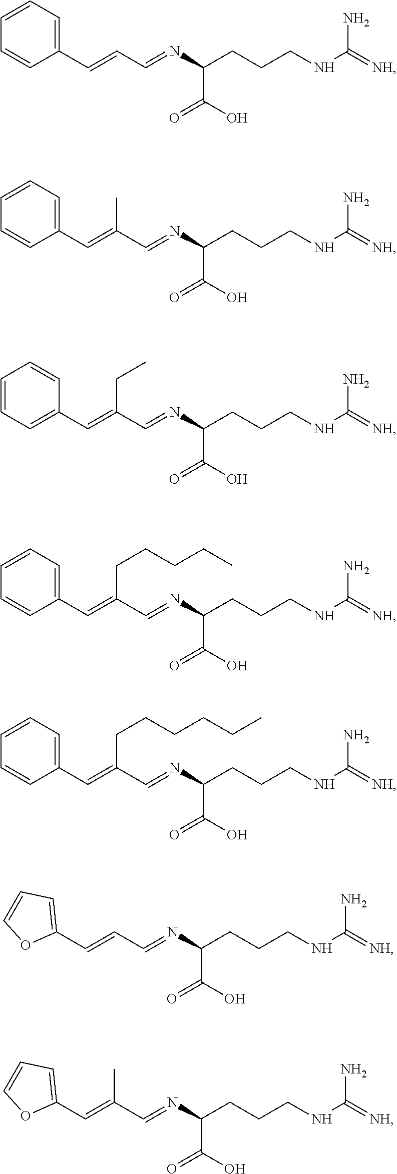 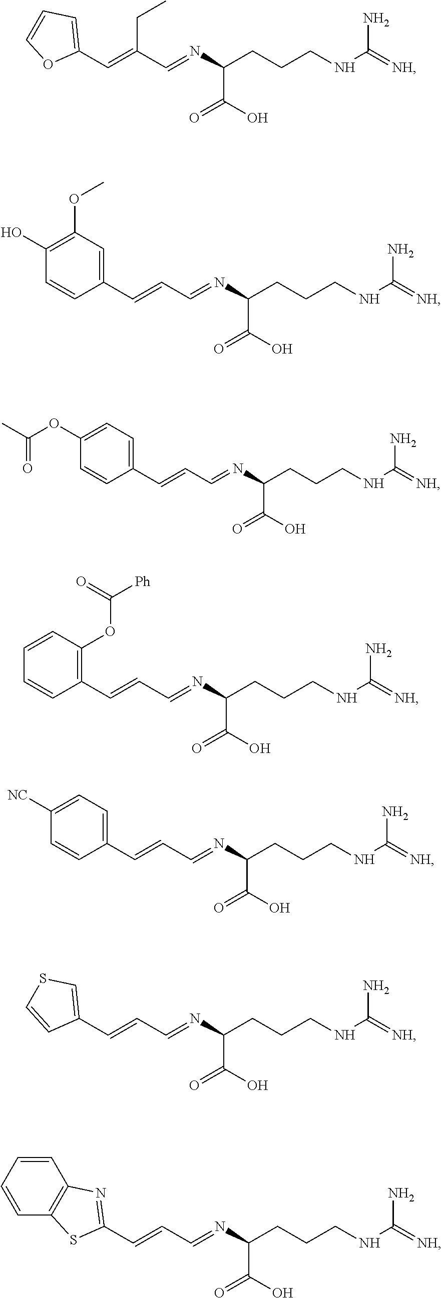 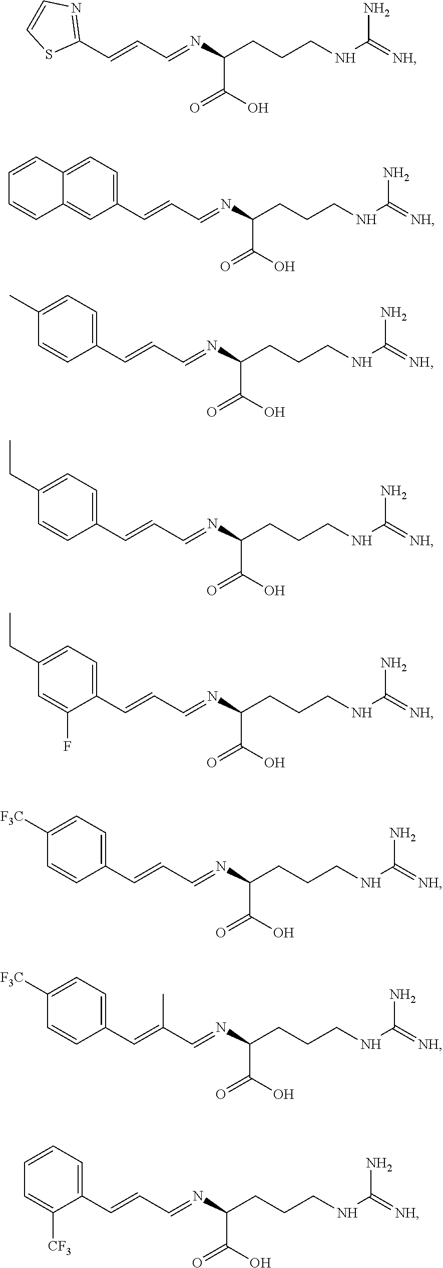 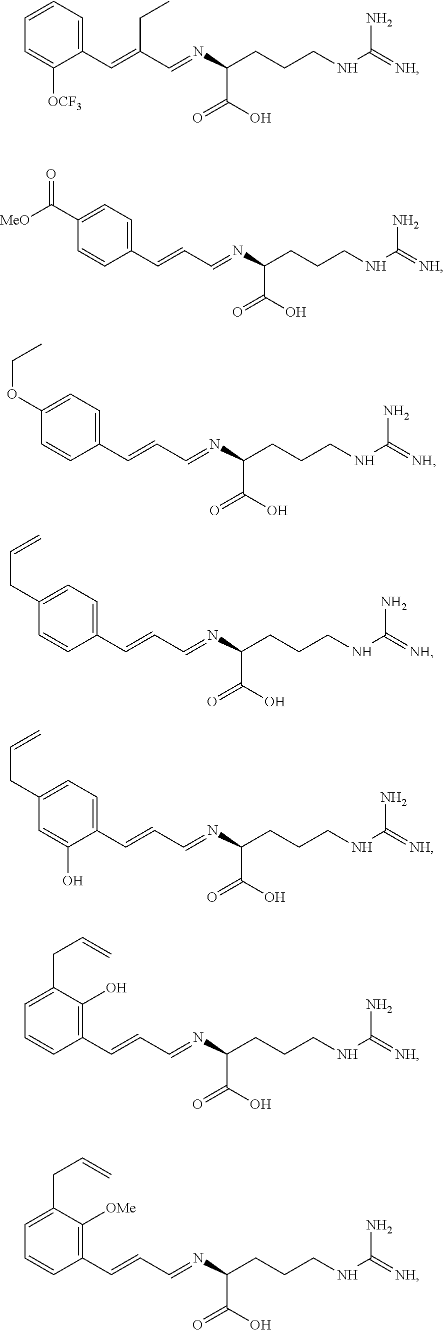 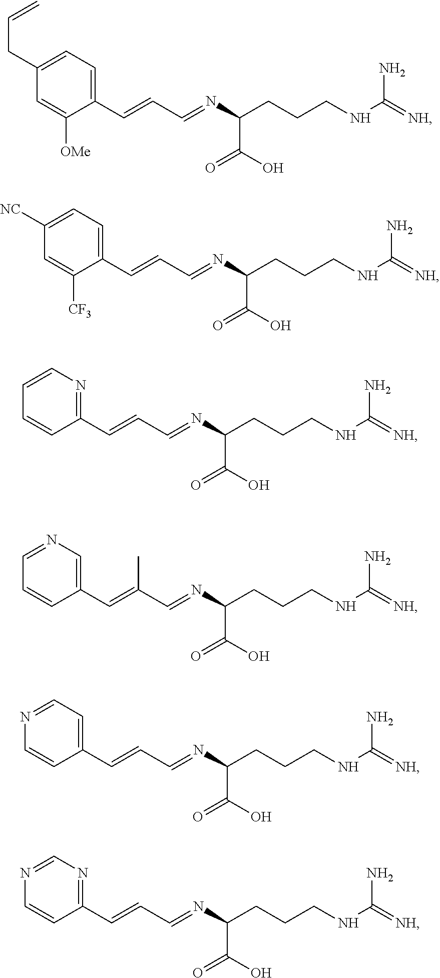 [SMILES: C=CCc1ccc(/C=C/C=N/[C@@H](CCCNC(=N)N)C(=O)O)c(OC)c1 ; N#Cc1ccc(/C=C/C=N/[C@@H](CCCNC(=N)N)C(=O)O)c(C(F)(F)F)c1 ; N=C(N)NCCC[C@H](/N=C/C=C/c1ccccn1)C(=O)O ; CC(/C=N/[C@@H](CCCNC(=N)N)C(=O)O)=C\c1cccnc1 ; N=C(N)NCCC[C@H](/N=C/C=C/c1ccncc1)C(=O)O ; N=C(N)NCCC[C@H](/N=C/C=C/c1ccncn1)C(=O)O] 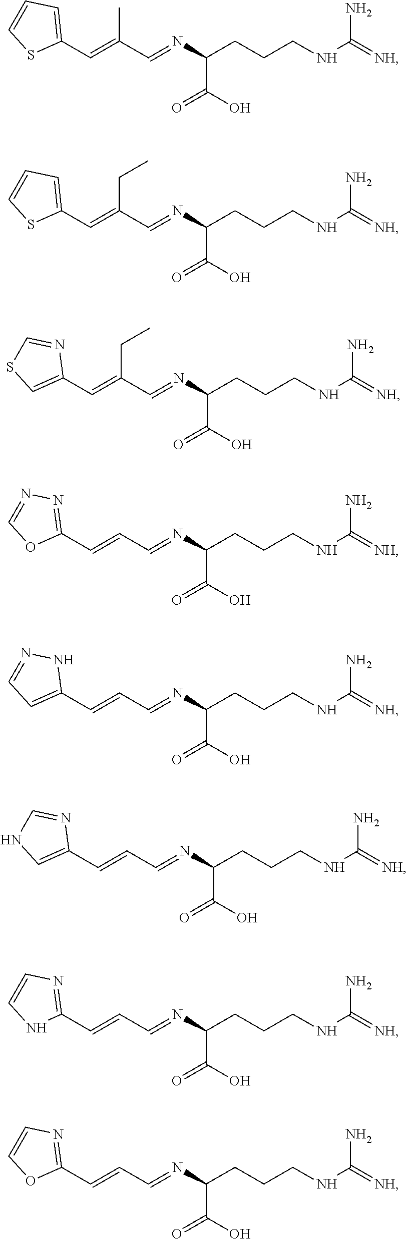 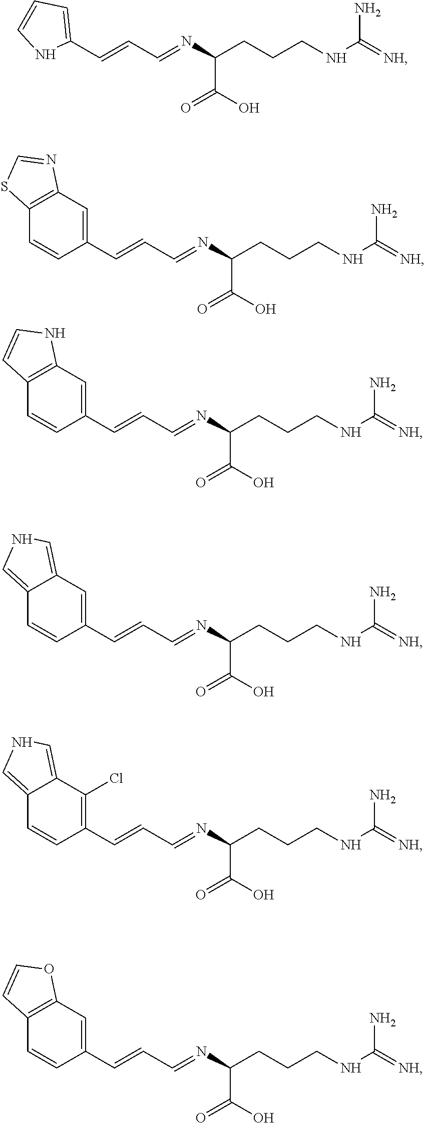 [SMILES: N=C(N)NCCC[C@H](/N=C/C=C/c1ccc[nH]1)C(=O)O ; N=C(N)NCCC[C@H](/N=C/C=C/c1ccc2scnc2c1)C(=O)O ; N=C(N)NCCC[C@H](/N=C/C=C/c1ccc2cc[nH]c2c1)C(=O)O ; N=C(N)NCCC[C@H](/N=C/C=C/c1ccc2c[nH]cc2c1)C(=O)O ; N=C(N)NCCC[C@H](/N=C/C=C/c1ccc2c[nH]cc2c1Cl)C(=O)O ; N=C(N)NCCC[C@H](/N=C/C=C/c1ccc2ccoc2c1)C(=O)O] 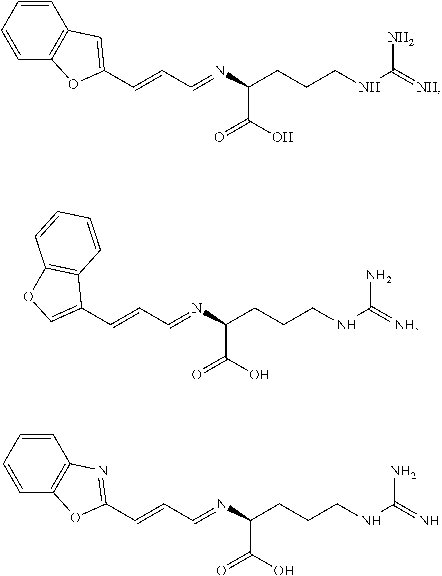 [SMILES: N=C(N)NCCC[C@H](/N=C/C=C/c1cc2ccccc2o1)C(=O)O ; N=C(N)NCCC[C@H](/N=C/C=C/c1coc2ccccc12)C(=O)O ; N=C(N)NCCC[C@H](/N=C/C=C/c1nc2ccccc2o1)C(=O)O]
or agrochemically acceptable salts or N-oxides thereof.

31. The compound according to claim 27, which is 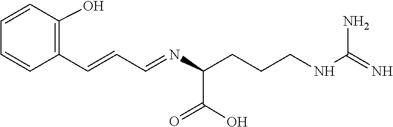 [SMILES: N=C(N)NCCC[C@H](/N=C/C=C/c1ccccc1O)C(=O)O]
or agrochemically acceptable salts or N-oxides thereof.

32. The compound according to claim 27, which is 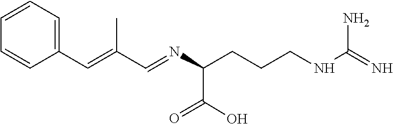 [SMILES: CC(/C=N/[C@@H](CCCNC(=N)N)C(=O)O)=C\c1ccccc1]
or agrochemically acceptable salts or N-oxides thereof.

33. The compound according to claim 27, which is 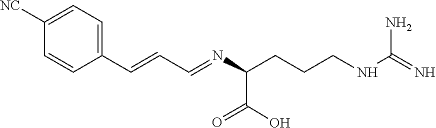 [SMILES: N#Cc1ccc(/C=C/C=N/[C@@H](CCCNC(=N)N)C(=O)O)cc1]
or agrochemically acceptable salts or N-oxides thereof.

34. The compound according to claim 27, which is 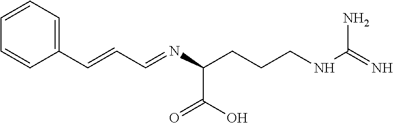 [SMILES: N=C(N)NCCC[C@H](/N=C/C=C/c1ccccc1)C(=O)O]
or agrochemically acceptable salts or N-oxides thereof.

35. The compound according to claim 27, which is 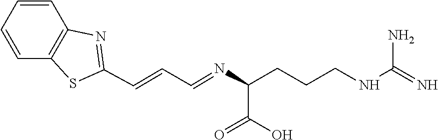 [SMILES: N=C(N)NCCC[C@H](/N=C/C=C/c1nc2ccccc2s1)C(=O)O]
or agrochemically acceptable salts or N-oxides thereof.

36. An agricultural composition comprising the compound of claim 27, and at least one agrochemically acceptable excipient.

37. The agricultural composition according to claim 36, wherein the agrochemically acceptable excipient is selected from one or more of surfactants, disintegrating agents, fillers or carriers or diluents, spreading agents, colorants, anticaking agents, binders, buffers or pH adjusters or neutralizing agents, pigments, stabilizers, antifoaming agents or defoamers, penetrants, structuring agents, humectants, sticking agents, anti-freezing agent or freeze point depressants, chelating or complexing or sequestering agents, or preservatives.

38. The agricultural composition according to claim 36, wherein the compound is selected from 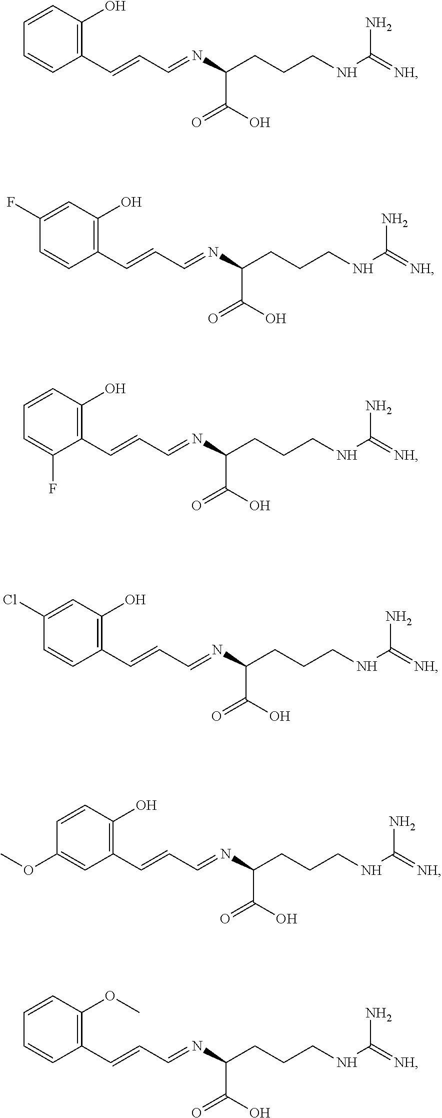 [SMILES: N=C(N)NCCC[C@H](/N=C/C=C/c1ccccc1O)C(=O)O ; N=C(N)NCCC[C@H](/N=C/C=C/c1ccc(F)cc1O)C(=O)O ; N=C(N)NCCC[C@H](/N=C/C=C/c1c(O)cccc1F)C(=O)O ; N=C(N)NCCC[C@H](/N=C/C=C/c1ccc(Cl)cc1O)C(=O)O ; COc1ccc(O)c(/C=C/C=N/[C@@H](CCCNC(=N)N)C(=O)O)c1 ; COc1ccccc1/C=C/C=N/[C@@H](CCCNC(=N)N)C(=O)O] 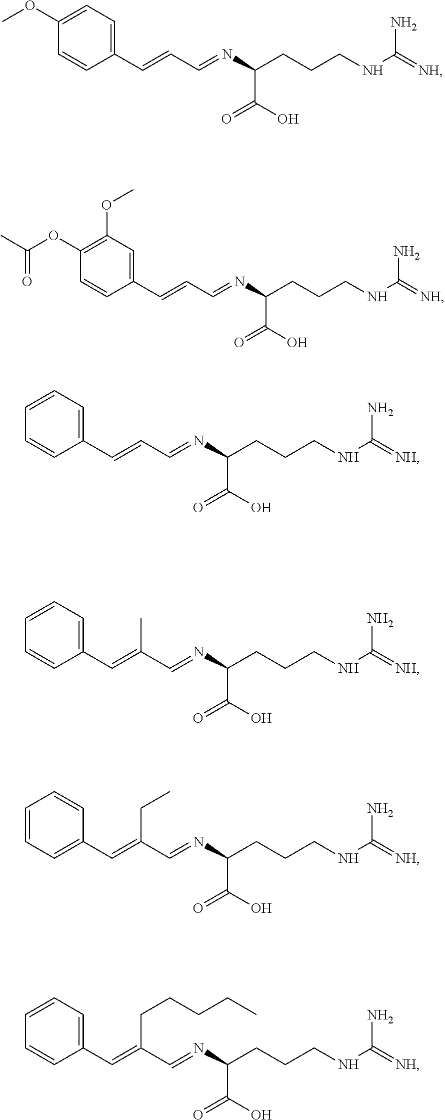 [SMILES: COc1ccc(/C=C/C=N/[C@@H](CCCNC(=N)N)C(=O)O)cc1 ; COc1cc(/C=C/C=N/[C@@H](CCCNC(=N)N)C(=O)O)ccc1OC(C)=O ; N=C(N)NCCC[C@H](/N=C/C=C/c1ccccc1)C(=O)O ; CC(/C=N/[C@@H](CCCNC(=N)N)C(=O)O)=C\c1ccccc1 ; CCC(/C=N/[C@@H](CCCNC(=N)N)C(=O)O)=C\c1ccccc1 ; CCCCCC(/C=N/[C@@H](CCCNC(=N)N)C(=O)O)=C\c1ccccc1] 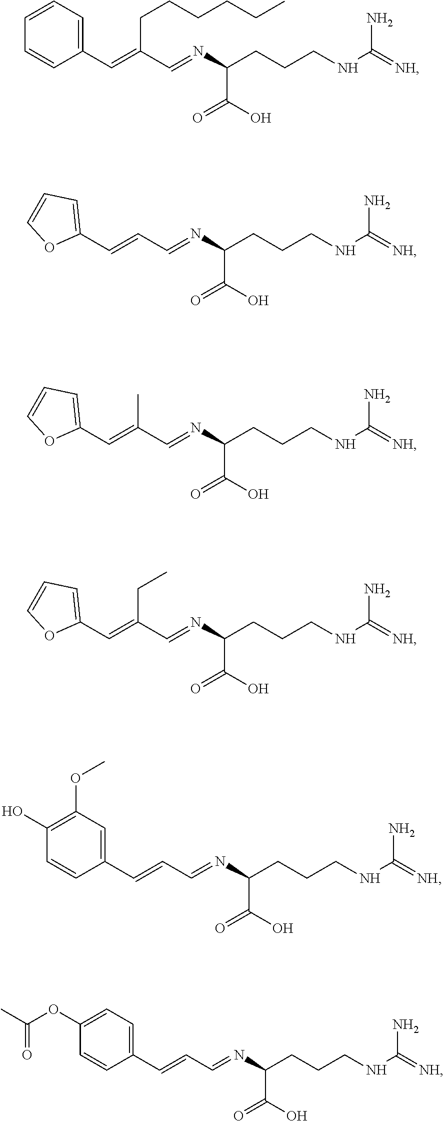 [SMILES: CCCCCCC(/C=N/[C@@H](CCCNC(=N)N)C(=O)O)=C\c1ccccc1 ; N=C(N)NCCC[C@H](/N=C/C=C/c1ccco1)C(=O)O ; CC(/C=N/[C@@H](CCCNC(=N)N)C(=O)O)=C\c1ccco1 ; CCC(/C=N/[C@@H](CCCNC(=N)N)C(=O)O)=C\c1ccco1 ; COc1cc(/C=C/C=N/[C@@H](CCCNC(=N)N)C(=O)O)ccc1O ; CC(=O)Oc1ccc(/C=C/C=N/[C@@H](CCCNC(=N)N)C(=O)O)cc1] 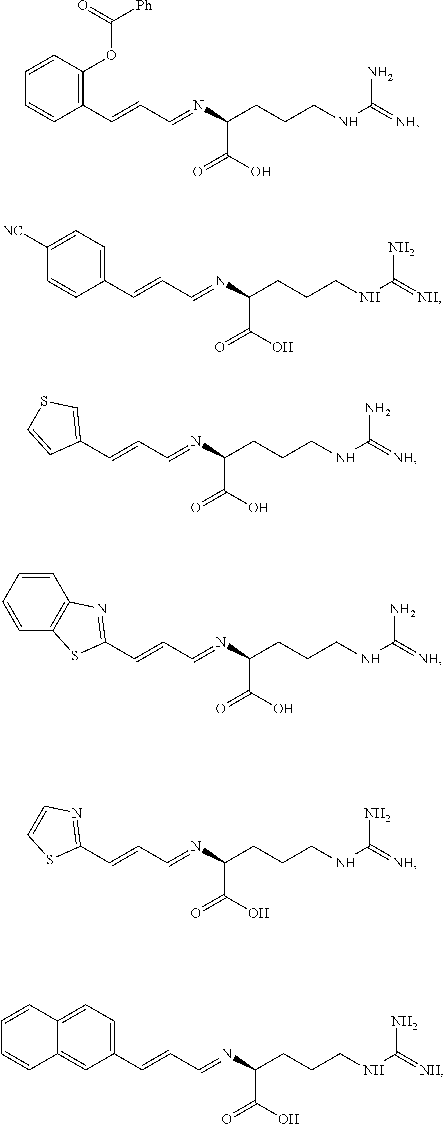 [SMILES: N=C(N)NCCC[C@H](/N=C/C=C/c1ccccc1OC(=O)c1ccccc1)C(=O)O ; N#Cc1ccc(/C=C/C=N/[C@@H](CCCNC(=N)N)C(=O)O)cc1 ; N=C(N)NCCC[C@H](/N=C/C=C/c1ccsc1)C(=O)O ; N=C(N)NCCC[C@H](/N=C/C=C/c1nc2ccccc2s1)C(=O)O ; N=C(N)NCCC[C@H](/N=C/C=C/c1nccs1)C(=O)O ; N=C(N)NCCC[C@H](/N=C/C=C/c1ccc2ccccc2c1)C(=O)O] 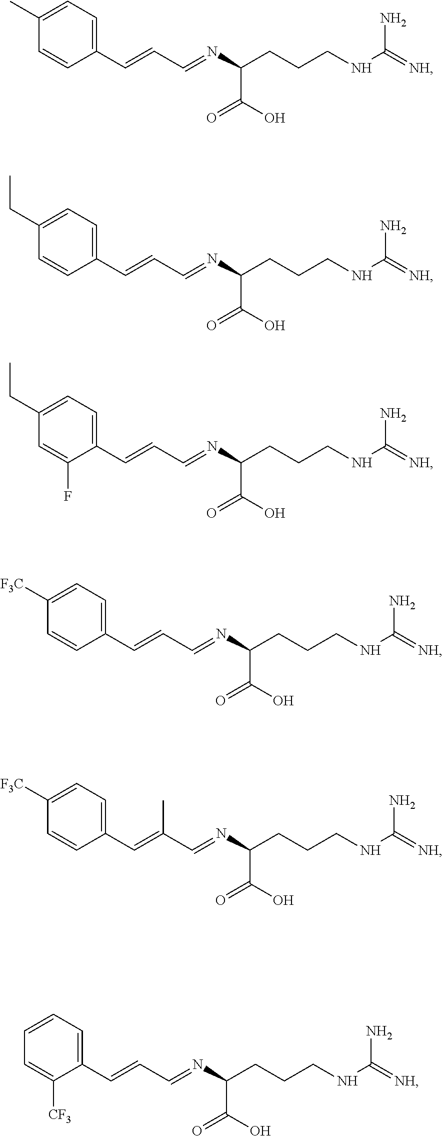 [SMILES: Cc1ccc(/C=C/C=N/[C@@H](CCCNC(=N)N)C(=O)O)cc1 ; CCc1ccc(/C=C/C=N/[C@@H](CCCNC(=N)N)C(=O)O)cc1 ; CCc1ccc(/C=C/C=N/[C@@H](CCCNC(=N)N)C(=O)O)c(F)c1 ; N=C(N)NCCC[C@H](/N=C/C=C/c1ccc(C(F)(F)F)cc1)C(=O)O ; CC(/C=N/[C@@H](CCCNC(=N)N)C(=O)O)=C\c1ccc(C(F)(F)F)cc1 ; N=C(N)NCCC[C@H](/N=C/C=C/c1ccccc1C(F)(F)F)C(=O)O] 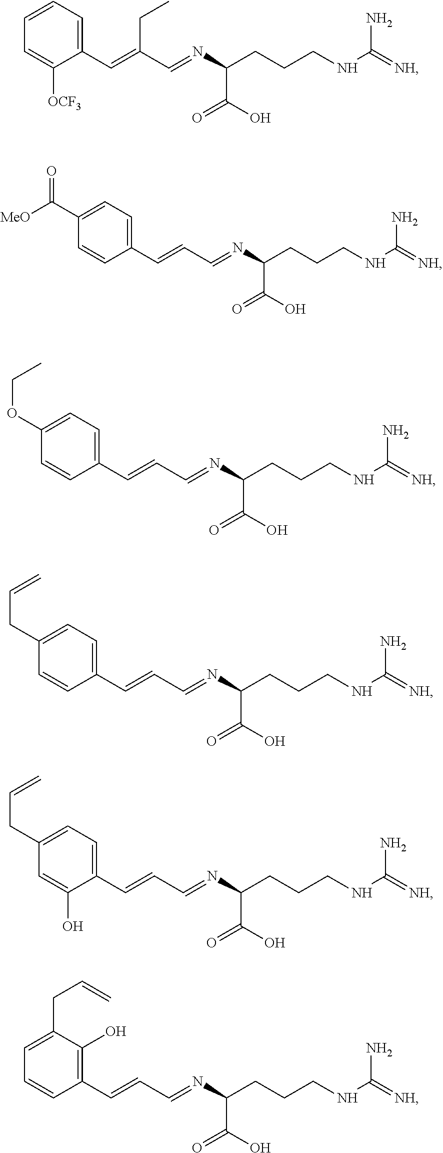 [SMILES: CCC(/C=N/[C@@H](CCCNC(=N)N)C(=O)O)=C\c1ccccc1OC(F)(F)F ; COC(=O)c1ccc(/C=C/C=N/[C@@H](CCCNC(=N)N)C(=O)O)cc1 ; CCOc1ccc(/C=C/C=N/[C@@H](CCCNC(=N)N)C(=O)O)cc1 ; C=CCc1ccc(/C=C/C=N/[C@@H](CCCNC(=N)N)C(=O)O)cc1 ; C=CCc1ccc(/C=C/C=N/[C@@H](CCCNC(=N)N)C(=O)O)c(O)c1 ; C=CCc1cccc(/C=C/C=N/[C@@H](CCCNC(=N)N)C(=O)O)c1O] 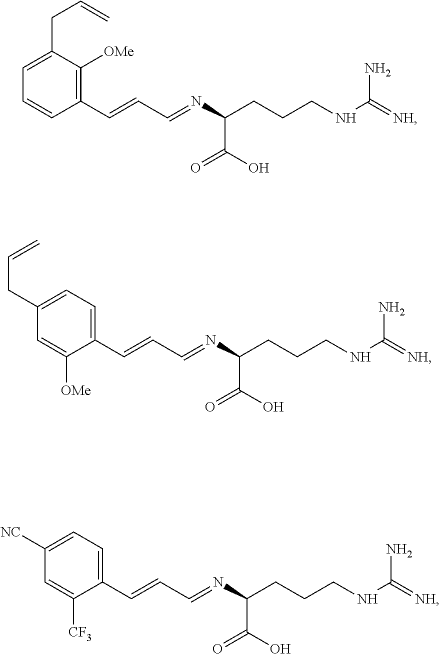 [SMILES: C=CCc1cccc(/C=C/C=N/[C@@H](CCCNC(=N)N)C(=O)O)c1OC ; C=CCc1ccc(/C=C/C=N/[C@@H](CCCNC(=N)N)C(=O)O)c(OC)c1 ; N#Cc1ccc(/C=C/C=N/[C@@H](CCCNC(=N)N)C(=O)O)c(C(F)(F)F)c1] 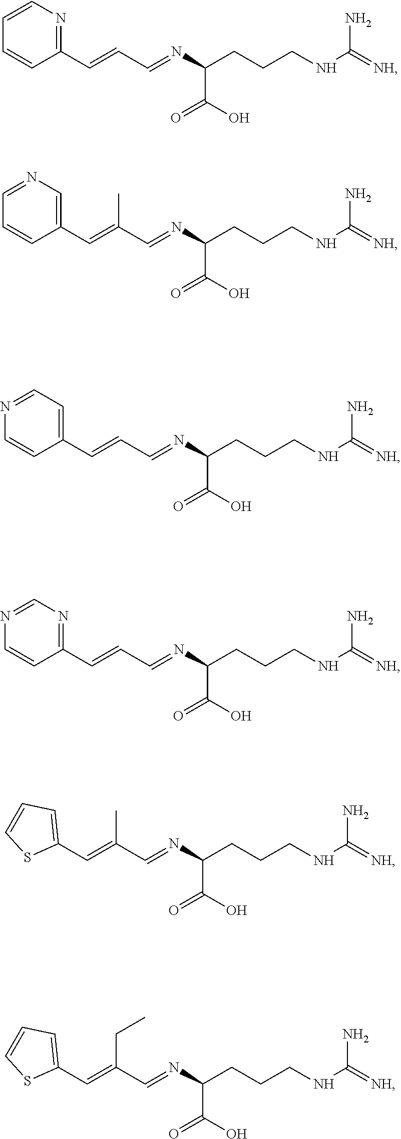 [SMILES: N=C(N)NCCC[C@H](/N=C/C=C/c1ccccn1)C(=O)O ; CC(/C=N/[C@@H](CCCNC(=N)N)C(=O)O)=C\c1cccnc1 ; N=C(N)NCCC[C@H](/N=C/C=C/c1ccncc1)C(=O)O ; N=C(N)NCCC[C@H](/N=C/C=C/c1ccncn1)C(=O)O ; CC(/C=N/[C@@H](CCCNC(=N)N)C(=O)O)=C\c1cccs1 ; CCC(/C=N/[C@@H](CCCNC(=N)N)C(=O)O)=C\c1cccs1] 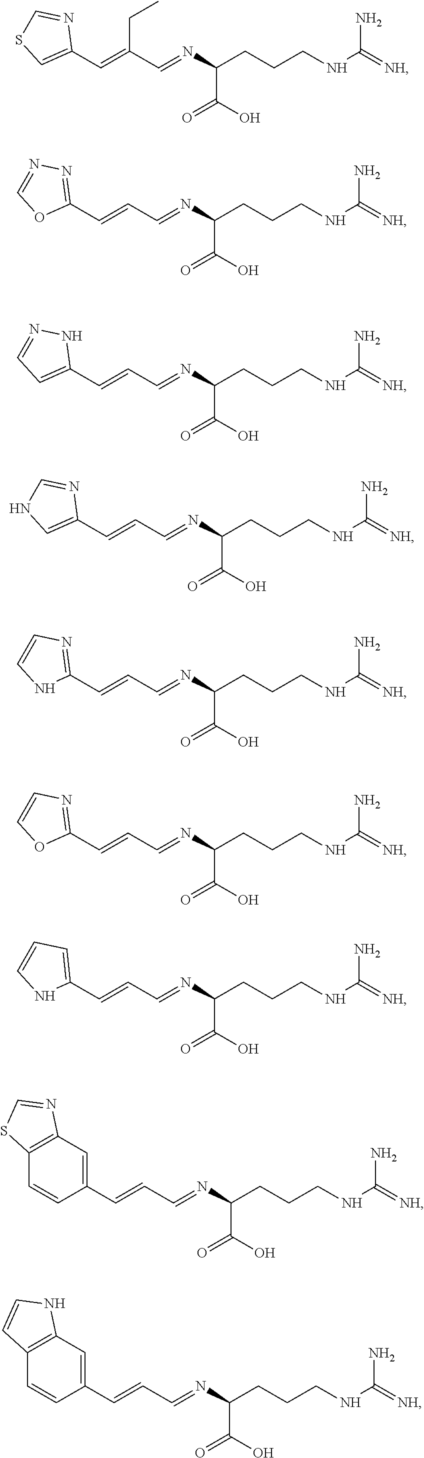 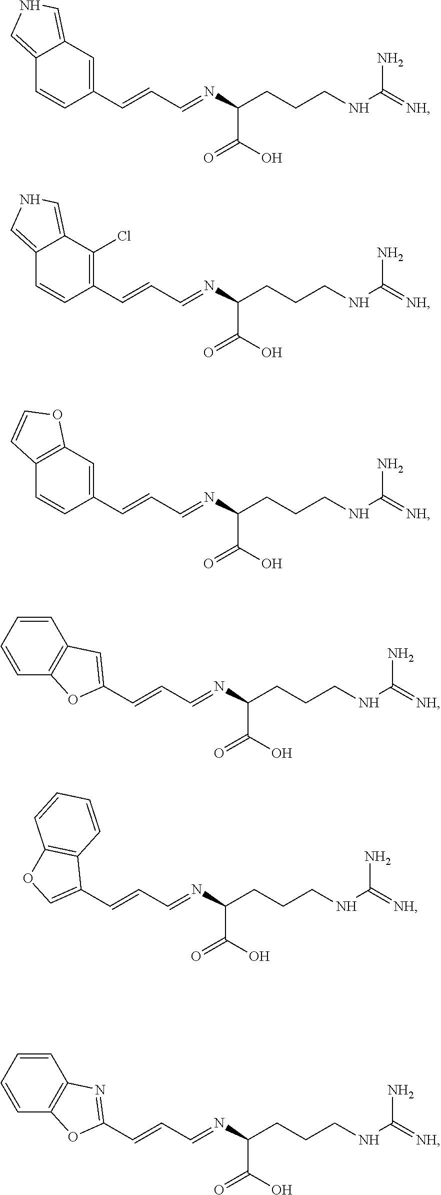 [SMILES: N=C(N)NCCC[C@H](/N=C/C=C/c1ccc2c[nH]cc2c1)C(=O)O ; N=C(N)NCCC[C@H](/N=C/C=C/c1ccc2c[nH]cc2c1Cl)C(=O)O ; N=C(N)NCCC[C@H](/N=C/C=C/c1ccc2ccoc2c1)C(=O)O ; N=C(N)NCCC[C@H](/N=C/C=C/c1cc2ccccc2o1)C(=O)O ; N=C(N)NCCC[C@H](/N=C/C=C/c1coc2ccccc12)C(=O)O ; N=C(N)NCCC[C@H](/N=C/C=C/c1nc2ccccc2o1)C(=O)O]
or agrochemically acceptable salts or N-oxides thereof.

39. The agricultural composition according to claim 36, wherein the compound is selected from 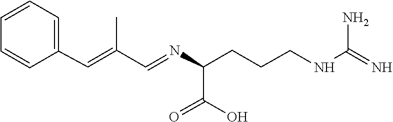 [SMILES: CC(/C=N/[C@@H](CCCNC(=N)N)C(=O)O)=C\c1ccccc1]
or agrochemically acceptable salts or N-oxides thereof.

40. The agricultural composition according to claim 36, wherein the compound is selected from 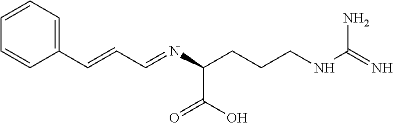 [SMILES: N=C(N)NCCC[C@H](/N=C/C=C/c1ccccc1)C(=O)O]
or agrochemically acceptable salts or N-oxides thereof.

41. The agricultural composition according to claim 36, wherein the composition is in the form of a solid, a liquid, a gel, or a paste.

42. The agricultural composition according to claim 41, wherein the solid composition is in the form of spheronised granules, extruded granules, water disintegrable granules, wettable powders, water dispersible granules, dustable powder, powder for dry seed treatment, water disintegrable tablet, or water dispersible powder for slurry seed treatment.

43. The agricultural composition according to claim 41, wherein the liquid composition is in the form of liquid suspension, suspension concentrate, suspoemulsion, oil dispersion, flowable concentrate, or a suspension concentrate for seed treatment.

44. A method of treating or controlling a parasitic nematode or a nematode infection in a plant comprising administering the compound of claim 27.

45. The method of claim 44, wherein the compound is selected from or agrochemically acceptable salts or N-oxides thereof. 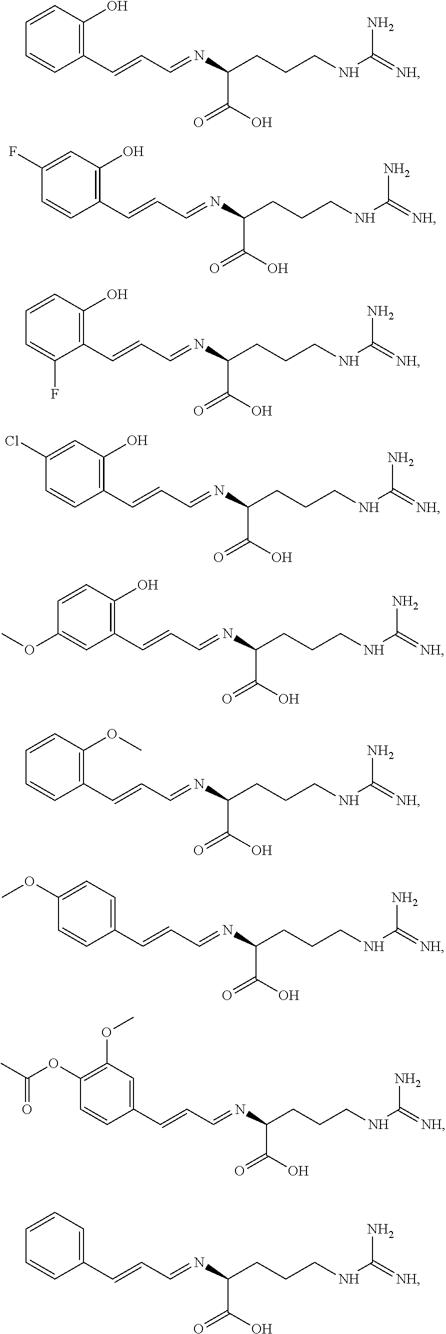 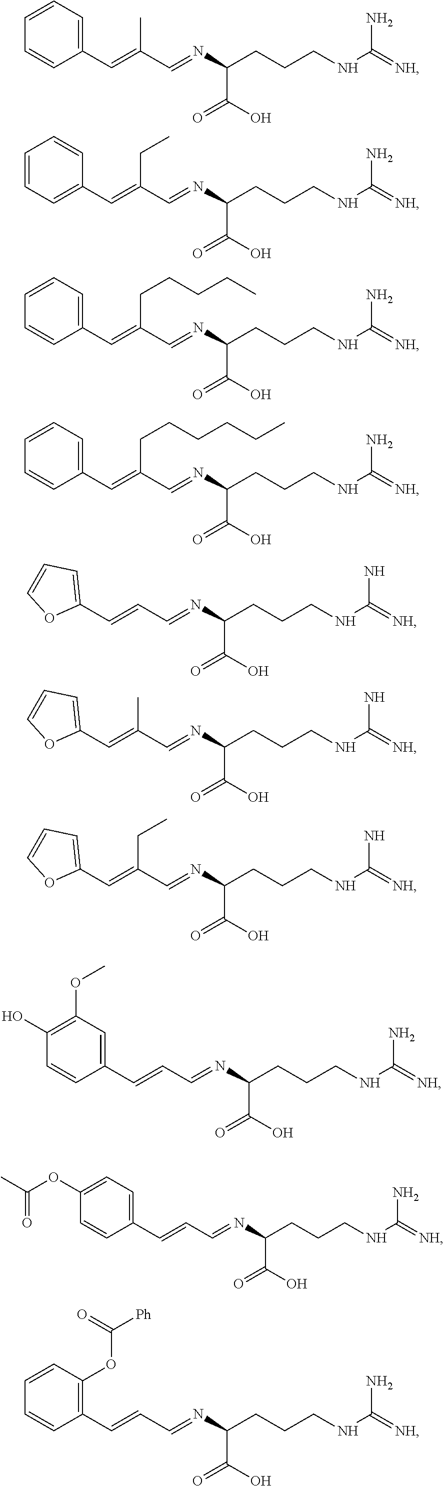 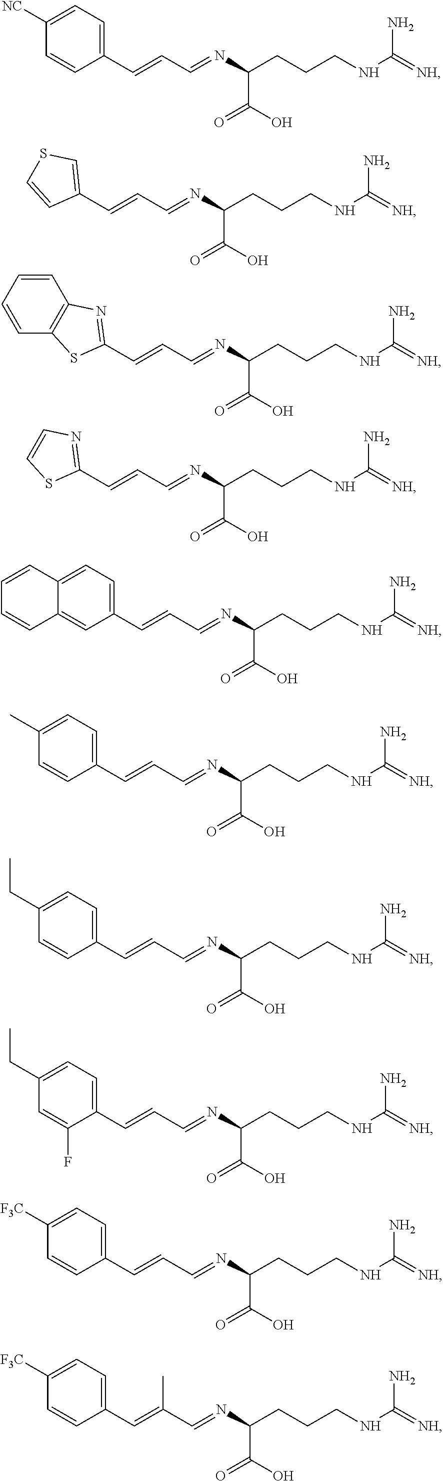 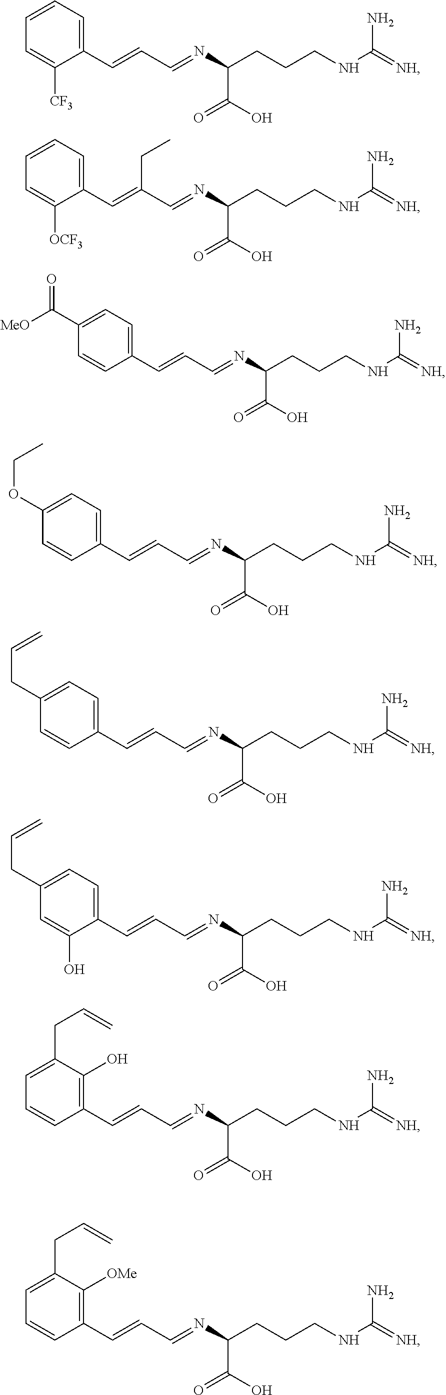 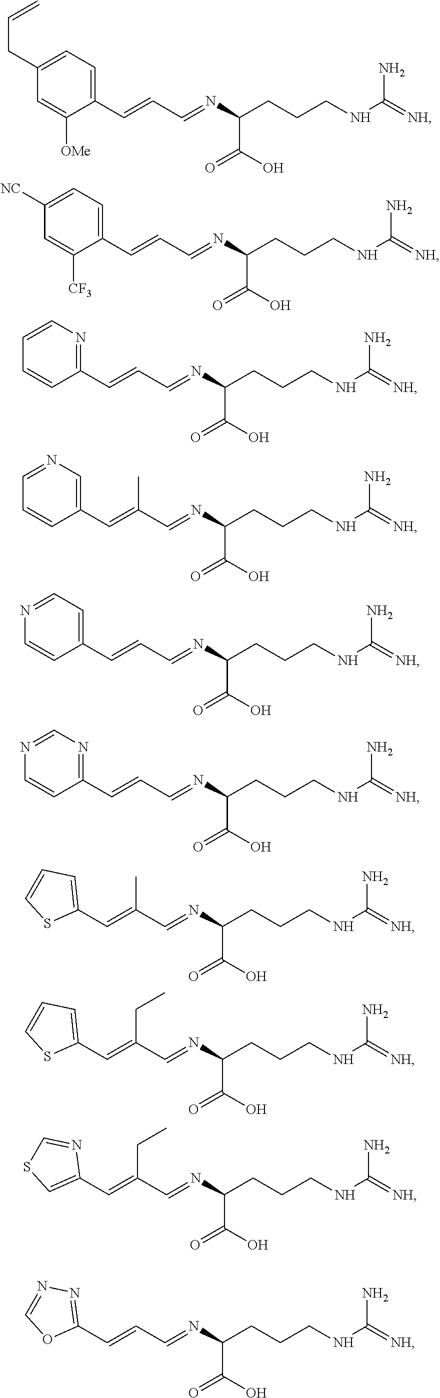 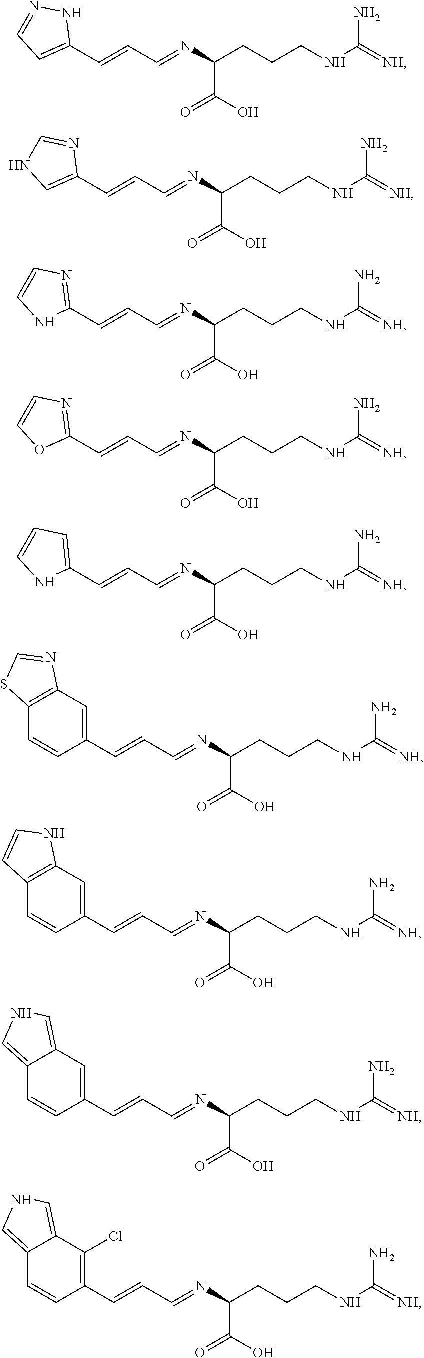 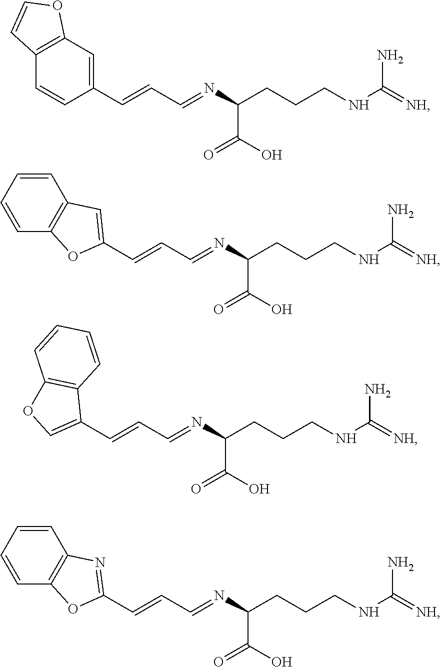 [SMILES: N=C(N)NCCC[C@H](/N=C/C=C/c1ccc2ccoc2c1)C(=O)O ; N=C(N)NCCC[C@H](/N=C/C=C/c1cc2ccccc2o1)C(=O)O ; N=C(N)NCCC[C@H](/N=C/C=C/c1coc2ccccc12)C(=O)O ; N=C(N)NCCC[C@H](/N=C/C=C/c1nc2ccccc2o1)C(=O)O]
or agrochemically acceptable salts or N-oxides thereof.

46. A process for the preparation of the compound of claim 27, comprising: combining a compound of formula (A) with a compound of Formula (B) [chemical structure]
